(19) United States
(12) Patent Application Publication
(10) Pub. No.: US 2026/0209855 A1
(43) Pub. Date: Jul. 23, 2026

(54) COMPOSITIONS AND METHODS FOR DETECTION AND TREATMENT OF PROSTATE CANCER

(71) Applicant: The Regents of the University of California, Oakland, CA (US)

(72) Inventor: Ida Deichaite, Del Mar, CA (US)

(21) Appl. No.: 19/118,665

(22) PCT Filed: Oct. 7, 2023
(86) PCT No.: PCT/US2023/076327

§ 371 Date: Apr. 4, 2025

Related U.S. Application Data
(60) Provisional application No. 63/378,802, filed on Oct. 7, 2022.
(60) Provisional application No. 63/470,010, filed on May 31, 2023.

(51) Int. Cl. C12Q 1/6886

(52) U.S. Cl. CPC C12Q 1/6886; C12Q 2600/118; C12Q 2600/158; C12Q 2600/16

(57) ABSTRACT

Compositions and methods are described herein that are useful for the detection of prostate cancer progression by determining the expression levels of genes associated with metastatic PCa. Based on the expression of IL-6, SELE, FOSB, NRK, NFRB2, FOXP3, ARG1, CEBPDP, TNFα, ADAMTS4, PENK, FOSL1, DUSP1, ACTA1, AGT, ATF3, CDK1, CXCL8, SELP, VCAN, TFP12, or NR4A3 genes or any combination thereof, treatment of PCa with SELE agonists, TNFα antagonists, and/or immune checkpoint inhibitors (ICIs) are shown to be effective.

### CROSS-REFERENCE TO RELATED APPLICATION

This application claims the priority of U.S. provisional application Ser. No. 63/378,802, filed Oct. 7, 2022, and U.S. provisional application Ser. No. 63/470,010, filed May 31, 2023, the disclosure of which is incorporated herein by reference in their entirety as if fully set forth herein.

### INCORPORATION BY REFERENCE OF SEQUENCE LISTING

This application contains a sequence listing. It has been submitted electronically as an XML file titled “1133.112WO1 Seq_List.xml.” The sequence listing is 988,585 bytes in size and was created on Oct. 7, 2023. It is hereby incorporated by reference in its entirety.

### BACKGROUND

Prostate cancer (PCa) is the most common solid organ cancer in men in the United States, with 191,930 new cases and 33,330 deaths in 2020. PCa ranks second in incidence and fifth in mortality among all malignancies. The life risk of PCa diagnosis is reported as one in nine men, but the risk of death may be as low as 2%. PCa is a heterogeneous disease, ranging from very slowly developing and slightly benign to progressing, aggressive, metastatic and fatal, even when properly treated.

The current recommendations for PCa diagnosis include analyzing the concentration of prostate-specific antigen (PSA), as well as conducting a digital rectal examination (DRE) for abnormalities. However, DRE has low sensitivity, while PSA is rather organ-, but not tumor-specific (low specificity), and has a low positive predictive value (~30%). The final diagnosis of PCa depends on the histopathological report of adenocarcinoma in the core biopsy of the prostate gland. False positive PSA test results, in patients with benign prostatic hyperplasia (BPH) and/or prostatitis, may result in systematic transrectal ultrasonography (TRUS)—controlled prostate biopsy (Bx). Additionally, PSA—based screening may lead to over-diagnosis and potentially over-treatment of PCa. There is a clinically unmet need to develop biomarkers that will help control PCa diagnosis and treatment strategies.

### SUMMARY

Compositions and methods are described herein that are useful for the detection of prostate cancer (PCa) disease progression by determining the expression levels of genes for IL-6, SELE, FOSB, NRK, NFKB2, FOXP3, ARG1, CEBPDP, TNFα, ADAMTS4, PENK, FOSL1, DUISP1, ACTA1, AGT, ATF3, CDK1, CXCL8, SELP, VCAN, TFPI2, NR4A3, or a combination thereof. The expression level of these genes is associated with risk of developing metastatic PCa. For example, surprisingly, opposing patterns of IL-6 and INFα expression were observed between localized and metastatic disease. IL-6 was robustly expressed in localized disease and downregulated in metastatic disease. The reverse was observed with TNFα expression. The results described herein indicate that gene expression of IL-6, SELE, FOSB, NRK, NFKB2 FOXP3, ARG1, CEBPDP, TNFα, ADAMTS4, PENK, FOSL1, DUSP1, ACTA1, AGT, ATF3, CDK1, CXCL8, SELP, VCAN, TFPI2, NR4A3, or a combination thereof, are prognostics tests for prostate cancer, useful to identify patients with a high risk of a poor outcome, hereby allowing those patients to be treated with additional cycles or combinations of therapies.

In embodiments, a decrease in expression of SELE, FOSB, NRK, NR4A3, and ADAMTS4 genes in prostate tumor tissue as compared to normal prostate tissue can be indicative of the prostate tumor having a high risk of becoming metastatic, even when the prostate tumor is in the early localized stage of PCa. The patient having a prostate tumor exhibiting such a decrease in expression of SELE, FOSB, NRK, NR4A3, and ADAMTS4 genes can be treated with a variety of TNFα antagonists and/or SELE agonists to boost SELE expression.

In addition, these signatures can be used as a predictive signature to select patients for treatments with anti-TNFα agents that modulate molecular targets to convert prostate cancer from a “cold cancer” that is unresponsive to immune checkpoint inhibitors (ICI) treatment to a “hot” one that becomes responsive to ICI treatment.

Described herein are methods that can include: (a) assaying a biological sample comprising prostate tissue from a subject for expression of genes comprising IL-6, SELE, FOSB, NRK, NFKB2, FOXP3, ARG1, CEBPDP, TNFα, ADAMTS4, PENK, FOSL1, DUSP1, ACTA1, AGT, ATF3, CDK1, CXCL8, SELP, VCAN, TFPI2, NR4A3, or combinations thereof, to determine one or more expression levels for the IL-6, SELE, FOSB, NRK, NFKB2, FOXP3, ARG1, CEBPDP, TNFα, ADAMTS4, PENK, FOSL1, DUSP1, ACTA1, ACT, ATF3, CDK1, CXCL8, SELP, VCAN, TFPI2, or NR4A3 genes; (b) comparing the determined expression levels with one or more reference values to identify any altered expression levels in the subject's biological sample, wherein altered expression levels of the IL-6, SELE, FOSB, NRK, NFKB2, FOXP3, ARG1, CEBPDP, TNFα, ADAMTS4, PENK, FOSL1, DUSP1, ACTA1, AGT, ATF3, CDK1, CXCL8, SELP, VCAN, TFPI2, NR4A3, or combinations thereof in the biological sample relative to the reference value indicates that the subject has PCa with a high risk of developing metastasized PCa; and (c) administering one or more TNFα antagonists and/or SELE agonists to a subject determined to have the high risk of developing metastasized PCa.

In embodiments, the amount of (level of expression of) RNA encoding a polypeptide having an amino acid sequence of SEQ ID NO: 1, 3, 5, 7, 9, 11, 13, 15, or 17, or a polypeptide having at least 80%, 82%, 85%, 87%, 88%, 89%, 90%, 92%, 94%, 95%, 97%, 98% or 99% amino acid sequence identity thereto, or a portion thereof, in a sample is determined. In embodiments, the amount of RNA encoding a polypeptide having at least two, three, four, or five of SEQ ID NO: 1, 3, 5, 7, 9, 11, 13, 15, or 17 or a polypeptide having at least 80%, 82%, 85%, 87%, 88%, 89%, 90%, 92%, 94%, 95%, 97%, 98% or 99% amino acid sequence identity thereto, or a portion thereof, is determined. In embodiments, the amount of RNA encoding a polypeptide having an amino acid sequence of SEQ ID NO: 3, 5, 7, 13, and 17 or a polypeptide having at least 80%, 82%, 85%, 87%, 88%, 89%, 90%, 92%, 94%, 95%, 97%, 98% or 99% amino acid sequence identity thereto, or a portion thereof, is determined.

Interleukin 6 (IL-6) in prostate cancer (PCa) is recognized as a potential mediator and biomarker of disease progression, Elevated IL-6 plasma levels have been implicated in PCa development and progression (Shariat et al., 2001; Smith et al., 2001). Local production of IL-6 has been detected in androgen-independent PCa cell lines, arguing for its involvement in autocrine and paracrine functions (Deeble et al., 2001; Twillie et al., 1995).

Both IL-6 and tumor necrosis factor alpha (TNFα) serum levels were shown to correlate with patient disease progression and survival, further establishing both cytokines as mediators and prognostic biomarkers (Michalaki et al., 2004). However, the role of IL-6 in disease progression remains contested. An IL-6 antagonist (siltuximab) has been tested in clinical trials in PCa patients but had no clinical efficacy (Fizazi et al., 2012). Other studies reported that in PCa patients, IL-6 is not detected in PCa cells apart from the stromal compartments (Yu et al., 2015). It remains to be firmly established whether IL-6 is a driver or a surrogate biomarker of PCa progression.

Prostate cancer (PCa) is a “cold” cancer which means that it is not very responsive to immune checkpoint inhibitors (ICI), such as PD-1, PD-L1, CTLA4, etc. The role of the inflammatory milieu in prostate cancer progression is not well understood. Differences in inflammatory signaling between localized and metastatic disease may point to opportunities for early intervention. PCa disease progression was modeled by analyzing RNA-seq of localized vs. metastatic patient samples, followed by CIBERSORTx to assess their immune cell populations. The VHA CDW registry of PCa patients was analyzed for anti-TNFα clinical outcomes. Statistically significant opposing patterns of IL-6 and TNFα expression were observed between localized and metastatic disease. IL-6 was robustly expressed in localized disease and downregulated in metastatic disease. The reverse was observed with TNFα expression. Metastatic disease was also characterized by downregulation of adhesion molecule E-Selectin, matrix metalloproteinase ADAMTS-4 and a shift to M2 macrophages whereas localized disease demonstrated a preponderance of M1 macrophages. Treatment with anti-TNFα agents was associated with earlier stage disease at diagnosis.

As disclosed herein, TNFα's suppressive function has to do with the lack of response to ICIs and several targets impacted by TN-Fa were identified that can be modulated to convert prostate cancer from a cold cancer to a hot one; and therefore, be responsive to ICI.

The genes associated with PCa disease progression prediction (prognostic value) are one or more, e.g., two, three, four, five, six, seven, eight, nine or more of: IL-6, SELE, FOSB, NRK, NFKB2, FOXP3, ARG1, CEBPDP, TNFα, ADAMTS4, PENK, FOSL1, DUSP1, ACTA1, AGT, ATF3, CDK1, CXCL8, SELP, VCAN, TFPI2, NR4A3, as well as NR4A1, NR4A2, and NR4A3.

The data points to clearly different inflammatory contexts between localized and metastatic prostate cancer. Primary localized disease demonstrates local inflammation and adaptive immunity, whereas metastases are characterized by immune cold microenvironments and a shift towards resolution of inflammation and tissue repair. Therapies that interfere with these inflammatory networks may offer opportunities for early intervention in monotherapy or in combination with immunotherapies and anti-angiogenic approaches, polarization, and/or immune remodeling.

In one embodiment, a method is provided to predict disease progression or a risk of disease progression in a mammal with prostate cancer, comprising: detecting in a physiological sample of the mammal expression of one or more of IL-6, SELE, FOSB, NRK, NFKB2, FOXP3, ARG1, CEBPDP, TNFα, ADAMTS4, PENK, FOSL1, DUSP1, ACTA1, AGT, ATF3, CDK1, CXCL8, SELP, VCAN, TFPI2, NR4A3, or any combination thereof; and determining whether the profile of expression is indicative of progression of prostate cancer. In one embodiment, the mammal is a human. In one embodiment, the sample is a tissue sample. In one embodiment, the sample is a physiological fluid sample having cells, e.g., a blood sample. In one embodiment, three or more of IL-6, SELE, FOSB, NRK, NFKB2, FOXP3, IL-6, ARG1, CEBPDP, TNFα, ADAMTS4, PENK, FOSL1, DUSP1, ACTA1, AGT, ATF3, CDK1, CXCL8, SELP, VCAN, and TFPI2 are detected. In one embodiment, five or more of IL-6, SELE, FOSB, NRK, NFKB2, FOXP3, IL-6, ARG1, CEBPDP, TNFα, ADAMTS4, PENK, FOSL1, DUSP1, ACTA1, AGT, ATF3, CDK1, CXCL8, SELP, VCAN, and TFPI2 are detected. In one embodiment, ten or more of IL-6, SELE, FOSB, NRK, NFKB2, FOXP3, IL-6, ARG1, CEBPDP, TNFα, ADAMTS4, PENK, FOSL1, DUSP1, ACTA1, AGT, ATF3, CDK1, CXCL8, SELP, VCAN, and TFPI2 are detected. In one embodiment, IL-6, SELE, FOSB, NRK, NFKB2, FOXP3, IL-6, ARG1, CEBPDP, TNFα, ADAMTS4, PENK, FOSL1, DUSP1, ACTA1, AGT, ATF3, CDK1, and CXCL8, SELP, VCAN, and TFPI2 are detected. In one embodiment, RNA expression is detected. In one embodiment, protein products of the genes are detected.

Further provided is a method of inhibiting or treating disease progression in a mammal with prostate cancer, comprising: administering to the mammal an effective amount of a TNFα inhibitor or an anti-angiogenic agent, or both, wherein the mammal has an expression profile of one or more of IL-6, SELE, FOSB, NRK, NFKB2, FOXP3, IL-6, ARG1, CEBPDP, TNFα, ADAMTS4, PENK, FOSL1, DUSP1, ACTA1, AGT, ATF3, CDK1, CXC18, or any combination thereof, that is indicative of increased risk of disease progression. In one embodiment, the mammal is a human. In one embodiment, the sample is a tissue sample. In one embodiment, three or more of IL-6, SELE, FOSB, NRK, NFKB2, FOXP3, IL-6, ARG1, CEBPDP, TNFα, ADAMTS4, PENK, FOSL1, DUSP1, ACTA1, AGT, ATF3, CDK1, CXCL8 are detected. In one embodiment, RNA expression is detected. In one embodiment, an immune checkpoint inhibitor is administered.

### BRIEF DESCRIPTION OF THE FIGURES

FIGS. 1A-1H show somatic tumor RNA-sequence data for expression levels of TNFα and IL-6 in localized versus metastatic PCa. FIG. 1A is a graph of Log₂TPM (“TPM” is transcripts per million) expression of IL-6, TNFα, FOSB, and CEBPD in the PCa cohort split by localized and metastatic status. These genes were measured to have significantly differential expression between the localized and metastatic groups (two-tailed student's t-test). Median and interquartile ranges are shown for each group. FIG. 1B-1E are heatmaps showing relative expression profiles (mRNA) of selected genes vs. localized/metastatic groupings of 49 PCa patients, where 28 were localized (black) and 21 were metastatic (grey). The genes shown are TNFα and IL-6 (FIG. 1B), AP-1 components (FIG. 1C), NF-kB (FIG. 1D), and CEBP family genes (FIG. 1E). On the right side of the heatmaps, the Information Coefficient (Δ) is shown, which is a measure of association of each gene/gene set vs. the phenotype and the corresponding permutation-derived p-value. FIG. 1F is a table shown mean values and standard deviations for all components of the AP-1 complex. FIGS. 1G and 1H are heatmaps showing single-sample GSEA analysis of the Information Coefficient (Δ) association between localized and metastatic groups and the expression of NF-kB related gene sets (FIG. 1G) and AP-1 related gene sets (FIG. 1H). The 5 top scoring gene set are shown.

FIGS. 2A-2G are scatterplots with Pearson's R calculated between AP-1 Complex subunit genes and TNFα/IL-6 log 2TPM expression values from RNA-seq analysis of 49 PCa patients. “r”: Pearson's R; “p”: P-Value; “Localized”: N=28 localized patients; “Metastatic”: N=21 metastatic patients; “TPM”: transcripts per million. FIG. 2A shows significant linear correlation between IL-6 and FOSB in the metastatic state (metastatic: r=0.79 and p=2e-05; localized: r=0.45 and p=0.01612). FIG. 2B shows significant linear correlation between IL-6 and FOS in the metastatic state (metastatic: r=0.73 and p=0.00017; localized: r=0.325 and p=0.09131). FIG. 2C shows significant linear correlation between IL-6 and JUN in the metastatic state (metastatic: r=0.64 and p=0.00177; localized: r=0.328 and p=0.08883). FIG. 2D shows that the correlation between TNFα and FOSB is not significant in the metastatic state and has a weak linear correlation in localized disease (metastatic: r=0.072 and p=0.75719; localized: r=0.367 and p=0.05486). FIG. 2E shows that the correlation between TNFα and FOS is not significant in the metastatic state and has a weak linear correlation in localized disease (metastatic: r=0.113 and p=0.6269; localized: r=0.458 and p=0.01432). FIG. 2F shows that the correlation between TNFα and JUN is not significant in the metastatic state and has a weak linear correlation in localized disease (metastatic: r=0.043 and p=0.85233; localized: r=0.489 and p=0.00825). FIG. 2G that TNFα and IL-6 are neither correlated significantly with each other in localized nor in metastatic groups (metastatic: r=0.195 and p=0.39808; localized: r=0.157 and p=0.42377).

FIG. 3 shows pie-charts representing the percentage of cells where the expression of the TNFα, JUN, FOS, IL6, JUND, FOSB, and SELE genes was detected. The size of each circle in the “Total Cells” column qualitatively represents the proportion of that cell type within the total data.

FIGS. 4A-4E show single-sample Gene Set Enrichment Analysis (GSEA) analysis heatmaps showing the association between IL-6 and TNFα and the expression of and AP-1 and NF-kB related gene sets. FIG. 4A is a heatmap of the top scoring genes representing the AP-1 transcription factor network. FIG. 4B is a heatmap of the top scoring genes regulated by NF-κB. FIG. 4C is a heatmap of the top scoring genes sets for TNFα (mRNA) that produced genes representing cancer motility and invasion genes up-regulated by the AP-transcription factor. FIG. 4D is a heatmap of the top scoring genes sets for TNFα (mRNA) that produced genes representing genes TNF receptor superfamily (TNFSF) members mediating the non-canonical NF-κB pathway. FIG. 4E is a heatmap showing positive correlation between the relative expression (mRNA) of selected genes, versus the profile of IL-6. The information coefficient (A) and associated p-values are shown on the right side of the heatmap. The top 10 scoring genes are shown.

FIGS. 5A-5D are an analysis of SELE gene expression in a patient cohort. FIG. 5A is a heatmap showing relative expression profiles (mRNA) of TNFα, IL-6, and SELE vs localized/metastatic groupings of 49 PCa patients, where 28 were localized (black) and 21 were metastatic (grey). FIG. 5B is a graph showing Log₂TPM expression of SELE cohort split by localized and metastatic status. Median and interquartile ranges are shown. FIGS. 5C and 5D are single-sample GSEA analysis heatmaps showing NF-κB related gene sets (FIG. 5C) and AP-1 related gene sets (FIG. 5D) vs. the relative expression (mRNA) of SELE.

FIGS. 6A-6E are Scatterplots with Pearson's R calculated between SELE, IL-6, TNFα, and AP-1 log₂TPM expression values from RNA-seq analysis of 49 PCa patients. “r”: Pearson's R; “p”: P-Value; “Localized”: N=28 localized patients; “Metastatic”: N=21 metastatic patients; “TPM”: transcripts per million. FIG. 6A shows significant linear correlation between SELE and IL-6 in both localized and the metastatic state, with a stronger correlation in the localized state (metastatic: r=0.511 and p=0.0178; localized: r=0.798 and p=3.6e-07). FIG. 6B shows significant linear correlation between SELE and FOSB in both localized and the metastatic state (metastatic: r=0.595 and p=0.0044; localized: r=0.619 and p=0.0004). FIG. 6C shows significant linear correlation between SELE and JUN in both localized and the metastatic state (metastatic: r=0.579 and p=0.0059; localized: r=0.517 and p=0.0048). FIG. 6D shows significant linear correlation between SELE and JUN in both localized and the metastatic state (metastatic: r=0.609 and p=0.0034; localized: r=0.597 and p=0.0008). FIG. 6E shows low correlation between SELE and TNFα (metastatic: r=0.105 and p=6504; localized: r=0.323 and p=0.0931).

FIGS. 7A-7F show M1 and M2 macrophage enrichment in metastatic disease. FIG. 7A shows M1 macrophage infiltration of metastatic vs. pre-metastatic patients in batch 1. FIG. 7B shows M1 macrophage infiltration of metastatic vs. pre-metastatic patients in batch 2. FIG. 7C shows M1 macrophage infiltration of metastatic vs. pre-metastatic patients in batch 3. FIG. 7D shows M2 macrophage infiltration of metastatic vs. pre-metastatic patients in batch 1. FIG. 7E shows M2 macrophage infiltration of metastatic vs. pre-metastatic patients in batch 2. FIG. 7F shows M2 macrophage infiltration of metastatic vs. pre-metastatic patients in batch 3. Infiltration estimates were generated using a gene signature matrix derived from an annotated single cell RNA-seq dataset of localized and metastatic PCa samples.

FIGS. 8A-8C show M1 to M2 macrophage infiltration ratio estimates across each cohort, split by localized vs metastatic status. M1 and M2 infiltration estimates were subjected to a pseudocount to avoid infinite ratios. FIG. 8A shows M1 to M2 macrophage infiltration ratio estimates in the batch 1 study cohort (n=120) that consisted of 79 localized and 49 metastatic PCa samples. FIG. 8B shows M1 to M2 macrophage infiltration ratio estimates in the batch 2 study cohort (n=41) that consisted of 23 localized and 18 metastatic PCa samples. FIG. 8C shows M1 to M2 macrophage infiltration ratio estimates in the batch 3 cohort (n=49) that consisted of 28 localized and 21 metastatic PCa samples.

FIG. 9 is a graph of Log₂TPM expression of ARG1, FOXS1, and ADAMTS-4 in PCa cohort split by localized and metastatic status. These genes were measured to have significantly differential expression between the localized and metastatic groups (two-tailed student's t-test). Median and interquartile ranges are shown below for each group.

FIGS. 10A-10J are graphs showing gene expression in TPM for the indicated gene for tumors biopsied at a primary (localized) and metastatic site, asterisk indicates significance p<0.05). FIG. 10A shows expression of the NRK gene in a primary (localized) and metastatic site. FIG. 10B shows expression of the SELE gene in a primary (localized) and metastatic site. FIG. 10C shows expression of the NF-κB2 gene in a primary (localized) and metastatic site. FIG. 10D shows expression of the FOXP3 gene in a primary (localized) and metastatic site. FIG. 10E shows expression of the IL6 gene in a primary (localized) and metastatic site. FIG. 10F shows expression of the CEBPDP gene in a primary (localized) and metastatic site. FIG. 10G shows expression of the TNFα gene in a primary (localized) and metastatic site. FIG. 10H shows expression of the FOSB gene in a primary (localized) and metastatic site. FIG. 10I shows expression of the IL10 gene in a primary (localized) and metastatic site. FIG. 10J shows expression of the ADAMTS4 gene in a primary (localized) and metastatic site.

FIGS. 11A-11B show the correlation between FOSB and upstream genes for tumors biopsied at either a primary or metastatic site. FIG. 11A are scatterplots FOSB expression vs TNFα, SELE, and IL6 genes and the coefficient of determination (Pearson's) for tumors biopsied at either a primary or metastatic site. FIG. 11B is a heatmap of Pearson's correlation coefficients for the TNFα, SELE, and IL6 genes.

FIGS. 12A-12B show the correlation between TNFα and immune checkpoint genes for tumors biopsied at either a primary or metastatic site. A strong relationship is observed between immune check point genes and the TNFα ligand. FIG. 12A is a scatterplot for TNFα vs CTLA4, PDCD1, and CD247 genes and the coefficient of determination (Pearson's) for tumors biopsied at either a primary or metastatic site. FIG. 12B Heatmap of Pearson's correlation coefficients for the CTLA4, PDCD1, and CD247 genes.

FIGS. 13A-13C show clinical outcomes data of gene expression levels in primary vs metastatic sites. FIG. 13A is a table of Hazard Ratios (HR) ratios segmented on gene expression (Q1 vs Q4) for tumor biopsies taken from the primary or metastatic site. FIG. 13B is a Kaplan-Meier curve for high and low expression of ADAMTS4 in the primary site. FIG. 13C is a Kaplan-Meier curve for high and low expression of ADAMTS4 in the metastatic site.

FIG. 14 is a graph showing TNFα expression vs. overall survival (OS) in days from start of treatment with immune checkpoint inhibitors (ICI) for Q1 vs Q4 in primary prostate cancer.

FIGS. 15A and 15B are graphs showing CEBPD expression vs overall survival (OS) in days from start of treatment with immune checkpoint inhibitors (ICI) for Q1 vs Q4 in primary (FIG. 15A) and metastatic (FIG. 15B) prostate cancer.

FIG. 16 is a graph showing SELE expression vs. overall survival (OS) in days from start of treatment with immune checkpoint inhibitors (ICI) for Q1 vs Q4 in primary prostate cancer.

FIGS. 17A and 17B. are graphs showing ADAMTS4 expression vs overall survival (OS) in days from start of treatment with immune checkpoint inhibitors (ICI) for Q1 vs Q4 in primary (FIG. 17A) and metastatic (FIG. 17B) prostate cancer.

FIG. 18 is a table showing clinical outcome data vs gene expression levels in primary vs metastatic sites with ICI treatment. Hazard Ratios (HR) ratios are segmented on gene expression (Q1 vs Q4) for tumor biopsies taken from the primary or metastatic site.

FIG. 19 is a scatter plot showing the correlation between FOSB and TNFα, SELE and IL6 expression.

FIG. 20 is a graph showing a normalized enrichment score comparing primary vs metastatic prostate tumors. Positive Normalized enrichment score (NES) suggests enrichment in the primary cohort whereas a negative NES is associated with enrichment in the metastatic cohort. Normalized enrichment score comparing Primary vs Metastatic prostate tumors. all gene sets shown were significantly enriched (p<0.05, FDR<0.1.

FIGS. 21A-21D are graphs showing overall survival (OS) in days for Q1 vs Q4 expression quartiles of different immune related genes in primary and metastatic prostate cancer. FIG. 21A shows OS vs. TNFα expression in primary PCa. FIG. 21B shows OS vs. TNFα expression in metastatic PCa. FIG. 21C shows OS vs. IL-6 expression in primary PCa. FIG. 21D shows OS vs. IL-6 expression in metastatic PCa.

FIGS. 22A-22D are graphs showing overall survival (OS) in days for Q1 vs Q4 expression quartiles of different immune related genes in primary and metastatic prostate cancer. FIG. 22A shows OS vs. IL-10 expression in primary PCa. FIG. 22B shows OS vs. IL-10 expression in metastatic PCa. FIG. 22C shows OS vs. FOXP3 expression in primary PCa. FIG. 22D shows OS vs. FOXP3 expression in metastatic PCa.

FIGS. 23A-23D are graphs showing overall survival (OS) in days for Q1 vs Q4 expression quartiles of different immune related genes in primary and metastatic prostate cancer. FIG. 23A shows OS vs. CEPBD expression in primary PCa. FIG. 23B shows OS vs. CEPBD expression in metastatic PCa. FIG. 23C shows OS vs. NF-κB expression in primary PCa. FIG. 23D shows OS vs. NF-κB expression in metastatic PCa.

FIGS. 24A-24D are graphs showing overall survival (OS) in days for Q1 vs Q4 expression quartiles of different immune related genes in primary and metastatic prostate cancer. FIG. 24A shows OS vs. FOSB expression in primary PCa. FIG. 24B shows OS vs. FOSB expression in metastatic PCa. FIG. 24C shows OS vs. NRK expression in primary PCa. FIG. 24D shows OS vs. NRK expression in metastatic PCa.

FIGS. 25A-25D are graphs showing overall survival (OS) in days for Q1 vs Q4 expression quartiles of different immune related genes in primary and metastatic prostate cancer. FIG. 25A shows OS vs. SELE expression in primary PCa. FIG. 25B shows OS vs. SELE expression in metastatic PCa. FIG. 25C shows OS vs. ADAMTS4 expression in primary PCa. FIG. 25D shows OS vs. ADAMTS4 expression in metastatic PCa.

FIGS. 26A-26D are graphs showing overall survival (OS) in days from the start of ICI treatment for Q1 vs Q4 expression quartiles of different immune related genes in primary and metastatic prostate cancer. FIG. 26A shows OS in days from the start of ICI treatment vs. TNFα expression in primary PCa. FIG. 26B shows OS in days from the start of ICI treatment vs. TNFα expression in metastatic PCa. FIG. 26C shows OS in days from the start of ICI treatment vs. IL-6 expression in primary PCa. FIG. 26D shows OS in days from the start of ICI treatment vs. IL-6 expression in metastatic PCa.

FIGS. 27A-27D are graphs showing overall survival (OS) in days from the start of ICI treatment for Q1 vs Q4 expression quartiles of different immune related genes in primary and metastatic prostate cancer. FIG. 27A shows OS in days from the start of ICI treatment vs. IL-10 expression in primary PCa. FIG. 27B shows OS in days from the start of ICI treatment vs IL-10 expression in metastatic PCa. FIG. 27C shows OS in days from the start of ICI treatment vs. FOXP3 expression in primary PCa. FIG. 27D shows OS in days from the start of ICI treatment vs. FOXP3 expression in metastatic PCa.

FIGS. 28A-28D are graphs showing overall survival (OS) in days from the start of ICI treatment for Q1 vs Q4 expression quartiles of different immune related genes in primary and metastatic prostate cancer. FIG. 28A shows OS in days from the start of ICI treatment vs. CEBPD expression in primary PCa. FIG. 28B shows OS in days from the start of ICI treatment vs CEBPD expression in metastatic PCa. FIG. 28C shows OS in days from the start of ICI treatment vs. NF-κB expression in primary PCa. FIG. 28D shows OS in days from the start of ICI treatment vs. NF-κB expression in metastatic PCa.

FIGS. 29A-29D are graphs showing overall survival (OS) in days from the start of ICI treatment for Q1 vs Q4 expression quartiles of different immune related genes in primary and metastatic prostate cancer. FIG. 29A shows OS in days from the start of ICI treatment vs. FOSB expression in primary PCa. FIG. 29B shows OS in days from the start of ICI treatment vs FOSB expression in metastatic PCa. FIG. 29C shows OS in days from the start of ICI treatment vs. NRK expression in primary PCa. FIG. 29D shows OS in days from the start of ICI treatment vs. NRK expression in metastatic PCa.

FIGS. 30A-30D are graphs showing overall survival (OS) in days from the start of ICI treatment for Q1 vs Q4 expression quartiles of different immune related genes in primary and metastatic prostate cancer. FIG. 30A shows OS in days from the start of ICI treatment vs. SELE expression in primary PCa. FIG. 30B shows OS in days from the start of ICI treatment vs SELE expression in metastatic PCa. FIG. 30C shows OS in days from the start of ICI treatment vs. ADAMTS4 expression in primary PCa. FIG. 30D shows OS in days from the start of ICI treatment vs. ADAMTS4 expression in metastatic PCa.

FIG. 31 is a graph showing the difference in immune infiltrate between primary and metastatic tumors calculated using quantiseq (p<0.05).

FIGS. 32A-32C show SELE gene expression in primary versus metastatic tumors. FIG. 32A is a bar graph showing SELE gene expression in primary vs metastatic tumors. FIG. 32B shows scatterplots for FOSB vs TNFα and the coefficient of determination (Pearson's) for tumors biopsied at either a primary (upper plot) or metastatic (lower plot) site. FIG. 32C shows scatterplots for FOSB vs SELE and the coefficient of determination (Pearson's) for tumors biopsied at either a primary (upper plot) or metastatic (lower plot) site.

FIGS. 33A and 33B show the transcriptional profiles of TNFα, VCAM1, ICAM1, SELP, and SELE genes in prostate tumors (upper panel) and normal prostate tissue (lower panel) (FIG. 33A). FIG. 33B shows association matrices and a summary association matrix showing expression of SELP and SELE significantly reduces to undetectable levels in prostate tumors (PT) as compared to normal prostate (NP) tissue.

FIGS. 34A and 34B show hazard ratios of high vs low expressors of SELE, SELP, ICAM, and VCAM1. FIG. 34A are graphs showing overall survival vs expression of the indicated genes. FIG. 34B is a summary table of hazard ratio (HR), P value, confidence interval (CI), and median difference (in days).

FIGS. 35A and 35B show the transcriptional profiles of TNFα, VCAN, BCAN, and ADAMTS4 genes in prostate tumors (upper panel) and normal prostate tissue (lower panel) (FIG. 35A). FIG. 35B shows association matrices and a summary association matrix.

FIG. 36 is a table showing the correlation between Type 1 interferons and TNFα in PCa. Reduced interferon expression is found in the PCa vs normal prostate.

FIG. 37 shows Table 1 containing infiltration estimates in metastatic vs. pre-metastatic samples across all cohorts.

### DETAILED DESCRIPTION

To gain a better understanding of the role of proinflammatory cytokines in PCa progression, messenger RNA (mRNA) levels of IL-6 and TNFα from 49 somatic tumor tissue samples were analyzed. Somewhat contrary to published reports, it was found that IL-6 expression decreased with disease progression as compared to localized tumors. However, TNFα expression levels increased through disease progression. The IL-6 and TNFα expression data are in agreement with the results reported by Yu et al. (2015) who examined the cellular origin of IL-6 and TNFα in PCa patients utilizing quantitative reverse transcription PCR (q-RT-PCR) as well as chromogenic in situ hybridization (CISM) studies. They reported that benign prostate tissue had higher expression of IL-6 mRNA than matched patient tumor samples while TNFα expression remained unchanged.

While there is cumulative evidence that both IL-6 and TNFα play an important role in inflammation and PCa progression, the regulatory pathways and the immune microenvironment associated with these cytokines are not well understood and deciphering their function will aid in developing new therapeutic options for patients.

### Samples

PCa can be assessed through the evaluation of expression patterns, or profiles, of the IL-6, SELE, FOSB, NRK, NFKB2, FOXP3, ARG1, CEBPDP, TNFα, ADAMTS4, PENK, FOSL1, DUSP1, ACTA1, AGT, ATF3, CDK1, CXCL8, SELP, VCAN, TFPI2, NR4A3 genes in one or more subject samples. The term subject, or subject sample, refers to an individual regardless of health and/or disease status. A subject can be a subject, a study participant, a control subject, a screening subject, or any other class of individual from whom a sample is obtained and assessed using the markers and/or methods described herein. Accordingly, a subject can be diagnosed with prostate cancer, can present with one or more symptoms of prostate cancer, or a predisposing factor, such as a family (genetic) or medical history (medical) factor, for prostate cancer, can be undergoing treatment or therapy for prostate cancer, or the like. Alternatively, a subject can be healthy with respect to any of the aforementioned factors or criteria. It will be appreciated that the term “healthy” as used herein, is relative to prostate cancer status, as in the individual has normal prostate tissue, as the term “healthy” cannot be defined to correspond to any absolute evaluation or status. Thus, an individual defined as healthy with reference to any specified disease or disease criterion, can in fact be diagnosed with any other one or more diseases, or exhibit any other one or more disease criterion, including one or more cancers other than prostate cancer. However, the healthy controls are preferably free of any cancer.

In some cases, the methods for detecting, predicting, and/or assessing the prognosis of prostate cancer include collecting a biological sample comprising a cell or tissue, such as a prostate tissue sample or a primary prostate tumor tissue sample. By “biological sample” is intended any sampling of cells, tissues, or bodily fluids in which expression of IL-6, SELE, FOSB, NRK, NFKB2, FOXP3, ARG1, CEBPDP, TNFα, ADAMTS4, PENK, FOSL1, DUSP1, ACTA1, AGT, ATF3, CDK1, CXCL8, SELP, VCAN, TFPI2, and/or NR4A3 genes can be detected. Examples of such biological samples include, but are not limited to, biopsies and smears. Bodily fluids useful in the present invention include blood, lymph, urine, saliva, gynecological (e.g. seminal) fluids, or any other bodily secretion or derivative thereof. Blood can include whole blood, plasma, serum, or any derivative of blood. In some embodiments, the biological sample includes prostate cells, particularly prostate tissue from a biopsy, such as a prostate tumor tissue sample. Biological samples may be obtained from a subject by a variety of techniques including, for example, by scraping or swabbing an area, by using a needle to aspirate cells or bodily fluids, or by removing a tissue sample (i.e., biopsy). In some embodiments, a prostate tissue sample is obtained by, for example, fine needle aspiration biopsy, core needle biopsy, or excisional biopsy.

The samples can be stabilized for evaluating and/or quantifying IL-6, SELE, FOSB, NRK, NFKB2, FOXP3, ARG1, CEBPDP, TNFα, ADAMTS4, PENK, FOSL1, DUSP1, ACTA1, AGT, ATF3, CDK1, CXCL8, SELP, VCAN, TFPI2, and/or NR4A3 expression levels.

In some cases, fixative and staining solutions may be applied to some of the cells or tissues for preserving the specimen and for facilitating examination. Biological samples, particularly prostate tissue samples, may be transferred to a glass slide for viewing under magnification. In one embodiment, the biological sample is a formalin-fixed, paraffin-embedded prostate tissue sample, particularly a primary prostate tumor sample.

### Gene Expression

Various methods can be used for evaluating and/or quantifying IL-6, SELE, FOSB, NRK, NFKB2, FOXP3, ARG1, CEBPDP, TNFα, ADAMTS4, PENK, FOSL1, DUSP1, ACTA1, AGT, ATF3, CDK1, CXCL8, SELP, VCAN, TFPI2, or NR4A3 expression levels. By “evaluating and/or quantifying” is intended determining the quantity or presence of an RNA transcript or its expression product of IL-6, SELE, FOSB, NRK, NFKB2, FOXP3, ARG1, CEBPDP, TNFα, ADAMTS4, PENK, FOSL1, DUSP1, ACTA1, AGT, ATF3, CDK1, CXCL8, SELP, VCAN, TFPI2, or NR4A3 genes.

Methods for detecting expression of the IL-6, SELE, FOSB, NRK, NFKB2, FOXP3, ARG1, CEBPDP, TNFα, ADAMTS4, PENK, FOSL1, DUSP1, ACTA1, AGT, ATF3, CDK1, CXCL8, SELP, VCAN, TFPI2, or NR4A3 genes, including gene expression profiling, can involve methods based on hybridization analysis of polynucleotides, methods based on sequencing of polynucleotides, immunohistochemistry methods, and proteomics-based methods. The methods generally involve detect expression products (e.g., mRNA or proteins) encoding by the IL-6, SELE, FOSB, NRK, NFKB2, FOXP3, ARG1, CEBPDP, TNFα, ADAMTS4, PENK, FOSL1, DUSP1, ACTA1, AGT, ATF3, CDK1, CXCL8, SELP, VCAN, TFPI2, or NR4A3 genes. In some cases, PCR-based methods, which can include reverse transcription PCR (RT-PCR) (Weis et al., TIG 8:263-64, 1992), array-based methods such as microarray (Schena et al., Science 270:467-70, 1995), or combinations thereof are used. By “microarray” is intended an ordered arrangement of hybridizable array elements, such as, for example, polynucleotide probes, on a substrate. The term “probe” refers to any molecule that is capable of selectively binding to a specifically intended target biomolecule, for example, a nucleotide transcript or a protein encoded by or corresponding to IL-6, SELE, FOSB, NRK, NFKB2, FOXP3, ARG1, CEBPDP, TNFα, ADAMTS4, PENK, FOSL1, DUSP1, ACTA1, AGT, ATF3, CDK1, CXCL8, SELP, VCAN, TFPI2, or NR4A3 genes. Probes can be synthesized or obtained from IL-6, SELE, FOSB, NRK, NFKB2, FOXP3, ARG1, CEBPDP, TNFα, ADAMTS4, PENK, FOSL1, DUSP1, ACTA1, AGT, ATF3, CDK1, CXCL8, SELP, VCAN, TFPI2, or NR4A3 nucleic acids or they can be derived from appropriate biological preparations. Probes may be specifically designed to be labeled. Examples of molecules that can be utilized as probes include, but are not limited to, RNA, DNA, proteins, antibodies, and organic molecules.

Many expression detection methods use isolated RNA. The starting material is typically total RNA isolated from a biological sample, such as a cell or tissue sample, a tumor or tumor cell line, a corresponding normal tissue or cell line, or a combination thereof. If the source of RNA is a sample from a subject, RNA (e.g., mRNA) can be extracted, for example, from stabilized, frozen or archived paraffin-embedded, or fixed (e.g., formalin-fixed) tissue samples (e.g., pathologist-guided tissue core samples).

General methods for RNA extraction are available and are disclosed in standard textbooks of molecular biology, including Ausubel et al., ed., Current Protocols in Molecular Biology, John Wiley & Sons, New York 1987-1999. Methods for RNA extraction from paraffin embedded tissues are disclosed, for example, in Rupp and Locker (Lab Invest. 56:A67, 1987) and De Andres et al. (Biotechniques 18:42-44, 1995). In some cases, RNA isolation can be performed using a purification kit, a buffer set and protease from commercial manufacturers, such as Qiagen (Valencia, Calif.), according to the manufacturer's instructions. For example, total RNA from cells can be isolated using Qiagen RNeasy mini-columns. Other commercially available RNA isolation kits include MASTERPURE™ Complete DNA and RNA Purification Kit (Epicentre, Madison, Wis.) and Paraffin Block RNA Isolation Kit (Ambion, Austin, Tex.). Total RNA from tissue samples can be isolated, for example, using RNA Stat-60 (Tel-Test, Friendswood, Tex.). RNA prepared from tissue or cell samples (e.g. tumors) can be isolated, for example, by cesium chloride density gradient centrifugation. Additionally, large numbers of tissue samples can readily be processed using available techniques, such as, for example, the single-step RNA isolation process of Chomczynski (U.S. Pat. No. 4,843,155).

Isolated RNA can be used in hybridization or amplification assays that include, but are not limited to, PCR analyses and probe arrays. One method for the detection of RNA levels involves contacting the isolated RNA with a nucleic acid molecule (probe) that can hybridize to the mRNA encoded by the gene being detected. The nucleic acid probe can be, for example, a full-length cDNA, or a portion thereof, such as an oligonucleotide of at least 7, 15, 30, 60, 100, 250, or 500 nucleotides in length and sufficient to specifically hybridize under stringent conditions to any of the IL-6, SELE, FOSB, NRK, NFKB2, FOXP3, ARG1, CEBPDP, TNFα, ADAMTS4, PENK, FOSL1, DUSP1, ACTA1, AGT, ATF3, CDK1, CXCL8, SELP, VCAN, TFPI2, or NR4A3 genes, or any derivative DNA or RNA. Hybridization of an mRNA with the probe indicates that the IL-6, SELE, FOSB, NRK, NFKB2, FOXP3, ARG1, CEBPDP, TNFα, ADAMTS4, PENK, FOSL1, DUSP1, ACTA1, AGT, ATF3, CDK1, CXCL8, SELP, VCAN, TFPI2, or NR4A3 genes in question is being expressed.

In embodiments, the mRNA from the sample is immobilized on a solid surface and contacted with a probe, for example by running the isolated mRNA on an agarose gel and transferring the mRNA from the gel to a membrane, such as nitrocellulose. In other cases, the probes are immobilized on a solid surface and the mRNA is contacted with the probes, for example, in an Agilent gene chip array. A skilled artisan can readily adapt available mRNA detection methods for use in detecting the level of expression of the IL-6, SELE, FOSB, NRK, NFKB2, FOXP3, ARG1, CEBPDP, TNFα, ADAMTS4, PENK, FOSL1, DUSP1, ACTA1, AGT, ATF3, CDK1, CXCL8, SELP, VCAN, TFPI2, or NR4A3 genes.

An alternative method for determining the level of IL-6, SELE, FOSB, NRK, NFKB2, FOXP3, ARG1, CEBPDP, TNFα, ADAMTS4, PENK, FOSL1, DUSP1, ACTA1, AGT, ATF3, CDK1, CXCL8, SELP, VCAN, TFPI2, or NR4A3 gene expression in a sample involves the process of nucleic acid amplification of the IL-6, SELE, FOSB, NRK, NFKB2, FOXP3, ARG1, CEBPDP, TNFα, ADAMTS4, PENK, FOSL1, DUSP1, ACTA1, AGT, ATF3, CDK1, CXCL8, SELP, VCAN, TFPI2, or NR4A3 mRNA (or cDNA thereof), for example, by RT-PCR (U.S. Pat. No. 4,683,202), ligase chain reaction (Barany, Proc. Natl. Acad. Sci. USA 88:189-93, 1991), self-sustained sequence replication (Guatelli et al., Proc. Natl. Acad. Sci. USA 87:1874-78, 1990), transcriptional amplification system (Kwoh et al., Proc. Natl. Acad. Sci. USA 86:1173-77, 1989), Q-Beta Replicase (Lizardi et al., Bio/Technology 6:1197, 1988), rolling circle replication (U.S. Pat. No. 5,854,033), or any other nucleic acid amplification method, followed by the detection of the amplified molecules using available techniques. These detection schemes are especially useful for the detection of nucleic acid molecules if such molecules are present in very low numbers.

In some cases, IL-6, SELE, FOSB, NRK, NFKB2, FOXP3, ARG1, CEBPDP, TNFα, ADAMTS4, PENK, FOSL1, DUSP1, ACTA1, AGT, ATF3, CDK1, CXCL8, SELP, VCAN, TFPI2, or NR4A3 gene expression is assessed by quantitative RT-PCR. Numerous different PCR or QPCR protocols are available and can be directly applied or adapted for use using the IL-6, SELE, FOSB, NRK, NFKB2, FOXP3, ARG1, CEBPDP, TNFα, ADAMTS4, PENK, FOSL1, DUSP1, ACTA1, AGT, ATF3, CDK1, CXCL8, SELP, VCAN, TFPI2, or NR4A3 genes. Generally, in PCR, a target polynucleotide sequence is amplified by reaction with at least one oligonucleotide primer or pair of oligonucleotide primers. The primer(s) hybridize to a complementary region of the target nucleic acid and a DNA polymerase extends the primer(s) to amplify the target sequence. Under conditions sufficient to provide polymerase-based nucleic acid amplification products, a nucleic acid fragment of one size dominates the reaction products (the target polynucleotide sequence which is the amplification product). The amplification cycle is repeated to increase the concentration of the single target polynucleotide sequence. The reaction can be performed in any thermocycler commonly used for PCR. However, preferred are cyclers with real-time fluorescence measurement capabilities, for example, SMARTCYCLER® (Cepheid, Sunnyvale, Calif.), ABI PRISM 7700@(Applied Biosystems, Foster City, Calif.), ROTOR-GENE™ (Corbett Research, Sydney, Australia), LIGHTCYCLER® (Roche Diagnostics Corp, Indianapolis, Ind.), ICYCLER® (Biorad Laboratories, Hercules, Calif.) and MX4000@(Stratagene, La Jolla, Calif.).

Quantitative PCR (QPCR) (also referred as real-time PCR) may be used under some circumstances because it provides not only a quantitative measurement, but also reduced time and contamination. In some instances, the availability of full gene expression profiling techniques is limited due to requirements for fresh frozen tissue and specialized laboratory equipment, making the routine use of such technologies difficult in a clinical setting. However, QPCR gene measurement can be applied to standard formalin-fixed paraffin-embedded clinical tumor blocks, such as those used in archival tissue banks and routine surgical pathology specimens (Cronin et al. (2007) Clin Chem 53:1084-91)[Mullins 2007][Paik 2004]. As used herein, “quantitative PCR (or “real time QPCR”) refers to the direct monitoring of the progress of PCR amplification as it is occurring without the need for repeated sampling of the reaction products. In quantitative PCR, the reaction products may be monitored via a signaling mechanism (e.g., fluorescence) as they are generated and are tracked after the signal rises above a background level but before the reaction reaches a plateau. The number of cycles required to achieve a detectable or “threshold” level of fluorescence varies directly with the concentration of amplifiable targets at the beginning of the PCR process, enabling a measure of signal intensity to provide a measure of the amount of target nucleic acid in a sample in real time.

In some cases, microarrays are used for expression profiling. Microarrays are particularly well suited for this purpose because of the reproducibility between different experiments. DNA microarrays provide one method for the simultaneous measurement of the expression levels of large numbers of genes. Each array consists of a reproducible pattern of capture probes attached to a solid support. Labeled RNA or DNA is hybridized to complementary probes on the array and then detected by laser scanning. Hybridization intensities for each probe on the array are determined and converted to a quantitative value representing relative gene expression levels. See, for example, U.S. Pat. Nos. 6,040,138, 5,800,992 and 6,020,135, 6,033,860, and 6,344,316. High-density oligonucleotide arrays are particularly useful for determining the gene expression profile for a large number of RNAs in a sample. Techniques for the synthesis of these arrays using mechanical synthesis methods are described in, for example, U.S. Pat. No. 5,384,261. Although a planar array surface can be used, the array can be fabricated on a surface of virtually any shape or even a multiplicity of surfaces. Arrays can be nucleic acids (or peptides) on beads, gels, polymeric surfaces, fibers (such as fiber optics), glass, or any other appropriate substrate. See, for example, U.S. Pat. Nos. 5,770,358, 5,789,162, 5,708,153, 6,040,193 and 5,800,992. Arrays can be packaged in such a manner as to allow for diagnostics or other manipulation of an all-inclusive device. See, for example, U.S. Pat. Nos. 5,856,174 and 5,922,591.

When using microarray techniques, PCR amplified inserts of cDNA clones can be applied to a substrate in a dense array. The microarrayed genes, immobilized on the microchip, are suitable for hybridization under stringent conditions. Fluorescently labeled cDNA probes can be generated through incorporation of fluorescent nucleotides by reverse transcription of RNA extracted from tissues of interest. Labeled cDNA probes applied to the chip hybridize with specificity to each spot of DNA on the array. After stringent washing to remove non-specifically bound probes, the chip is scanned by confocal laser microscopy or by another detection method, such as a CCD camera. Quantitation of hybridization of each arrayed element allows for assessment of corresponding mRNA abundance.

With dual color fluorescence, separately labeled cDNA probes generated from two sources of RNA can be hybridized pairwise to the array. The relative abundance of the transcripts from the two sources corresponding to each specified gene is thus determined simultaneously. A miniaturized scale can be used for the hybridization, which provides convenient and rapid evaluation of the expression pattern for large numbers of genes. Such methods have been shown to have the sensitivity required to detect rare transcripts, which are expressed at a few copies per cell, and to reproducibly detect at least approximately two-fold differences in the expression levels (Schena et al., Proc. Natl. Acad. Sci. USA 93:106-49, 1996). Microarray analysis can be performed by commercially available equipment, following manufacturer's protocols, such as by using the Affymetrix GenChip technology, or Agilent ink jet microarray technology. The development of microarray methods for large-scale analysis of gene expression makes it possible to search systematically for molecular markers of cancer classification and outcome prediction in a variety of tumor types.

As used herein “level”, refers to a measure of the amount of, or a concentration of a transcription product, for instance an mRNA, or a translation product, for instance a protein or polypeptide.

As used herein “activity” refers to a measure of the ability of a transcription product or a translation product to produce a biological effect or to a measure of a level of biologically active molecules.

As used herein “expression level” further refer to gene expression levels or gene activity. Gene expression can be defined as the utilization of the information contained in a gene by transcription and translation leading to the production of a gene product.

The terms “increased,” or “increase” in connection with expression of the biomarkers described herein generally means an increase by a statically significant amount. For the avoidance of any doubt, the terms “increased” or “increase” means an increase of at least 10% as compared to a reference value, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference value or level, or at least about a 1.5-fold, at least about a 1.6-fold, at least about a 1.7-fold, at least about a 1.8-fold, at least about a 1.9-fold, at least about a 2-fold, at least about a 3-fold, or at least about a 4-fold, or at least about a 5-fold, at least about a 10-fold increase, any increase between 2-fold and 10-fold, at least about a 25-fold increase, or greater as compared to a reference level. In some embodiments, an increase is at least about 1.8-fold increase over a reference value.

Similarly, the terms “decrease,” or “reduced,” or “reduction,” or “inhibit” in connection with expression of the biomarkers described herein generally to refer to a decrease by a statistically significant amount. However, for avoidance of doubt, “reduced”, “reduction” or “decrease” or “inhibit” means a decrease by at least 10% as compared to a reference level, for example a decrease by at least about 20%, or at least about 30%, or at least about 40%, or at least about 50%, or at least about 60%, or at least about 70%, or at least about 80%, or at least about 90% or up to and including a 100% decrease (e.g. absent level or non-detectable level as compared to a reference sample), or any decrease between 10-100% as compared to a reference level.

A “reference value” is a predetermined reference level, such as an average or median of expression levels of each of IL-6, SELE, FOSB, NRK, NFKB2, FOXP3, ARG1, CEBPDP, TNFα, ADAMTS4, PENK, FOSL1, DUSP1, ACTA1, AGT, ATF3, CDK1, CXCL8, SELP, VCAN, TFPI2, or NR4A3 biomarkers in, for example, biological samples from a population of healthy subjects. The reference value can be an average or median of expression levels of each of IL-6, SELE, FOSB, NRK, NFKB2, FOXP3, ARG1, CEBPDP, TNFα, ADAMTS4, PENK, FOSL1, DUSP1, ACTA1, AGT, ATF3, CDK1, CXCL8, SELP, VCAN, TFPI2, or NR4A3 biomarkers in a chronological age group matched with the chronological age of the tested subject. In some embodiments, the reference biological samples can also be gender matched. In some embodiments, the reference biological samples can also be cancer containing tissue from a specific subgroup of patients, such as stage 1, stage 2, stage 3, or grade 1, grade 2, grade 3 cancers, non-metastatic cancers, untreated cancers, hormone treatment resistant cancers, or other relevant biological or prognostic subsets. For example, as explained herein, malignancy associated response signature expression levels in a sample can be assessed relative to normal prostate tissue from the same subject or from a sample from another subject or from a repository of normal subject samples. If the expression level of a biomarker is greater or less than that of the reference or the average expression level, the biomarker expression is said to be “increased” or “decreased,” respectively, as those terms are defined herein. Exemplary analytical methods for classifying expression of a biomarker, determining a malignancy associated response signature status, and scoring of a sample for expression of a malignancy associated response signature biomarker are explained in detail herein.

### Treatment

Methods are described herein for treating prostate cancer. Such methods can involve administering therapeutic agents that can treat prostate cancers with poor prognosis or a high risk of developing metastatic prostate cancer. Examples of such therapeutic agents can include one or more TNFα antagonists such as infliximab, adalimumab, etanercept, golimumab and certolizumab, adalimumab, certolizumab, erelzi, golimumab, or etanercept. Therapeutic agents can also include one or more immune checkpoint inhibitors (ICI) such as tecentriq, and libtayo, keytruda, opdivo, and yervoy. In embodiments, treatment for prostate cancer can be a combination therapy that can include administering one or more TNFα antagonists, SELE agonists, and ICI therapeutic agents combined with a chemotherapy agent (e.g. avastin).

As used herein, “solid tumor” is intended to include, but not be limited to, the following sarcomas and carcinomas: fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilms' tumor, cervical cancer, testicular tumor, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, melanoma, neuroblastoma, and retinoblastoma. Solid tumor is also intended to encompass epithelial cancers.

“Good prognosis” means that a patient is expected to have longer overall survival (OS), or progression-free survival (PFS), or disease-specific survival (DSS) or recurrence-free survival (RFS) compared to “poor prognosis” patients. These metrics are typically described by National Cancer Institute (NCI) as overall survival (OS), or progression-free survival (PFS) which is the length of time during and after the treatment of cancer, that a patient lives with the disease but it does not get worse, or disease-specific survival (DSS) that is the percentage of people in a treatment group who have not died from their cancer in a defined period of time, or recurrence-free survival (RFS) that is length of time after primary treatment for a cancer ends that the patient survives without any signs or symptoms of that cancer, also called as disease-free survival (DFS), or relapse-free survival (see website at cancer.gov/publications/dictionaries/cancer-terms/def/rfs).

“Poor prognosis” means that a patient is expected to have a shorter overall survival (OS), or progression-free survival (PFS), or disease-specific survival (DSS) or recurrence-free survival (RFS) compared to “good prognosis” patients.

### Kit

A kit is provided comprising at least one isolated probe that hybridizes to RNA for one or more of IL-6, SELE, FOSB, NRK, NFKB2, FOXP3, ARG1, CEBPDP, TNFα, ADAMTS4, PENK, FOSL1, DUSP1, ACTA1, AGT, ATF3, CDK1, CXCL8, SELP, VCAN, TFPI2, NR4A3, or any combination thereof, that is optionally bound to a solid support or at least one primer having a nucleotide sequence for detecting one or more of IL-6, SELE, FOSB, NRK, NFKB2, FOXP3, ARG1, CEBPDP, TNFα, ADAMTS4, PENK, FOSL1, DUSP1, ACTA1, AGT, ATF3, CDK1, CXCL8, SELP, VCAN, TFPI2, NR4A3, or any combination thereof. In one embodiment, the kit further comprises instructions for using the at least one probe or at least one primer in the method. In one embodiment, the solid support is selected from the group consisting of a bead, plate, membrane, array, or chip.

### Interleukin-6 (IL-6)

IL-6 is a cytokine with a wide variety of biological functions in immunity, tissue regeneration, and metabolism. The IL-6 gene is located on chromosome 7 (Gene ID: 3569; location NC_000007.14 (22727200 . . . 22731998). An example of an amino acid sequence for human IL-6 isoform 1 is available as UNIPROT accession no. P05231 and shown below as SEQ ID NO:1.

MNSFSTSAFGPVAFSLGLLLVLPAAFPAPVPPGEDSKDVAAPHRQPLIS
SERIDKQIRYILDGISALRKETCNKSNMCESSKEALAENNLNLPKMAEK
DGCFQSGFNEETCLVKIITGLLEFEVYLEYLQNRFESSEEQARAVQMST
KVLIQFLQKKAKNLDAITTPDPTTNASLLTKLQAQNQWLQDMTTHLILR
SFKEFLQSSLRALRQM

A cDNA sequence encoding the SEQ ID NO:1 IL-6 protein is available as NCBI accession no. NM_000600, shown below as SEQ ID NO:2.

| | |
|---|---|
| 1 | ATTCTGCCCT CGAGCCCACC GGGAACGAAA GAGAAGCTCT ATCTCCCCTC CAGGAGCCCA |
| 61 | GCTATGAACT CCTTCTCCAC AAGCGCCTTC GGTCCAGTTG CCTTCTCCCT GGGGCTGCTC |
| 121 | CTGGTGTTGC CTGCTGCCTT CCCTGCCCCA GTACCCCCAG GAGAAGATTC CAAAGATGTA |
| 181 | GCCGCCCCAC ACAGACAGCC ACTCACCTCT TCAGAACGAA TTGACAAACA AATTCGGTAC |
| 241 | ATCCTCGACG GCATCTCAGC CCTGAGAAAG GAGACATGTA ACAAGAGTAA CATGTGTGAA |
| 301 | AGCAGCAAAG AGGCACTGGC AGAAAACAAC CTGAACCTTC CAAAGATGGC TGAAAAAGAT |
| 361 | GGATGCTTCC AATCTGGATT CAATGAGGAG ACTTGCCTGG TGAAAATCAT CACTGGTCTT |
| 421 | TTGGAGTTTG AGGTATACCT AGAGTACCTC CAGAACAGAT TTGAGAGTAG TGAGGAACAA |
| 481 | GCCAGAGCTG TGCAGATGAG TACAAAAGTC CTGATCCAGT TCCTGCAGAA AAAGGCAAAG |
| 541 | AATCTAGATG CAATAACCAC CCCTGACCCA ACCACAAATG CCAGCCTGCT GACGAAGCTG |
| 601 | CAGGCACAGA ACCAGTGGCT GCAGGACATG ACAACTCATC TCATTCTGCG CAGCTTTAAG |
| 661 | GAGTTCCTGC AGTCCAGCCT GAGGGCTCTT CGGCAAATGT AGCATGGGCA CCTCAGATTG |
| 721 | TTGTTGTTAA TGGGCATTCC TTCTTCTGGT CAGAAACCTG TCCACTGGGC ACAGAACTTA |
| 781 | TGTTGTTCTC TATGGAGAAC TAAAAGTATG AGCGTTAGGA CACTATTTTA ATTATTTTTA |
| 841 | ATTTATTAAT ATTTAAATAT GTGAAGCTGA GTTAATTTAT GTAAGTCATA TTTATATTTT |
| 901 | TAAGAAGTAC CACTTGAAAC ATTTTATGTA TTAGTTTTGA AATAATAATG GAAAGTGGCT |
| 961 | ATGCAGTTTG AATATCCTTT GTTTCAGAGC CAGATCATTT CTTGGAAAGTG TAGGCTTAC |
| 1021 | CTCAAATAAA TGGCTAACTT ATACATATTT TTAAAGAAAT ATTTATATTG TATTTATATA |
| 1081 | ATGTATAAAT GGTTTTTATA CCAATAAATG GCATTTTAAA AAATTCA |

### E-Selectin (SELE)

SELE is a cell-surface glycoprotein having a role in immunoadhesion. SELE mediates in the adhesion of blood neutrophils in cytokine-activated endothelium through interaction with SELPLG/PSGL1. The SELE gene is located on chromosome 1 (Gene ID: 6401; location NC_000001 (169722640 . . . 169734079). An example of an amino acid sequence for human SELE is available as UNIPROT accession no. P16581 and shown below as SEQ ID NO:3.

MIASQFLSALTLVLLIKESGAWSYNTSTEAMTYDEASAYCQQRYTHLVAIQNKEEIEY
LNSILSYSPSYYWIGIRKVNNVWVWVGTQKPLTEEAKNWAPGEPNNRQKDEDCVEIYI
KREKDVGMWNDERCSKKKLALCYTAACTNTSCSGHGECVETINNYTCKCDPGFSGLKC
EQIVNCTALESPEHGSLVCSHPLGNFSYNSSCSISCDRGYLPSSMETMQCMSSGEWSA
PIPACNVVECDAVINPANGFVECFQNPGSFPWNTTCTFDCEEGFELMGAQSLQCTSSG
NWDNEKPTCKAVTCRAVRQPQNGSVRCSHSPAGEFTFKSSCNFTCEEGFMLQGPAQVE
CTTQGQWTQQIPVCEAFQCTALSNPERGYMNCLPSASGSFRYGSSCEFSCEQGFVLKG
SKRLQCGPTGEWDNEKPTCEAVRCDAVHQPPKGLVRCAHSPIGEFTYKSSCAFSCEEG
FELHGSTQLECTSQGQWTEEVPSCQVVKCSSLAVPGKINMSCSGEPVFGTVCKFACPE
GWTLNGSAARTCGATGHWSGLLPTCEAPTESNIPLVAGLSAAGLSLLTLAPFLLWLRK
CLRKAKKFVPASSCQSLESDGSYQKPSYIL

A cDNA sequence encoding the SEQ ID NO:3 SELE protein is available as NCBI accession no. NC_000001, shown below as SEQ ID NO:4.

| | |
|---|---|
| 1 | AGCTGTTCTT GGCTGACTTC ACATCAAAAC TCCTATACTG ACCTGAGACA GAGGCAGCAG |
| 61 | TGATACCCAC CTGAGAGATC CTGTGTTTGA ACAACTGCTT CCCAAAACGG AAAGTATTTC |
| 121 | AAGCCTAAAC CTTTGGGTGA AAAGAACTCT TGAAGTCATG ATTGCTTCAC AGTTTCTCTC |
| 181 | AGCTCTCACT TTGGTGCTTC TCATTAAAGA GAGTGGAGCC TGGTCTTACA ACACCTCCAC |
| 241 | GGAAGCTATG ACTTATGATG AGGCCAGTGC TTATTGTCAG CAAAGGTACA CACACCTGGT |
| 301 | TGCAATTCAA AACAAAGAAG AGATTGAGTA CCTAAACTCC ATATTGAGCT ATTCACCAAG |
| 361 | TTATTACTGG ATTGGAATCA GAAAAGTCAA CAATGTGTGG GTCTGGGTAG GAACCCAGAA |
| 421 | ACCTCTGACA GAAGAAGCCA AGAACTGGGC TCCAGGTGAA CCCAACAATA GGCAAAAAGA |
| 481 | TGAGGACTGC GTGGAGATCT ACATCAAGAG AGAAAAAGAT GTGGGCATGT GGAATGATGA |
| 541 | GAGGTGCAGC AAGAAGAAGC TTGCCCTATG CTACACAGCT GCCTGTACCA ATACATCCTG |
| 601 | CAGTGGCCAC GGTGAATGTG TAGAGACCAT CAATAATTAC ACTTGCAAGT GTGACCCTGG |
| 661 | CTTCAGTGGA CTCAAGTGTG AGCAAATTGT GAACTGTACA GCCCTGGAAT CCCCTGAGCA |
| 721 | TGGAAGCCTG GTTTGCAGTC ACCCACTGGG AAACTTCAGC TACAATTCTT CCTGCTCTAT |
| 781 | CAGCTGTGAT AGGGGTTACC TGCCAAGCAG CATGGAGACC ATGCAGTGTA TGTCCTCTGG |
| 841 | AGAATGGAGT GCTCCTATTC CAGCCTGCAA TGTGGTTGAG TGTGATGCTG TGACAAATCC |
| 901 | AGCCAATGGG TTCGTGGAAT GTTTCCAAAA CCCTGGAAGC TTCCCATGGA ACACAACCTG |
| 961 | TACATTTGAC TGTGAAGAAG GATTTGAACT AATGGGAGCC CAGAGCCTTC AGTGTACCTC |
| 1021 | ATCTGGGAAT TGGGACAACG AGAAGCCAAC GTGTAAAGCT GTGACATGCA GGGCCGTCCG |
| 1081 | CCAGCCTCAG AATGGCTCTG TGAGGTGCAG CCATTCCCCT GCTGGAGAGT TCACCTTCAA |
| 1141 | ATCATCCTGC GTGAGGAAGG CTTCATGTTG CAGGGACCAG AACTTCACCT CCCAGGTTGA |
| 1201 | ATGCACCACT GGACACAGCA AATCCCAGTT TGTGAAGCTT CAAGGGCAGT TCCAGTGCAC |
| 1261 | AGCCTTGTCC AACCCCGAGC GAGGCTACAT GAATTGTCTT CCTAGTGCTT CTGGCAGTTT |
| 1321 | CCGTTATGGG TCCAGCTGTG AGTTCTCCTG TGAGCAGGGT TTTGTGTTGA AGGGATCCAA |
| 1381 | AAGGCTCCAA TGTGGCCCCA CAGGGGAGTG GGACAACGAG AAGCCCACAT GTGAAGCTGT |
| 1441 | GAGATGCGAT GCTGTCCACC AGCCCCCGAA GGGTTTGGTG AGGTGTGCTC ATTCCCCTAT |
| 1501 | TGGAGAATTC ACCTACAAGT CCTCTTGTGC CTTCAGCTGT GAGGAGGGAT TTGAATTACA |
| 1561 | TGGATCAACT CAACTTGAGT GCACATCTCA GGGACAATGG ACAGAAGAGG TTCCTTCCTG |
| 1621 | CCAAGTGGTA AAATGTTCAA GCCTGGCAGT TCCGGGAAAG ATCAACATGA GCTGCAGTGG |
| 1681 | GGAGCCCGTG TTTGGCACTG TGTGCAAGTT CGCCTGTCCT GAAGGATGGA CGCTCAATGG |
| 1741 | CTCTGCAGCT CGGACATGTG GAGCCACAGG ACACTGGTCT GGCCTGCTAC CTACCTGTGA |
| 1801 | AGCTCCCACT GAGTCCAACA TTCCCTTGGT AGCTGGACTT TCTGCTGCTG GACTCTCCCT |
| 1861 | CCTGACATTA GCACCATTTC TCCTCTGGCT TCGGAAATGC TTACGGAAAG CAAAGAAATT |
| 1921 | TGTTCCTGCC AGCAGCTGCC AAAGCCTTGA ATCAGATGGA AGCTACCAAA AGCCTTCTTA |
| 1981 | CATCCTTTAA GTTCAAAAGA ATCAGAAACA GGTGCATCTG GGGAACTAGA GGGATACACT |
| 2041 | GAAGTTAACA GAGACAGATA ACTCTCCTCG GGTCTCTGGC CCTTCTTGCC TACTATGCCA |
| 2101 | GATGCCTTTA TGGCTGAAAC CGCAACACCC ATCACCACTT CAATAGATCA AAGTCCAGCA |
| 2161 | GGCAAGGACG GCCTTCAACT GAAAAGACTC AGTGTTCCCT TTCCTACTCT CAGGATCAAG |
| 2221 | AAAGTGTTGG CTAATGAAGG GAAAGGATAT TTTCTTCCAA GCAAAGGTGA AGAGACCAAG |
| 2281 | ACTCTGAAA TCTCAGAATTC CTTTTCTAAC TCTCCCTTGC TCGCTGTAAA ATCTTGGCAC |
| 2341 | AGAAACACAA TATTTTGTGG CTTTCTTTCT TTTGCCCTTC ACAGTGTTTC GACAGCTGAT |
| 2401 | TACACAGTTG CTGTCATAAG AATGAATAAT AATTATCCAG AGTTTAGAGG AAAAAAATGA |
| 2461 | CTAAAAATAT TATAACTTAA AAAAATGACA GATGTTGAAT GCCCACAGGC AAATGCATGG |
| 2521 | AGGGTTGTTA ATGGTGCAAA TCCTACTGAA TGCTCTGTGC GAGGGTTACT ATGCACAATT |
| 2581 | TAATCACTTT CATCCCTATG GGATTCAGTG CTTCTTAAAG AGTTCTTAAG GATTGTGATA |
| 2641 | TTTTTACTTG CATTGAATAT ATTATAATCT TCCATACTTC TTCATTCAAT ACAAGTGTGG |
| 2701 | TAGGGACTTA AAAAACTTGT AAATGCTGTC AACTATGATA TGGTAAAAGT TACTTATTCT |
| 2761 | AGATTACCCC CTCATTGTTT ATTAACAAAT TATGTTACAT CTGTTTTAAA TTTATTTCAA |
| 2821 | AAAGGGAAAC TATTGTCCCC TAGCAAGGCA TGATGTTAAC CAGAATAAAG TTCTGAGTGT |
| 2881 | TTTTACTACA GTTGTTTTTT GAAAACATGG TAGAATTGGA GAGTAAAAAC TGAATGGAAG |
| 2941 | GTTTGTATAT TGTCAGATAT TTTTTCAGAA ATATGTGGTT TCCACGATGA AAAACTTCCA |
| 3001 | TGAGGCCAAA CGTTTTGAAC TAATAAAAGC ATAAATGCAA ACACACAAAG GTATAATTTT |
| 3061 | ATGAATGTCT TTGTTGGAAA AGAATACAGA AAGATGGATG TGCTTTGCAT TCCTACAAAG |
| 3121 | ATGTTTGTCA GATATGATAT GTAAACATAA TTCTTGTATA TTATGGAAGA TTTTAAATTC |
| 3181 | ACAATAGAAA CTCACCATGT AAAAGAGTCA TCTGGTAGAT TTTTAACGAA TGAAGATGTC |
| 3241 | TAATAGTTAT TCCCTATTTG TTTTCTTCTG TATGTTAGGG TGCTCTGGAA GAGAGGAATG |
| 3301 | CCTGTGTGAG CAAGCATTTA TGTTTATTTA TAAGCAGATT TAACAATTCC AAAGGAATCT |
| 3361 | CCAGTTTTCA GTTGATCACT GGCAATGAAA AATTCTCAGT CAGTAATTGC CAAAGCTGCT |
| 3421 | CTAGCCTTGA GGAGTGTGAG AATCAAAACT CTCCTACACT TCCATTAACT TAGCATGTGT |
| 3481 | TGAAAAAAAA GTTTCAGAGA AGTTCTGGCT GAACACTGGC AACAACAAAG CCAACAGTCA |
| 3541 | AAACAGAGAT GTGATAAGGA TCAGAACAGC AGAGGTTCTT TTAAAGGGGC AGAAAAACTC |
| 3601 | TGGGAAATAA GAGAGAACAA CTACTGTGAT CAGGCTATGT ATGGAATACA GTGTTATTTT |
| 3661 | CTTTGAAATT GTTTAAGTGT TGTAAATATT TATGTAAACT GCATTAGAAA TTAGCTGTGT |
| 3721 | GAAATACCAG TGTGGTTTGT GTTTGAGTTT TATTGAGAAT TTTAAATTAT AACTTAAAAT |
| 3781 | ATTTTATAAT TTTTAAAGTA TATATTTATT TAAGCTTATG TCAGACCTAT TTGACATAAC |
| 3841 | ACTATAAAGG TTGACAATAA ATGTGCTTAT GTTTA |

### FOSB

FOSB protein heterodimerizes with proteins of the JUN family to form an AP-1 transcription factor complex, thereby enhancing their DNA binding activity to gene promoters containing an AP-1 consensus sequence 5′-TGA[GC]TCA-3′ and enhancing their transcriptional activity. The FOSB gene is located on chromosome 1 (Gene ID: 2354; location NC_000019.10 (45467996 . . . 45475179). An example of an amino acid sequence for human FOSB is available as UNIPROT accession no. P53539 and shown below as SEQ ID NO:5.

MFQAFPGDYDSGSRCSSSPSAESQYLSSVDSFGSPPTAAASQECAGLGE
MPGSFVPTVTAITTSQDLQWLVQPTLISSMAQSQGQPLASQPPVVDPYD
MPGTSYSTPGMSGYSSGGASGSGGPSTSGTTSGPGPARPARARPRRPRE
ETLTPEEEEKRRVRRERNKLAAAKCRNRRRELTDRLQAETDQLEEEKAE
LESEIAELQKEKERLEFVLVAHKPGCKIPYEEGPGPGPLAEVRDLPGSA
PAKEDGESWLLPPPPPPPLPFQTSQDAPPNLTASLFTHSEVQVLGDPFP
VVNPSYTSSFVLTCPEVSAFAGAQRTSGSDQPSDPLNSPSLLAL

A cDNA sequence encoding the SEQ ID NO:5 FOSB protein is available as NCBI accession no. NC 000019.10, shown below as SEQ ID NO:6.

| | |
|---|---|
| 1 | ATTCATAAGA CTCAGAGCTA CGGCCACGGC AGGGACACGC GGAACCAAGA CTTGGAAACT |
| 61 | TGATTGTTGT GGTTCTTCTT GGGGGTTATG AAATTTCATT AATCTTTTTT TTTCCGGGGA |
| 121 | GAAAGTTTTT GGAAAGATTC TTCCAGATAT TTCTTCATTT TCTTTTGGAG GACCGACTTA |
| 181 | CTTTTTTTGG TCTTCTTTAT TACTCCCCTC CCCCCGTGGG ACCCGCCGGA CGCGTGGAGG |
| 241 | AGACCGTAGC TGAAGCTGAT TCTGTACAGC GGGACAGCGC TTTCTGCCCC TGGGGGAGCA |
| 301 | ACCCCTCCCT CGCCCCTGGG TCCTACGGAG CCTGCACTTT CAAGAGGTAC AGCGGCATCC |
| 361 | TGTGGGGGCC TGGGCACCGC AGGAAGACTG CACAGAAACT TTGCCATTGT TGGAACGGGA |
| 421 | CGTTGCTCCT TCCCCGAGCT TCCCCGGACA GCGTACTTTG AGGACTCGCT CAGCTCACCG |
| 481 | GGGACTCCCA CGGCTCACCC CGGACTTGCA CCTTACTTCC CCAACCCGGC CATAGCCTTG |
| 541 | GCTTCCCGGC GACCTCAGCG TGGTCACAGG GGCCCCCCTG TGCCCAGGGA AATGTTTCAG |
| 601 | GCTTTCCCCG GAGACTACGA CTCCGGCTCC CGGTGCAGCT CCTCACCCTC TGCCGAGTCT |
| 661 | CAATATCTGT CTTCGGTGGA CTCCTTCGGC AGTCCACCCA CCGCCGCCGC CTCCCAGGAG |
| 721 | TGCGCCGGTC TCGGGGAAAT GCCCGGTTCC TTCGTGCCCA CGGTCACCGC GATCACAACC |
| 781 | AGCCAGGACC TCCAGTGGCT TGTGCAACCC ACCCTCATCT CTTCCATGGC CCAGTCCCAG |
| 841 | GGGCAGCCAC TGGCCTCCCA GCCCCCGGTC GTCGACCCCT ACGACATGCC GGGAACCAGC |
| 901 | TACTCCACAC CAGGCATGAG TGGCTACAGC AGTGGCGGAG CGAGTGGCAG TGGTGGGCCT |
| 961 | TCCACCAGCG GAACTACCAG TGGGCCTGGG CCTGCCCGCC CAGCCCGAGC CCGGCCTAGG |
| 1021 | AGACCCCGAG AGGAGACGCT CACCCCAGAG GAAGAGGAGA AGCGAAGGGT GCGCCGGGAA |
| 1081 | CGAAATAAAC TAGCAGCAGC TAAATGCAGG AACCGGCGGA GGGAGCTGAC CGACCGACTC |
| 1141 | CAGGCGGAGA CAGATCAGTT GGAGGAAGAA AAAGCAGAGC TGGAGTCGGA GATCGCCGAG |
| 1201 | CTCCAAAAGG AGAAGGAACG TCTGGAGTTT GTGCTGGTGG CCCACAAACC GGGCTGCAAG |
| 1261 | ATCCCCTACG AAGAGGGGCC CGGGCCGGGC CCGCTGGCGG AGGTGAGAGA TTTGCCGGGC |
| 1321 | TCAGCACCGG CTAAGGAAGA TGGCTTCAGC TGGCTGCTGC CGCCCCCGCC ACCACCGCCC |
| 1381 | CTGCCCTTCC AGACCAGCCA AGACGCACCC CCCAACCTGA CGGCTTCTCT CTTTACACAC |
| 1441 | AGTGAAGTTC AAGTCCTCGG CGACCCCTTC CCCGTTGTTA ACCCTTCGTA CACTTCTTCG |
| 1501 | TTTGTCCTCA CCTGCCCGGA GGTCTCCGCG TTCGCCGGCG CCCAACGCAC CAGCGGCAGT |
| 1561 | GACCAGCCTT CCGATCCCCT GAACTCGCCC TCCCTCCTCG CTCTGTGAAC TCTTTAGACA |
| 1621 | CACAAAACAA ACAAACACAT GGGGGAGAGA GACTTGGAAG AGGAGGAGGA GGAGGAGAAG |
| 1681 | GAGGAGAGAG AGGGGAAGAG ACAAAGTGGG TGTGTGGCCT CCCTGGCTCC TCCGTCTGAC |
| 1741 | CCTCTGCGGC CACTGCGCCA CTGCCATCGG ACAGGAGGAT TCCTTGTGTT TTGTCCTGCC |
| 1801 | TCTTGTTTCT GTGCCCCGGC GAGGCCGGAG AGCTGGTGAC TTTGGGGACA GGGGGTGGGA |
| 1861 | AGGGGATGGA CACCCCCAGC TGACTGTTGG CTCTCTGACG TCAACCCAAG CTCTGGGGAT |
| 1921 | GGGTGGGGAG GGGGGCGGGT GACGCCCACC TTCGGGCAGT CCTGTGTGAG GATTAAGGGA |
| 1981 | CGGGGGTGGG AGGTAGGCTG TGGGGTGGGC TGGAGTCCTC TCCAGAGAGG CTCAACAAGG |
| 2041 | AAAAATGCCA CTCCCTACCC AATGTCTCCC ACACCCACCC TTTTTTTGGG GTGCCTAGGT |
| 2101 | TGGTTTCCCC TGCACTCCCG ACCTTAGCTT ATTGATCCCA CATTTCCATG GTGTGAGATC |
| 2161 | CTCTTTACTC TGGGCAGAAG TGAGCCCCCC CCTTAAAGGG AATTCGATGC CCCCCTAGAA |
| 2221 | TAATCTCATC CCCCCACCCG ACTTCTTTTG AAATGTGAAC GTCCTTCCTT GACTGTCTAG |
| 2281 | CCACTCCCTC CCAGAAAAAC TGGCTCTGAT TGGAATTTCT GGCCTCCTAA GGCTCCCCAC |
| 2341 | CCCGAAATCA GCCCCCAGCC TTGTTTCTGA TGACAGTGTT ATCCCAAGAC CCTGCCCCCT |
| 2401 | GCCAGCCGAC CCTCCTGGCC TTCCTCGTTG GGCCGCTCTG ATTTCAGGCA GCAGGGGCTG |
| 2461 | CTGTGATGCC GTCCTGCTGG AGTGATTTAT ACTGTGAAAT GAGTTGGCCA GATTGTGGGG |
| 2521 | TGCAGCTGGG TGGGGCAGCA CACCTCTGGG GGGATAATGT CCCCACTCCC GAAAGCCTTT |
| 2581 | CCTCGGTCTC CCTTCCGTCC ATCCCCCTTC TTCCTCCCCT CAACAGTGAG TTAGACTCAA |
| 2641 | GGGGGTGACA GAACCGAGAA GGGGGTGACA GTCCTCCATC CACGTGGCCT CTCTCTCTCT |
| 2701 | CCTCAGGACC CTCAGCCCTG GCCTTTTTCT TTAAGGTCCC CCGACCAATC CCCAGCCTAG |
| 2761 | GACGCCAACT TCTCCCACCC CTTGGCCCCT CACATCCTCT CCAGGAAGGG AGTGAGGGGC |
| 2821 | TGTGACATTT TTCCGGAGAA GATTTCAGAG CTGAGGCTTT GGTACCCCCA AACCCCCAAT |
| 2881 | ATTTTTGGAC TGGCAGACTC AAGGGGCTGG AATCTCATGA TTCCATGCCC GAGTCCGCCC |
| 2941 | ATCCCTGACC ATGGTTTTGG CTCTCCCACC CCGCCGTTCC CTGCGCTTCA TCTCATGAGG |
| 3001 | ATTTCTTTAT GAGGCAAATT TATATTTTTT AATATCGGGG GGTGGACCAC GCCGCCCTCC |
| 3061 | ATCCGTGCTG CATGAAAAAC ATTCCACGTG CCCCTTGTCG CGCGTCTCCC ATCCTGATCC |
| 3121 | CAGACCCATT CCTTAGCTAT TTATCCCTTT CCTGGTTTCC GAAAGGCAAT TATATCTATT |
| 3181 | ATGTATAAGT AAATATATTA TATATGGATG TGTGTGTGTG CGTGCGCGTG AGTGTGTGAG |
| 3241 | CGCTTCTGCA GCCTCGGCCT AGGTCACGTT GGCCCTCAAA GCGAGCCGTT GAATTGGAAA |
| 3301 | CTGCTTCTAG AAACTCTGGC TCAGCCTGTC TCGGGCTGAC CCTTTTCTGA TCGTCTCGGC |
| 3361 | CCCTCTGATT GTTCCCGATG GTCTCTCTCC CTCTGTCTTT TCTCCTCCGC CTGTGTCCAT |
| 3421 | CTGACCGTTT TCACTTGTCT CCTTTCTGAC TGTCCCTGCC AATGCTCCAG CTGTCGTCTG |
| 3481 | ACTCTGGGTT CGTTGGGGAC ATGAGATTTT ATTTTTTGTG AGTGAGACTG AGGGATCGTA |
| 3541 | GATTTTTACA ATCTGTATCT TTGACAATTC TGGGTGCGAG TGTGAGAGTG TGAGCAGGGC |
| 3601 | TTGCTCCTGC CAACCACAAT TCAATGAATC CCCGACCCCC CTACCCCATG CTGTACTTGT |
| 3661 | GGTTCTCTTT TTGTATTTTG CATCTGACCC CGGGGGGCTG GGACAGATTG GCAATGGGCC |
| 3721 | GTCCCCTCTC CCCTTGGTTC TGCACTGTTG CCAATAAAAA GCTCTTAAAA ACGCA |

### Nik-Related Protein Kinase (NRK)

NRK is a serine/threonine kinase involved in the TNFα-induced signaling pathway. The NRK gene is located on chromosome X (Gene ID: 203447; location NC_000023.11 (105821786 . . . 105958610). An example of an amino acid sequence for human NRK is available as UNIPROT accession no. Q7Z2Y5 and shown below as SEQ ID NO:7.

MAGPGGWRDREVTDLGHLPDPTGIFSLDKTIGLGTYGRIYLGLHEKTGAFTAVKVMNA
RKTPLPEIGRRVRVNKYQKSVGWRYSDEEEDLRTELNLLRKYSFHKNIVSFYGAFFKL
SPPGQRHQLWMVMELCAAGSVTDVVRMTSNQSLKEDWIAYICREILQGLAHLHAHRVI
HRDIKGQNVLLTHNAEVKLVDFGVSAQVSRTNGRRNSFIGTPYWMAPEVIDCDEDPRR
SYDYRSDVWSVGITAIEMAEGAPPLCNLQPLEALFVILRESAPTVKSSGWSRKFHNEM
EKCTIKNFLFRPTSANMLQHPFVRDIKNERHVVESLTRHLTGIIKKRQKKGIPLIFER
EEAIKEQYTVRRFRGPSCTHELLRLPTSSRCRPLRVLHGEPSQPRWLPDREEPQVQAL
QQLQGAARVEMPLQALDSAPKPLKGQAQAPQRLQGAARVEMPLQAQVKAKASKPLQMQ
IKAPPRLRRAARVLMPLQAQVRAPRLLQVQSQVSKKQQAQTQTSEPQDLDQVPEEFQG
QDQVPEQQRQGQAPEQQQRHNQVPEQELEQNQAPEQPEVQEQAAEPAQUETEAEEPES
LRVNAQVFLPLLSQDHHVLLPLHLDTQVLIPVEGQTEGSPQAQAWTLEPPQAIGSVQA
LIEGLSRDLLRAPNSNNSKPLGPLQTLMENLSSNRFYSQPEQAREKKSKVSTLRQALA
KRLSPKRFRAKSSWRPEKLELSDLEARRQRRQRRWEDIFNQHEEELRQVDKDKEDESS
DNDEVFHSIQAEVQIEPLKPYISNPKKIEVQERSPSVPNNQDHAHHVKFSSSVPQRSL
LEQAQKPIDIRQRSSQNRQNWLAASESSSEEESPVTGRRSQSSPPYSTIDQKLLVDIH
VPDGFKVGKISPPVYLTNEWVGYNALSEIFRNDWLTPAPVIQPPEEDGDYVELYDASA
DTDGDDDDESNDTFEDTYDHANGNDDLDNQVDQANDVCKDHDDDNNKFVDDVNNNYYE
APSCPRASYGRDGSCKQDGYDGSRGKEEAYRGYGSHTANRSHGGSAASEDNAAIGDQE
EHAANIGSERRGSEGDGGKGVVRTSEESGALGLNGEENCSETDGPGLKRPASQDFEYL
QEEPGGGNEASNAIDSGAAPSAPDHESDNKDISESSTQSDESANHSSPSKGSGMSADA
NFASAILYAGFVEVPEESPKQPSEVNVNPLYVSPACKKPLIHMYEKEFTSEICCGSLW
GVNLLLGTRSNLYLMDRSGKADITKLIRRRPFRQIQVLEPLNLLITISGHKNRLRVYH
LTWLRNKILNNDPESKRRQEEMLKTEEACKAIDKLTGCEHFSVLQHEETTYIAIALKS
SIHLYAWAPKSFDESTAIKVCIDQSADSEGDYMSYQAYIRILAKIQAADPVNRFKRPD
ELLHLLKLKVFPTLDHKPVTVDLAIGSEKRLKIFFSSADGYHLIDAESEVMSDVTLPK
NPLEIIIPQNIIILPDCLGIGMMLTFNAEALSVEANEQLFKKILEMWKDIPSSIAFEC
TQRTTGWGQKAIEVRSLQSRVLESELKRRSIKKLRFLCTRGDKLFFTSTLRNHHSRVY
FMTLGKLEELQSNYDV

A cDNA sequence encoding the SEQ ID NO:7 NRK protein is available as NCBI accession no. NM 198465 XM 114703, shown below as SEQ ID NO:8.

| | |
|---|---|
| 1 | AGACACAGCG CTCTCGACAC GGAGCACCCT TCTAGCTTCT TCGTCTCCAG GACTGACGCT |
| 61 | CAGGCTCCTC TCTCGCCTTA GCCCAACTTG CTTTCCCGCC TCGCAAACTC CGGTTTCCCT |
| 121 | CCACTCCCAA CTCTTTTCAC TACACGTTTC CCCTCCTCTA TCTCCCACGC CACGAACCCC |
| 181 | GATCCCCAGA CTCCTCTCTC CCGCCCTCCT CCTTCCTCTC TCCTCCCTTC AACTCTTCAT |
| 241 | CCGCTTCCAC CTCAGACTCT GCGCGCACCC AATTCAGTCG CCCGCTCCCG TTCGGCTCCT |
| 301 | CGAAGCCATG GCGGGACCTG GGGGCTGGAG GGACAGGGAG GTCACGGATC TGGGCCACCT |
| 361 | GCCGGATCCA ACTGGAATAT TCTCACTAGA TAAAACCATT GGCCTTGGTA CTTATGGCAG |
| 421 | AATCTATTTG GGACTTCATG AGAAGACTGG TGCATTTACA GCTGTTAAAG TGATGAACGC |
| 481 | TCGTAAGACC CCTTTACCTG AAATAGGAAG GCGAGTGAGA GTGAATAAAT ATCAAAAATC |
| 541 | TGTTGGGTGG AGATACAGTG ATGAGGAAGA GGATCTCAGG ACTGAACTCA ACCTTCTGAG |
| 601 | GAAGTACTCT TTCCACAAAA ACATTGTGTC CTTCTATGGA GCATTTTTCA AGCTGAGTCC |
| 661 | CCCTGGTCAG CGGCACCAAC TTTGGATGGT GATGGAGTTA TGTGCAGCAG GTTCGGTCAC |
| 721 | TGATGTAGTG AGAATGACCA GTAATCAGAG TTTAAAAGAA GATTGGATTG CTTATATCTG |
| 781 | CCGAGAAATC CTTCAGGGCT TAGCTCACCT TCACGCACAC CGAGTAATTC ACCGGGACAT |
| 841 | CAAAGGTCAG AATGTGCTGC TGACTCATAA TGCTGAAGTA AAACTGGTTG ATTTTGGAGT |
| 901 | GAGTGCCCAG GTGAGCAGAA CTAATGGAAG AAGGAATAGT TTCATTGGGA CACCATACTG |
| 961 | GATGGCACCT GAGGTGATTG ACTGTGATGA GGACCCAAGA CGCTCCTATG ATTACAGAAG |
| 1021 | TGATGTGTGG TCTGTGGGAA TTACTGCCAT TGAAATGGCT GAAGGAGCCC CTCCTCTGTG |
| 1081 | TAACCTTCAA CCCTTGGAAG CTCTCTTCGT TATTTTGCGG GAATCTGCTC CCACAGTCAA |
| 1141 | ATCCAGCGGA TGGTCCCGTA AGTTCCACAA TTTCATGGAA AAGTGTACGA TAAAAAATTT |
| 1201 | CCTGTTTCGT CCTACTTCTG CAAACATGCT TCAACACCCA TTTGTTCGGG ATATAAAAAA |
| 1261 | TGAACGACAT GTTGTTGAGT CATTAACAAG GCATCTTACT GGAATCATTA AAAAAAGACA |
| 1321 | GAAAAAAGGA ATACCTTTGA TCTTTGAAAG AGAAGAAGCT ATTAAGGAAC AGTACACCGT |
| 1381 | GAGAAGATTC AGAGGACCCT CTTGCACTCA CGAGCTTCTG AGATTGCCAA CCAGCAGCAG |
| 1441 | ATGCAGACCA CTTAGAGTCC TGCATGGGGA ACCCTCTCAG CCAAGGTGGC TACCTGATCG |
| 1501 | AGAAGAGCCA CAGGTCCAGG CACTTCAGCA GCTACAGGGA GCAGCCAGGG TATTCATGCC |
| 1561 | ACTGCAGGCT CTGGACAGTG CACCTAAGCC TCTAAAGGGG CAGGCTCAGG CACCTCAACG |
| 1621 | ACTACAAGGG GCAGCTCGGG TGTTCATGCC ACTACAGGCT CAGGTGAAGG CTAAGGCCTC |
| 1681 | TAAACCTCTA CAAATGCAGA TTAAGGCACC TCCACGACTA CGGAGGGCAG CCAGGGTGCT |
| 1741 | CATGCCACTA CAGGCACAGG TTAGGGCACC TAGGCTTCTG CAGGTACAGT CCCAGGTATC |
| 1801 | CAAAAAGCAG CAGGCCCAGA CCCAGACATC AGAACCACAA GATTTGGACC AGGTACCAGA |
| 1861 | GGAATTTCAG GGTCAAGATC AGGTACCCGA ACAACAAAGG CAGGGCCAGG CCCCTGAACA |
| 1921 | ACAGCAGAGG CACAACCAGG TGCCTGAACA AGAGCTGGAG CAGAACCAGG CACCTGAACA |
| 1981 | GCCAGAGGTA CAGGAACAGG CTGCCGAGCC TGCACAGGCA GAGACTGAGG CAGAGGAACC |
| 2041 | TGAGTCATTA CGAGTAAATG CCCAGGTATT TCTGCCCCTG CTATCACAAG ATCACCATGT |
| 2101 | GCTGTTGCCA CTACATTTGG ATACTCAGGT GCTCATTCCA GTAGAGGGGC AAACTGAAGG |
| 2161 | ATCACCTCAG GGACACTAGA ACCCCCACAG GCAATTGGCT GCACAGGCTT CAGTTCAAGC |
| 2221 | ACTGATAGAG GGACTATCAA GAGACTTGCT TCGGGCACCA AACTCAAATA ACTCAAAGCC |
| 2281 | ACTTGGTCCG TTGCAAACCC TGATGGAAAA TCTGTCATCA AATAGGTTTT ACTCACAACC |
| 2341 | AGAACAGGCA CGGGAGAAAAAATCAAAAGT TTCTACTCTG AGGCAAGCAC TGGCAAAAAG |
| 2401 | ACTATCACCA AAGAGGTTCA GGGCAAAGTC ATCATGGAGA CCTGAAAAGC TTGAACTCTC |
| 2461 | GGATTTAGAA GCCCGCAGGC AAAGGCGCCA ACGCAGATGG GAAGATATCT TTAATCAGCA |
| 2521 | TGAGGAAGAA TTGAGACAAG TTGATAAAGA CAAAGAAGAT GAATCATCAG ACAATGATGA |
| 2581 | AGTATTTCAT TCGATTCAGG CTGAAGTCCA GATAGAGCCA TTGAAGCCAT ACATTTCAAA |
| 2641 | TCCTAAAAAA ATTGAGGTTC AAGAGAGATC TCCTTCTGTG CCTAACAACC AGGATCATGC |
| 2701 | ACATCATGTC AAGTTCTCTT CAAGCGTTCC TCAGCGGTCT CTTTTGGAAC AAGCTCAGAA |
| 2761 | GCCCATTGAC ATCAGACAAA GGAGTTCGCA AAATCGTCAA AATTGGCTGG CAGCATCAGA |
| 2821 | ATCTTCTTCT GAGGAAGAAA GTCCTGTGAC TGGAAGGAGG TCTCAGTCAT CACCACCTTA |
| 2881 | TTCTACTATT GATCAGAAGT TGCTGGTTGA CATCCATGTT CCAGATGGAT TTAAAGTAGG |
| 2941 | AAAAATATCA CCCCCTGTAT ACTTGACAAA CGAATGGGTA GGCTATAATG CACTCTCTGA |
| 3001 | AATCTTCCGG AATGATTGGT TAACTCCGGC ACCTGTCATT CAGCCACCTG AAGAGGATGG |
| 3061 | TGATTATGTT GAACTCTATG ATGCCAGTGC TGATACTGAT GGTGATGATG ATGATGAGTC |
| 3121 | TAATGATACT TTTGAAGATA CCTATGATCA TGCCAATGGC AATGATGACT TGGATAACCA |
| 3181 | GGTTGATCAG GCTAATGATG TTTGTAAAGA CCATGATGAT GACAACAATA AGTTTGTTGA |
| 3241 | TGATGTAAAT AATAATTATT ATGAGGCGCC TAGTTGTCCA AGGGCAAGCT ATGGCAGAGA |
| 3301 | TGGAAGCTGC AAGCAAGATG GTTATGATGG AAGTCGTGGA AAAGAGGAAG CCTACAGAGG |
| 3361 | CTATGGAAGC CATACAGCCA ATAGAAGCCA TGGAGGAAGT GCAGCCAGTG AGGACAATGC |
| 3421 | AGCCATTGGA GATCAGGAAG AACATGCAGC CAATATAGGC AGTGAAAGAA GAGGCAGTGA |
| 3481 | GGGTGATGGA GGTAAGGGAG TCGTTCGAAC CAGTGAAGAG AGTGGAGCCC TTGGACTCAA |
| 3541 | TGGAGAAGAA AATTGCTCAG AGACAGATGG TCCAGGATTG AAGAGACCTG CGTCTCAGGA |
| 3601 | CTTTGAATAT CTACAGGAGG AGCCAGGTGG TGGAAATGAG GCCTCAAATG CCATTGACTC |
| 3661 | AGGTGCTGCA CCGTCAGCAC CTGATCATGA GAGTGACAAT AAGGACATAT CAGAATCATC |
| 3721 | AACACAATCA GATTTTTCTG CCAATCACTC ATCTCCTTCC AAAGGTTCTG GGATGTCTGC |
| 3781 | TGATGCTAAC TTTGCCAGTG CCATCTTATA CGCTGGATTC GTAGAAGTAC CTGAGGAATC |
| 3841 | ACCTAAGCAA CCCTCTGAAG TCAATGTTAA CCCACTCTAT GTCTCTCCTG CATGTAAAAA |
| 3901 | ACCACTAATC CACATGTATG AAAAGGAGTT CACTTCTGAG ATCTGCTGTG GTTCTTTGTG |
| 3961 | GGGAGTCAAT TTGCTGTTGG GAACCCGATC TAATCTATAT CTGATGGACA GAAGTGGAAA |
| 4021 | GGCTGACATT ACTAAACTTA TAAGGCGAAG ACCATTCCGC CAGATTCAAG TCTTAGAGCC |
| 4081 | ACTCAATTTG CTGATTACCA TCTCAGGTCA TAAGAACAGA CTTCGGGTGT ATCATCTGAC |
| 4141 | AACAAGATTT TGAATAATGA TCCAGAAAGT AAAAGAAGGC CTGGTTGAGG AAGAAGAAAT |
| 4201 | GCTGAAGACA GAGGAAGCCT GCAAAGCTAT TGATAAGTTA ACAGGCTGTG AACACTTCAG |
| 4261 | TGTCCTCCAA CATGAAGAAA CAACATATAT TGCAATTGCT TTGAAATCAT CAATTCACCT |
| 4321 | TTATGCATGG GCACCAAAGT CCTTTGATGA AAGCACTGCT ATTAAAGTAT GCATTGATCA |
| 4381 | ATCAGCAGAC TCTGAAGGAG ACTACATGTC CTATCAAGCC TATATACGAA TACTGGCAAA |
| 4441 | AATACAGGCA GCTGATCCAG TGAACCGGTT TAAGAGACCA GATGAGCTCC TTCATTTGCT |
| 4501 | GAAGCTCAAG GTATTTCCAA CACTTGATCA TAAGCCAGTG ACAGTTGACC TGGCTATTGG |
| 4561 | TTCTGAAAAA AGACTAAAGA TTTTCTTCAG CTCAGCAGAT GGATATCACC TCATCGATGC |
| 4621 | AGAATCTGAG GTTATGTCTG ATGTGACCCT GCCAAAGAAT CCCCTGGAAA TCATTATACC |
| 4681 | ACAGAATATC ATCATTTTAC CTGATTGCTT GGGAATTGGC ATGATGCTCA CCTTCAATGC |
| 4741 | TGAAGCCCTC TCTGTGGAAG CAAATGAACA ACTCTTCAAG AAGATCCTTG AAATGTGGAA |
| 4801 | AGACATACCA TCTTCTATAG CTTTTGAATG TACACAGCGA ACCACAGGAT GGGGCCAAAA |
| 4861 | GGCCATTGAA GTGCGCTCTT TGCAATCCAG GGTTCTGGAA AGTGAGCTGA AGCGCAGGTC |
| 4921 | AATTAAGAAG CTGAGATTCC TGTGCACCCG GGGTGACAAG CTGTTCTTTA CCTCTACCCT |
| 4981 | GCGCAATCAC CACAGCCGGG TTTACTTCAT GACACTTGGA AAACTTGAAG AGCTCCAAAG |
| 5041 | CAATTATGAT GTCTAAAAGT TTCCAGTGAT TTATTACCAC ATTATAAACA TCATGTATAG |
| 5101 | GCAGTCTGCA TCTTCAGATT TCAGAGATTA AATGAGTATT CAGTTTTATT TTTAGTAAAG |
| 5161 | ATTAAATCCA AAACTTTACT TTTAATGTAG CACAGAATAG TTTTAATGAG AAATGCAGCT |
| 5221 | TTATGTATAA AATTAACTAT AGCAAGCTCT AGGTACTCCA ATGGTGTACA ATGTCTTTTG |
| 5281 | CACAAACTTT GTAACTTTTG TTACTGTGAA TTCAAACATT ACTCTTTGGA CAGTTTGGAC |
| 5341 | AGTATCTGTA TTCAGATTTT ACAACATGGA GTAAAGAAAC CTGTTATGAA TTAGATTACA |
| 5401 | AGCAGCCTTC AAAAGAATTG GCACTGGGAT AAGATTTTTC AGAAAAAGAA AAACATCGGC |
| 5461 | AAACTGTGTG TGATTTTTCC AAAGCTATAT AAAGAACCAA AGGTTTAGTC AAGAAACAAA |
| 5521 | AATCTTAAAG ATTATTATAA CCCAGACTAA GGTTGAACAA CCTGCATGCC CAGAGAAAAC |
| 5581 | TATGGCGACA AAGGGGAAAA GGCCACCACT CGTTTTCTCA CTGATTCATG CCAATTAAGC |
| 5641 | CTACAGTTAA AGACCAGTTT TGTTCTTTTC ACCCATTTTT AAGCTGGTTT TCTCCTGATA |
| 5701 | AGAAGAAAGG AAGAAAGCCC CAGACGCTTG GTTTTTCTCA GAACCCCCAA AAGATGTGCA |
| 5761 | ATAGCTGTTG TTACAAACCA CCAAATAATA CAGTTGTGAG CCTGAATACA GGACTGAACT |
| 5821 | CCTATACACG TGTACTGTAG AATGAGTATT TTTTAATACC TTAAGGTAGG CGTCAAATTC |
| 5881 | TACTCCCCAA AGCAGAGATA GATTGATTTA TCAAAATTAT TATCTGGCCA ACAGTGTGAC |
| 5941 | TATCAGACAG CATCAAATAT TTGCCCAATC CAAGATTAGA CTACACAAAA GCTTCCTTCC |
| 6001 | AGTATTAAAC AAAAAGAATT AAACATAACT ATGAAAAAAC TTTGCTAATA TCTGTGTTTT |
| 6061 | TCAGATTTCA TTTTTTGTAA AATCAGAAAT TAATCTAAAC ATATTCAGTG ATAAGTTCAT |
| 6121 | GTGTAACGAC TTAATGTTAA AGGTTAAAAA AAAGATTTCA CAAAATATAC AACTTTCACC |
| 6181 | ATATATATAA GCCTGCAAAA TTAGAGTAGT GAAAGTCATG CTAGTCCATC ACCCAAATAT |
| 6241 | GTTATAGACG CCATAGACAG GTGATGTTTG GTCACCTATG GTAACTGCTA CCTGATGAAG |
| 6301 | AGCATAATTT CTGCATATCC ATCCTCAATA CCATGGTAAA TTCTGGGGCA ATAGAGAAGC |
| 6361 | AACAGAACTG CCACAAAGTA TACCTCAATA TAATTCCTCT AGTTCTGCTT CTAAAATCTG |
| 6421 | AGGACAGTGC TAGTGGGAAA ATAATTTTCA AACTACCTGG TTAACCAAAA TACAAAAGCA |
| 6481 | GCTGACTATG TGTGATTTCA TAATAGCACA TTTCTTGACA CTTAGTGCTA GAAATGAAGA |
| 6541 | TTTGGATTTT CCTAACAACT TACATCAAGA ATGTAGTGTA GCTCATTATT GAGAATTTAG |
| 6601 | GAAAGCCTGA ATCCATTAAT TAAGGAAATA AATGTGACTC ACATTTCTTT TACTGTGACA |
| 6661 | CAATAATGTG ATCCTAAAAC TGGCTTATCC TTGAGTGTTT ACAACTCAAA CAACTTTTTG |
| 6721 | AATGCAGTAG TTTTTTTTTT TTAAAAACAA ACTTTTATGT CAAATTTTTT TTCTTAGAAG |
| 6781 | TAGTCTTCAT TATTATAAAT TTGTACACCA AAAGGCCATG GGGAACTTTG TGCAAGTACC |
| 6841 | TCATCGCTGA GCAAATGGAG CTTGCTATGT TTTAATTTCA GAAAATTTCC TCATATACGT |
| 6901 | AGTGTGTAGA ATCAAGTCTT TTAATAATTC ATTTTTTCTT CATAATATTT ACTCAAAGTT |
| 6961 | AAGCTTAAAA ATAAGTTTTA TCTTAAAATC ATATTTGAAG ACAGTAAGAC AGTAAACTAT |
| 7021 | TTTAGGAAGT CAACCCCCAT TGCACTCTGT GGCAGTTATT CTGGTAAAAA TAGGCAAAAG |
| 7081 | TGACCTGAAT CTACAATGAT GTCCCAAAGT AACCAAGTAA GAGAGATTGT AAATGATAAA |
| 7141 | CCGAGCTTTA AAGGATAAAG TGTTAATAAA GAAAGGAAGC TGGGCACATG TCAAAAAGGG |
| 7201 | AGATCGAAAT GTTAGGTAAT CATTTAGAAA GGACAGAAAA TATTTAAAGT GGCTCATAGG |
| 7261 | TAATGAATAT TTCTGACTTA GATGTAAATC CATCTGGAAT CTTTACATCC TTTGCCAGCT |
| 7321 | GAAACAAGAA AGTGAAGGGA CAATGATATT TCATGGTCAG TTTATTTTGT AAGAGACAGA |
| 7381 | AGAAATTATA TCTATACATT ACCTTGTAGC AGCAGTACCT GGAAGCCCCA GCCCGTCACA |
| 7441 | GAAGTGTGGA GGGGGGCTCC TGACTAGACA ATTTCCCTAG CCCTTGTGAT TTGAAGCATG |
| 7501 | AAAGTTCTGG CAGGTTATGA GCAGCACTAG GGATAAAGTA TGGTTTTATT TTGGTGTAAT |
| 7561 | TTAGGTTTTT CAACAAAGCC CTTGTCTAAA ATAAAAGGCA TTATTGGAAA TATTTGAAAA |
| 7621 | CTAGAAAATG ATGGATAAAA GGGCTGATAA GAAAATTTCT GACTGTCAGT AGAAGTGAGA |
| 7681 | TAAGATCCTC AGAGGAAACA GTAAGAAGGG ATAATCATTA AGATAGTAAA ACAGGCAAAG |
| 7741 | CAGAATCACA TGTGCACACA CACATACACA TGTAAACATT GGAATGCATA AGTTTTAATA |
| 7801 | TTTTAGCGCT ATCAGTTTCT AAATGCATTA ATTACTAACT GCCCTCTCCC AAGATTCATT |
| 7861 | TAGTTCAAAC AGTATCCGTA AACTAGGAAT AATGCCACAT GCATTCAATG GGATCTTTTA |
| 7921 | AGTACTCTTC AGTTTGTTCC AAGAAATGTG CCTACTGAAA TCAAATTAAT TTGTATTCAA |
| 7981 | TGTGTACTTC AAGACTGCTA ATTGTTTCAT CTGAAAGCCT ACAATGAATC ATTGTTCAAC |
| 8041 | CTTGAAAAAT AAAATTTTGT AAAACA |

### Nuclear Factor-Kappa-B (NF-κB2)

NF-κB2 is a pleiotropic transcription factor present in almost all cell types and is the endpoint of a series of signal transduction events that are initiated by a vast array of stimuli related to many biological processes such as inflammation, immunity, differentiation, cell growth, tumorigenesis and apoptosis. The NF-κB2 gene is located on chromosome 10 (Gene ID: 4791; location NC_000010 (102394110 . . . 102402529). An example of an amino acid sequence for human NF-κB2 is available as UNIPROT accession no. Q00653 and shown below as SEQ ID NO:9.

MESCYNPGLDGIIEYDDFKLNSSIVEPKEPAPETADGPYLVIVEQPKQRGFRFRYGCE
GPSHGGLPGASSEKGRKTYPTVKICNYEGPAKIEVDLVTHSDPPRAHAHSLVGKQCSE
LGICAVSVGPKDMTAQFNNLGVLHVTKKNMMGTMIQKLQRQRLRSRPQGLTEAEQREL
EQEAKELKKVMDLSIVRLRFSAFLRASDGSFSLPLKPVISQPIHDSKSPGASNLKISR
MDKTAGSVRGGDEVYLLCDKVQKDDIEVRFYEDDENGWQAFGDFSPTDVHKQYAIVER
TPPYHKMKIERPVTVFLQLKRKRGGDVSDSKQFTYYPLVEDKEEVQRKRRKALPTFSQ
PFGGGSHMGGGSGGAAGGYGGAGGGGSLGFFPSSLAYSPYQSGAGPMGCYPGGGGGAQ
MAATVPSRDSGEEAAEPSAPSRTPQCEPQAPEMLQRAREYNARLFGLAQRSARALLDY
GVTADARALLAGQRHLLTAQDENGDTPLHLAIIHGQTSVIEQIVYVIHHAQDLGVVNL
TNHLHQTPLHLAVITGQTSVVSFLLRVGADPALLDRHGDSAMHLALRAGAGAPELLRA
LLQSGAPAVPQLLHMPDFEGLYPVHLAVRARSPECLDLLVDSGAEVEATERQGGRTAL
HLATEMEELGLVTHLVTKLRANVNARTFAGNTPLHLAAGLGYPTLTRLLLKAGADIHA
ENEEPLCPLPSPPTSDSDSDSEGPEKDTRSSFRGHTPLDLTCSTKVKTLLLNAAQNTM
EPPLTPPSPAGPGLSLGDTALQNLEQLLDGPEAQGSWAELAERLGLRSLVDTYRQTTS
PSGSLLRSYELAGGDLAGLLEALSDMGLEEGVRLLRGPETRDKLPSTAEVKEDSAYGS
QSVEQEAEKLGPPPEPPGGLCHGHPQPQVH

A cDNA sequence encoding the SEQ ID NO:9 NF-κB2 protein is available as NCBI accession no. NM_001077494, shown below as SEQ ID NO:10.

| | |
|---|---|
| 1 | CCGCAACCAG AGCCGCCGCC ACGGTGAGTG GCTGGATTCA GACCCCTGGG TGGCCGGGAC |
| 61 | AAGAGAAAAG AGGGAGGAGG GCCTTTAGCG GACAGCGCCT GGGGCTGGAG AGCAGCAGCT |
| 121 | GCACACAGCC GGAAAGGGCG CGCAGGCGAC GACACTCGGA TCCACGTCGA CACCGTTGTA |
| 181 | CAAAGATACG CGGACCCGCG GGCGTCTAAA ATTCTGGGAA GCAGAACCTG GCCGGAGCCA |
| 241 | CTAGACAGAG CCGGGCCTAG CCCAGAGACA TGGAGAGTTG CTACAACCCA GGTCTGGATG |
| 301 | GTATTATTGA ATATGATGAT TTCAAATTGA ACTCCTCCAT TGTGGAACCC AAGGAGCCAG |
| 361 | CCCCAGAAAC AGCTGATGGC CCCTACCTGG TGATCGTGGA ACAGCCTAAG CAGAGAGGCT |
| 421 | TCCGATTTCG ATATGGCTGT GAAGGCCCCT CCCATGGAGG ACTGCCCGGT GCCTCCAGTG |
| 481 | AGAAGGGCCG AAAGACCTAT CCCACTGTCA AGATCTGTAA CTACGAGGGA CCAGCCAAGA |
| 541 | TCGAGGTGGA CCTGGTAACA CACAGTGACC CACCTCGTGC TCATGCCCAC AGTCTGGTGG |
| 601 | GCAAGCAATG CTCGGAGCTG GGGATCTGCG CCGTTTCTGT GGGGCCCAAG GACATGACTG |
| 661 | CCCAATTTAA CAACCTGGGT GTCCTGCATG TGACTAAGAA GAACATGATG GGGACTATGA |
| 721 | TACAAAAACT TCAGAGGCAG CGGCTCCGCT CTAGGCCCCA GGGCCTTACG GAGGCCGAGC |
| 781 | AGCGGGAGCT GGAGCAAGAG GCCAAAGAAC TGAAGAAGGT GATGGATCTG AGTATAGTGC |
| 841 | GGCTGCGCTT CTCTGCCTTC CTTAGAGCCA GTGATGGCTC CTTCTCCCTG CCCCTGAAGC |
| 901 | CAGTCATCTC CCAGCCCATC CATGACAGCA AATCTCCGGG GGCATCAAAC CTGAAGATTT |
| 961 | CTCGAATGGA CAAGACAGCA GGCTCTGTGC GGGGTGGAGA TGAAGTTTAT CTGCTTTGTG |
| 1021 | ACAAGGTGCA GAAAGATGAC ATTGAGGTTC GGTTCTATGA GGATGATGAG AATGGATGGC |
| 1081 | AGGCCTTTGG GGACTTCTCT CCCACAGATG TGCATAAACA GTATGCCATT GTGTTCCGGA |
| 1141 | CACCCCCCTA TCACAAGATG AAGATTGAGC GGCCTGTAAC AGTGTTTCTG CAACTGAAAC |
| 1201 | GCAAGCGAGG AGGGGACGTG TCTGATTCCA AACAGTTCAC CTATTACCCT CTGGTGGAAG |
| 1261 | ACAAGGAAGA GGTGCAGCGG AAGCGGAGGA AGGCCTTGCC CACCTTCTCC CAGCCCTTCG |
| 1321 | GGGGTGGCTC CCACATGGGT GGAGGCTCTG GGGGTGCAGC CGGGGGCTAC GGAGGAGCTG |
| 1381 | GAGGAGGTGG CAGCCTCGGT TTCTTCCCCT CCTCCCTGGC CTACAGCCCC TACCAGTCCG |
| 1441 | GCGCGGGCCC CATGGGCTGC TACCCGGGAG GCGGGGGCGG GGCGCAGATG GCCGCCACGG |
| 1501 | TGCCCAGCAG GGACTCCGGG GAGGAAGCCG CGGAGCCGAG CGCCCCCTCC AGGACCCCCC |
| 1561 | AGTGCGAGCC GCAGGCCCCG GAGATGCTGC AGCGAGCTCG AGAGTACAAC GCGCGCCTGT |
| 1621 | TCGGCCTGGC GCAGCGCAGC GCCCGAGCCC TACTCGACTA CGGCGTCACC GCGGACGCGC |
| 1681 | GCGCGCTGCT GGCGGGACAG CGCCACCTGC TGACGGCGCA GGACGAGAAC GGAGACACAC |
| 1741 | CACTGCACCT AGCCATCATC CACGGGCAGA CCAGTGTCAT TGAGCAGATA GTCTATGTCA |
| 1801 | TCCACCACGC CCAGGACCTC GGCGTTGTCA ACCTCACCAA CCACCTGCAC CAGACGCCCC |
| 1861 | TGCACCTGGC GGTGATCACG GGGCAGACGA GTGTGGTGAG CTTTCTGCTG CGGGTAGGTG |
| 1921 | CAGACCCAGC TCTGCTGGAT CGGCATGGAG ACTCAGCCAT GCATCTGGCG CTGCGGGCAG |
| 1981 | GCGCTGGTGC TCCTGAGCTG CTGCGTGCAC TGCTTCAGAG TGGAGCTCCT GCTGTGCCCC |
| 2041 | AGCTGTTGCA TATGCCTGAC TTTGAGGGAC TGTATCCAGT ACACCTGGCG GTCCGAGCCC |
| 2101 | GAAGCCCTGA GTGCCTGGAT CTGCTGGTGG ACAGTGGGGC TGAAGTGGAG GCCACAGAGC |
| 2161 | GGCAGGGGGG ACGAACAGCC TTGCATCTAG CCACAGAGAT GGAGGAGCTG GGGTTGGTCA |
| 2221 | CCCATCTGGT CACCAAGCTC CGGGCCAACG TGAACGCTCG CACCTTTGCG GGAAACACAC |
| 2281 | CCCTGCACCT GGCAGCTGGA CTGGGGTACC CGACCCTCAC CCGCCTCCTT CTGAAGGCTG |
| 2341 | GTGCTGACAT CCATGCTGAA AACGAGGAGC CCCTGTGCCC ACTGCCTTCA CCCCCTACCT |
| 2401 | CTGATAGCGA CTCGGACTCT GAAGGGCCTG AGAAGGACAC CCGAAGCAGC TTCCGGGGCC |
| 2461 | ACACGCCTCT TGACCTCACT TGCAGCACCA AGGTGAAGAC CTTGCTGCTA AATGCTGCTC |
| 2521 | AGAACACCAT GGAGCCACCC CTGACCCCGC CCAGCCCAGC AGGGCCGGGA CTGTCACTTG |
| 2581 | GTGATACAGC TCTGCAGAAC CTGGAGCAGC TGCTAGACGG GCCAGAAGCC CAGGGCAGCT |
| 2641 | GGGCAGAGCT GGCAGAGCGT CTGGGGCTGC GCAGCCTGGT AGACACGTAC CGACAGACAA |
| 2701 | CCTCACCCAG TGGCAGCCTC CTGCGCAGCT ACGAGCTGGC TGGCGGGGAC CTGGCAGGTC |
| 2761 | TACTGGAGGC CCTGTCTGAC ATGGGCCTAG AGGAGGGAGT GAGGCTGCTG AGGGGTCCAG |
| 2821 | AAACCCGAGA CAAGCTGCCC AGCACAGCAG AGGTGAAGGA AGACAGTGCG TACGGGAGCC |
| 2881 | AGTCAGTGGA GCAGGAGGCA GAGAAGCTGG GCCCACCCCC TGAGCCACCA GGAGGGCTCT |
| 2941 | GCCACGGGCA CCCCCAGCCT CAGGTGCACT GACCTGCTGC CTGCCCCCAG CCCCCTTCCC |
| 3001 | GGACCCCCTG TACAGCGTCC CCACCTATTT CAAATCTTAT TTAACACCCC ACACCCACCC |
| 3061 | CTCAGTTGGG ACAAATAAAG GATTCTCATG GGAAGGGGAG GACCCCTCCT TCCCAACTTA |
| 3121 | TGGCA |

Tumor necrosis factor (TNFα)

TNFα is a cytokine with important functions as a pathological component of autoimmune diseases. TNF-α binds to two different receptors, which initiate signal transduction pathways. These pathways lead to various cellular responses, including cell survival, differentiation, and proliferation. However, the inappropriate or excessive activation of TNF-α signaling is associated with chronic inflammation and can eventually lead to the development of pathological complications such as autoimmune diseases. The human TNFα gene is located on chromosome 6 (Gene ID: 7124; location NC_000006 (31575565 . . . 31578336). An example of an amino acid sequence for human TNFα is available as UNIPROT accession no. P01375 and shown below as SE ID NO: 11.

MSTESMIRDVELAEEALPKKTGGPQGSRRCLFLSLFSFLIVAGATTLFC
LLHFGVIGPQREEFPRDLSLISPLAQAVRSSSRTPSDKPVAHVVANPQA
EGQLQWLNRRANALLANGVELRDNQLVVPSEGLYLIYSQVLFKGQGCPS
THVLLTHTISRIAVSYQTKVNLLSAIKSPCQRETPEGAEAKPWYEPIYL
GGVFQLEKGDRLSAEINRPDYLDFAESGQVYFGIIAL

A cDNA sequence encoding the SEQ ID NO: 11 TNFα protein is available as NCBI accession no. NM_000594, shown below as SEQ ID NO:12.

| | |
|---|---|
| 1 | AGCAGACGCT CCCTCAGCAA GGACAGCAGA GGACCAGCTA AGAGGGAGAG AAGCAACTAC |
| 61 | AGACCCCCCC TGAAAACAAC CCTCAGACGC CACATCCCCT GACAAGCTGC CAGGCAGGTT |
| 121 | CTCTTCCTCT CACATACTGA CCCACGGCTC CACCCTCTCT CCCCTGGAAA GGACACCATG |
| 181 | AGCACTGAAA GCATGATCCG GGACGTGGAG CTGGCCGAGG AGGCGCTCCC CAAGAAGACA |
| 241 | GGGGGGCCCC AGGGCTCCAG GCGGTGCTTG TTCCTCAGCC TCTTCTCCTT CCTGATCGTG |
| 301 | GCAGGCGCCA CCACGCTCTT CTGCCTGCTG CACTTTGGAG TGATCGGCCC CCAGAGGGAA |
| 361 | GAGTTCCCCA GGGACCTCTC TCTAATCAGC CCTCTGGCCC AGGCAGTCAG ATCATCTTCT |
| 421 | CGAACCCCGA GTGACAAGCC TGTAGCCCAT GTTGTAGCAA ACCCTCAAGC TGAGGGGCAG |
| 481 | CTCCAGTGGC TGAACCGCCG GGCCAATGCC CTCCTGGCCA ATGGCGTGGA GCTGAGAGAT |
| 541 | AACCAGCTGG TGGTGCCATC AGAGGGCCTG TACCTCATCT ACTCCCAGGT CCTCTTCAAG |
| 601 | GGCCAAGGCT GCCCCTCCAC CCATGTGCTC CTCACCCACA CCATCAGCCG CATCGCCGTC |
| 661 | TCCTACCAGA CCAAGGTCAA CCTCCTCTCT GCCATCAAGA GCCCCTGCCA GAGGGAGACC |
| 721 | CCAGAGGGGG CTGAGGCCAA GCCCTGGTAT GAGCCCATCT ATCTGGGAGG GGTCTTCCAG |
| 781 | CTGGAGAAGG GTGACCGACT CAGCGCTGAG ATCAATCGGC CCGACTATCT CGACTTTGCC |
| 841 | GAGTCTGGGC AGGTCTACTT TGGGATCATT GCCCTGTGAG GAGGACGAAC ATCCAACCTT |
| 901 | CCCAAACGCC TCCCCTGCCC CAATCCCTTT ATTACCCCCT CCTTCAGACA CCCTCAACCT |
| 961 | CTTCTGGCTC AAAAAGAGAA TTGGGGGCTT AGGGTCGGAA CCCAAGCTTA GAACTTTAAG |
| 1021 | CAACAAGACC ACCACTTCGA AACCTGGGAT TCAGGAATGT GTGGCCTGCA CAGTGAAGTG |
| 1081 | CTGGCAACCA CTAAGAATTC AAACTGGGGC CTCCAGAACT CACTGGGGCC TACAGCTTTG |
| 1141 | ATCCCTGACA TCTGGAATCT GGAGACCAGG GAGCCTTTGG TTCTGGCCAG AATGCTGCAG |
| 1201 | GACTTGAGAA GACCTCACCT AGAAATTGAC ACAAGTGGAC CTTAGGCCTT CCTCTCTCCA |
| 1261 | GATGTTTCCA GACTTCCTTG AGACACGGAG CCCAGCCCTC CCCATGGAGC CAGCTCCCTC |
| 1321 | TATTTATGTT TGCACTTGTG ATTATTTATT ATTTATTTAT TATTTATTTA TTTACAGATG |
| 1381 | AATGTATTTA TTTGGGAGAC CGGGGTATCC TGGGGGACCC AATGTAGGAG CTGCCTTGGC |
| 1441 | TCAGACATGT TTTCCGTGAA AACGGAGCTG AACAATAGGC TGTTCCCATG TAGCCCCCTG |
| 1501 | GCCTCTGTGC CTTCTTTTGA TTATGTTTTT TAAAATATTT ATCTGATTAA GTTGTCTAAA |
| 1561 | CAATGCTGAT TTGGTGACCA ACTGTCACTC ATTGCTGAGC CTCTGCTCCC CAGGGGAGTT |
| 1621 | GTGTCTGTAA TCGCCCTACT ATTCAGTGGC GAGAAATAAA GTTTGCTTAG |

A Disintegrin and Metalloproteinase with Thrombospondin Motifs 4 (ADAMTS4) ADAMTS4, also known as aggrecanase-1, is expressed by an array of tissues, most prominently in endocrine organs, lungs, and brain, but also in the cardiovascular system. Notably, the majority of ADAMTS4 substrates are principal proteoglycans expressed physiologically in smooth muscle cells (SMCs) of blood vessels and the developing heart. The ADAMTS4 gene is located on chromosome 1 (Gene ID: 9507; location NC_000001 (161184302 . . . 161199054). An example of an amino acid sequence for human ADAMTS-4 is available as UNIPROT accession no. 075173 and shown below as SEQ ID NO: 13.

MSQTGSHPGRGLAGRWLWGAQPCLLLPIVPLSWLVWLLLLLLASLLPSARLASPLPRE
EEIVFPEKLNGSVLPGSGAPARLLCRLQAFGETLLLELEQDSGVQVEGLTVQYLGQAP
ELLGGAEPGTYLTGTINGDPESVASLHWDGGALLGVLQYRGAELHLQPLEGGTPNSAG
GPGAHILRRKSPASGQGPMCNVKAPLGSPSPRPRRAKRFASLSRFVETLVVADDKMAA
FHGAGLKRYLLTVMAAAAKAFKHPSIRNPVSLVVTRLVILGSGEEGPQVGPSAAQTLR
SFCAWQRGLNTPEDSDPDHFDTAILFTRQDLCGVSTCDTLGMADVGTVCDPARSCAIV
EDDGLQSAFTAAHELGHVENMLHDNSKPCISLNGPLSTSRHVMAPVMAHVDPEEPWSP
CSARFITDFLDNGYGHCLLDKPEAPLHLPVTFPGKDYDADRQCQLTFGPDSRHCPQLP
PPCAALWCSGHLNGHAMCQTKHSPWADGTPCGPAQACMGGRCLHMDQLQDENIPQAGG
WGPWGPWGDCSRTCGGGVQFSSRDCTRPVPRNGGKYCEGRRTRFRSCNTEDCPTGSAL
TFREEQCAAYNHRTDLFKSFPGPMDWVPRYTGVAPQDQCKLTCQAQALGYYYVLEPRV
VDGTPCSPDSSSVCVQGRCIHAGCDRIIGSKKKFDKCMVCGGDGSGCSKQSGSFRKER
YGYNNVVTIPAGATHILVRQQGNPGHRSIYLALKLPDGSYALNGEYTLMPSPTDVVLP
GAVSLRYSGATAASETLSGHGPLAQPLTLQVLVAGNPQDTRLRYSFFVPRPTPSTPRP
TPQDWLHRRAQILEILRRRPWAGRK

A cDNA sequence encoding the SEQ ID NO: 13 ADAMTS4 protein is available as NCBI accession no. NM_005099, shown below as SEQ ID NO:14.

| | |
|---|---|
| 1 | GGGAGAACCC ACAGGGAGAC CCACAGACAC ATATGCACGA GAGAGACAGA GGAGGAAAGA |
| 61 | GACAGAGACA AAGGCACAGC GGAAGAAGGC AGAGACAGGG CAGGCACAGA AGCGGCCCAG |
| 121 | ACAGAGTCCT ACAGAGGGAG AGGCCAGAGA AGCTGCAGAA GACACAGGCA GGGAGAGACA |
| 181 | AAGATCCAGG AAAGGAGGGC TCAGGAGGAG AGTTTGGAGA AGCCAGACCC CTGGGCACCT |
| 241 | CTCCCAAGCC CAAGGACTAA GTTTTCTCCA TTTCCTTTAA CGGTCCTCAG CCCTTCTGAA |
| 301 | AACTTTGCCT CTGACCTTGG CAGGAGTCCA AGCCCCCAGG CTACAGAGAG GAGCTTTCCA |
| 361 | AAGCTAGGGT GTGGAGGACT TGGTGCCCTA GACGGCCTCA GTCCCTCCCA GCTGCAGTAC |
| 421 | CAGTGCCATG TCCCAGACAG GCTCGCATCC CGGGAGGGGC TTGGCAGGGC GCTGGCTGTG |
| 481 | GGGAGCCCAA CCCTGCCTCC TGCTCCCCAT TGTGCCGCTC TCCTGGCTGG TGTGGCTGCT |
| 541 | TCTGCTACTG CTGGCCTCTC TCCTGCCCTC AGCCCGGCTG GCCAGCCCCC TCCCCCGGGA |
| 601 | GGAGGAGATC GTGTTTCCAG AGAAGCTCAA CGGCAGCGTC CTGCCTGGCT CGGGCGCCCC |
| 661 | TGCCAGGCTG TTGTGCCGCT TGCAGGCCTT TGGGGAGACG CTGCTACTAG AGCTGGAGCA |
| 721 | GGACTCCGGT GTGCAGGTCG AGGGGCTGAC AGTGCAGTAC CTGGGCCAGG CGCCTGAGCT |
| 781 | GCTGGGTGGA GCAGAGCCTG GCACCTACCT GACTGGCACC ATCAATGGAG ATCCGGAGTC |
| 841 | GGTGGCATCT CTGCACTGGG ATGGGGGAGC CCTGTTAGGC GTGTTACAAT ATCGGGGGGC |
| 901 | TGAACTCCAC CTCCAGCCCC TGGAGGGAGG CACCCCTAAC TCTGCTGGGG GACCTGGGGC |
| 961 | TCACATCCTA CGCCGGAAGA GTCCTGCCAG CGGTCAAGGT CCCATGTGCA ACGTCAAGGC |
| 1021 | TCCTCTTGGA AGCCCCAGCC CCAGACCCCG AAGAGCCAAG CGCTTTGCTT CACTGAGTAG |
| 1081 | ATTTGTGGAG ACACTGGTGG TGGCAGATGA CAAGATGGCC GCATTCCACG GTGCGGGGCT |
| 1141 | AAAGCGCTAC CTGCTAACAG TGATGGCAGC AGCAGCCAAG GCCTTCAAGC ACCCAAGCAT |
| 1201 | CCGCAATCCT GTCAGCTTGG TGGTGACTCG GCTAGTGATC CTGGGGTCAG GCGAGGAGGG |
| 1261 | GCCCCAAGTG GGGCCCAGTG CTGCCCAGAC CCTGCGCAGC TTCTGTGCCT GGCAGCGGGG |
| 1321 | CCTCAACACC CCTGAGGACT CGGACCCTGA CCACTTTGAC ACAGCCATTC TGTTTACCCG |
| 1381 | TCAGGACCTG TGTGGAGTCT CCACTTGCGA CACGCTGGGT ATGGCTGATG TGGGCACCGT |
| 1441 | CTGTGACCCG GCTCGGAGCT GTGCCATTGT GGAGGATGAT GGGCTCCAGT CAGCCTTCAC |
| 1501 | TGCTGCTCAT GAACTGGGTC ATGTCTTCAA CATGCTCCAT GACAACTCCA AGCCATGCAT |
| 1561 | CAGTTTGAAT GGGCCTTTGA GCACCTCTCG CCATGTCATG GCCCCTGTGA TGGCTCATGT |
| 1621 | GGATCCTGAG GAGCCCTGGT CCCCCTGCAG TGCCCGCTTC ATCACTGACT TCCTGGACAA |
| 1681 | TGGCTATGGG CACTGTCTCT TAGACAAACC AGAGGCTCCA TTGCATCTGC CTGTGACTTT |
| 1741 | CCCTGGCAAG GACTATGATG CTGACCGCCA GTGCCAGCTG ACCTTCGGGC CCGACTCACG |
| 1801 | CCATTGTCCA CAGCTGCCGC CGCCCTGTGC TGCCCTCTGG TGCTCTGGCC ACCTCAATGG |
| 1861 | CCATGCCATG TGCCAGACCA AACACTCGCC CTGGGCCGAT GGCACACCCT GCGGGCCCGC |
| 1921 | ACAGGCCTGC ATGGGTGGTC GCTGCCTCCA CATGGACCAG CTCCAGGACT TCAATATTCC |
| 1981 | ACAGGCTGGT GGCTGGGGTC CTTGGGGACC ATGGGGTGAC TGCTCTCGGA CCTGTGGGGG |
| 2041 | TGGTGTCCAG TTCTCCTCCC GAGACTGCAC GAGGCCTGTC CCCCGGAATG GTGGCAAGTA |
| 2101 | CTGTGAGGGC CGCCGTACCC GCTTCCGCTC CTGCAACACT GAGGACTGCC CAACTGGCTC |
| 2161 | AGCCCTGACC TTCCGCGAGG AGCAGTGTGC TGCCTACAAC CACCGCACCG ACCTCTTCAA |
| 2221 | GAGCTTCCCA GGGCCCATGG ACTGGGTTCC TCGCTACACA GGCGTGGCCC CCCAGGACCA |
| 2281 | GTGCAAACTC ACCTGCCAGG CCCAGGCACT GGGCTACTAC TATGTGCTGG AGCCACGGGT |
| 2341 | GGTAGATGGG ACCCCCTGTT CCCCGGACAG CTCCTCGGTC TGTGTCCAGG GCCGATGCAT |
| 2401 | CCATGCTGGC TGTGATCGCA TCATTGGCTC CAAGAAGAAG TTTGACAAGT GCATGGTGTG |
| 2461 | CGGAGGGGAC GGTTCTGGTT GCAGCAAGCA GTCAGGCTCC TTCAGGAAAT TCAGGTACGG |
| 2521 | ATACAACAAT GTGGTCACTA TCCCCGCGGG GGCCACCCAC ATTCTTGTCC GGCAGCAGGG |
| 2581 | AAACCCTGGC CACCGGAGCA TCTACTTGGC CCTGAAGCTG CCAGATGGCT CCTATGCCCT |
| 2641 | CAATGGTGAA TACACGCTGA TGCCCTCCCC CACAGATGTG GTACTGCCTG GGGCAGTCAG |
| 2701 | CTTGCGCTAC AGCGGGGCCA CTGCAGCCTC AGAGACACTG TCAGGCCATG GGCCACTGGC |
| 2761 | CCAGCCTTTG ACACTGCAAG TCCTAGTGGC TGGCAACCCC CAGGACACAC GCCTCCGATA |
| 2821 | CAGCTTCTTC GTGCCCCGGC CGACCCCTTC AACGCCACGC CCCACTCCCC AGGACTGGCT |
| 2881 | GCACCGAAGA GCACAGATTC TGGAGATCCT TCGGCGGCGC CCCTGGGCGG GCAGGAAATA |
| 2941 | ACCTCACTAT CCCGGCTGCC CTTTCTGGGC ACCGGGGCCT CGGACTTAGC TGGGAGAAAG |
| 3001 | AGAGAGCTTC TGTTGCTGCC TCATGCTAAG ACTCAGTGGG GAGGGGCTGT GGGCGTGAGA |
| 3061 | CCTGCCCCTC CTCTCTGCCC TAATGCGCAG GCTGGCCCTG CCCTGGTTTC CTGCCCTGGG |
| 3121 | AGGCAGTGAT GGGTTAGTGG ATGGAAGGGG CTGACAGACA GCCCTCCATC TAAACTGCCC |
| 3181 | CCTCTGCCCT GCGGGTCACA GGAGGGAGGG GGAAGGCAGG GAGGGCCTGG GCCCCAGTTG |
| 3241 | TATTTATTTA GTATTTATTC ACTTTTATTT AGCACCAGGG AAGGGGACAA GGACTAGGGT |
| 3301 | CCTGGGGAAC CTGACCCCTG ACCCCTCATA GCCCTCACCC TGGGGCTAGG AAATCCAGGG |
| 3361 | TGGTGGTGAT AGGTATAAGT GGTGTGTGTA TGCGTGTGTG TGTGTGTGAA AATGTGTGTG |
| 3421 | TGCTTATGTA TGAGGTACAA CCTGTTCTGC TTTCCTCTTC CTGAATTTTA TTTTTTGGGA |
| 3481 | AAAGAAAAGT CAAGGGTAGG GTGGGCCTTC AGGGAGTGAG GGATTATCTT TTTTTTTTTT |
| 3541 | TCTTTCTTTC TTTCTTTTTT TTTTTTGAGA CAGAATCTCG CTCTGTCGCC CAGGCTGGAG |
| 3601 | TGCAATGGCA CAATCTCGGC TCACTGCATC CTCCGCCTCC CGGGTTCAAG TGATTCTCAT |
| 3661 | GCCTCAGCCT CCTGAGTAGC TGGGATTACA GGCTCCTGCC ACCACGCCCG GCTAATTTTT |
| 3721 | GTTTTGTTTT GTTTGGAGAC AGAGTCTCGC TATTGTCACC AGGGCTGGAA TGATTTCAGC |
| 3781 | TCACTGCAAC CTTCGCCACC TGGGTTCCAG CAATTCTCCT GCCTCAGCCT CCCGAGTAGC |
| 3841 | TGAGATTATA GGCACCTACC ACCACGCCCG GCTAATTTTT GTATTTTTAG TAGAGACGGG |
| 3901 | GTTTCACCAT GTTGGCCAGG CTGGTCTCGA ACTCCTGACC TTAGGTGATC CACTCGCCTT |
| 3961 | CATCTCCCAA AGTGCTGGGA TTACAGGCGT GAGCCACCGT GCCTGGCCAC GCCCAACTAA |
| 4021 | TTTTTGTATT TTTAGTAGAG ACAGGGTTTC ACCATGTTGG CCAGGCTGCT CTTGAACTCC |
| 4081 | TGACCTCAGG TAATCGACCT GCCTCGGCCT CCCAAAGTGC TGGGATTACA GGTGTGAGCC |
| 4141 | ACCACGCCCG GTACATATTT TTTAAATTGA ATTCTACTAT TTATGTGATC CTTTTGGAGT |
| 4201 | CAGACAGATG TGGTTGCATC CTAACTCCAT GTCTCTGAGC ATTAGATTTC TCATTTGCCA |
| 4261 | ATAATAATAC CTCCCTTAGA AGTTTGTTGT GAGGATTAAA TAATGTAAAT AAAGAACTAG |
| 4321 | CATAACACTC AGCATCTAGT AAGTGCTCAA CAAATAGCAG CTGCTGTTAC TTACTGTTAT |
| 4381 | CAAATTTCTG TCCACATCCA CTCTCCATAT GCACTTGAAG GTGGCAAAGA TCCACAACCA |
| 4441 | TGGTGCCTGC CTTTATCCTC AGGGTCCGTT CCTTTGGTTG GCAGACCCCT ATCCTGGGTT |
| 4501 | CTGAGGGACC AACAGAGAAA GGAAAATTCC ATCCCTCACC TCTGGAAGTT CCCAATCACA |
| 4561 | GGAAGGAAAC ATAGTAAGCA CGTGGCTACA AATACAATTG ACAAGAACAT GAAGGTGCAG |
| 4621 | GATAACAAGA ACAAATAACA AGAACAACTG CATCAACACA AATGAGTGCT TAGTAATAAG |
| 4681 | GGTGATAGTT GAGGGGTCTG GGTTTCACAA CAGTAGAAAG AGCACTGGAG TGGGAGCCAG |
| 4741 | CGGGTCTGGA TTCAATTTGG GGCTCGGCGT CTTATTAGCT GGGTGGTGTT GGGTAAGTCA |
| 4801 | CTGATGCTGA GCCTTAGATT GCTCATATGG GACTAACAGT ATCTACTCCC ACAGAGTTGT |
| 4861 | TCTGGGAACA AATGCTAGAA TATTTTCAAA ATAGTAAAGG TTATAGTCAT GGCCATGTGA |
| 4921 | GAGGTTACCC CTATGACTAC CTGAAGATGG AACGGAGTCT CCAGAATCTG CCAGTGTAAA |
| 4981 | CCCAGCAGAA TGCCTAGAAG ATGTGAGATT AGAATAAAAT TTCATAAAAC AAAAACAATC |
| 5041 | GGGCACGGTG GCTCATGCCT GTAATCCCAG CACTTTGGGA GGCCGAAATG GGCGGATCAC |
| 5101 | GAGGTCAGGA GATTGAGACC GACCATCCTG GCTACCACAG TGAAACCCCG TGTCTACTAA |
| 5161 | AAATACAAAA TATATATATA TATATATATA TATATTAGCC GGGCATGGTG ACAGGTGCCT |
| 5221 | ATAGTCCCAG CTACTTGGGA GACTGAGGCA GGAGAATGAC TTGCACCCGG GAGGCAGAGC |
| 5281 | TTACAGTGAG TCGAGATTGC GCCACTGCAC TCCAGCCTGG GAGACAGAGT GAGACTCGTC |
| 5341 | TCGATACAAA AACAAAAACA AAAACAGGAT ATGGTTTGGC AGGAAATAGG CAAGAAGGCA |
| 5401 | AAAAGAATAA CCTGGAAAAG GATCTGAGGT AAGGGAGCCA GGTGCCTCAA AATGGCAGAA |
| 5461 | TACCTGATGC CTGAGGAGAG GGAGGGAATA AAACATCTGC ATTTTCCCCT CTGGGCAAGG |
| 5521 | CGCCTTTGCT TGAGAAAGAA TTTTGGGCTG GATGAGATGG TTCACACCTG TAATCCCAGT |
| 5581 | ACTTTGGCAG GAGGATTGCT TGAGGCCAGG AGGTTGGGAC CAACCTGGGC AACATAGAAA |
| 5641 | GATTCCATCT CTACCAAAAA AAAAAAAGAT TGAAAAATTA GCCGGGCGTG GTGGCACCTG |
| 5701 | TAGTCCCAGC TACTTGGGAG GCTAAAATAG GAGGACTGCT TGAGCCCACG ATTTCGAGGC |
| 5761 | TATGGTGAGC TACAATCATG CCACTATACT CCAGCCTGGG TGAGAGACCA AAACACCAAC |
| 5821 | TCAAAAAAAA AAAAAAAAAA AAAAAAAGGG CCAGGCATGG TGGCTCACAC CTGTAATCTC |
| 5881 | AGCACTTTGG GAGGCTGAGG TTCAAAACCA GCCAGGCCAA CATGGTGAAA CCCCATCTCT |
| 5941 | ACTAAAAAAA AAATACAAAA AATTAGCCAG GTGTGGTGGC AAGCGCCTAT AGTCCCAGCT |
| 6001 | ACTCATGAGG CTGAGGCAGG AGGATCGCTT GAACCTGGGA GGTGGAGGTT GCAGTGAGCC |
| 6061 | GAAATTGCGC CATTGCACTC CAGTCTGGGC GATAGAGTGA GACTCCATCT CAAAAAAAAA |
| 6121 | AAAAAAAAAA AAAAGAACTT GGGCAGTCCT CTATGTGTCA TGGATGGAAC AGGGATGGGC |
| 6181 | AAGGGTGGTA GGTAGACCCT GCAAGAATTT GGAGTTTTGA GAGGCAGACG CAGGACTGCT |
| 6241 | AGGGATTGGG GAAGGTACTG GATGTGGGGT TGTGGGAGAA ACTATAGGTA AAGAAGACCC |
| 6301 | TGAGGTTGAG GTGGAAGAGT GAAGAATGGG GGAACCAAAG GCACTTCACT CTGCCATAGC |
| 6361 | AGCCCCTAGC TGGGATGCCA AATACTGCTT GGAATGTGAA GCTGGGACTA TGGGGTTGAG |
| 6421 | GCAGGCCATG GAGGTTGTAG TGGTTATATG TGCTATGCTT ACTGGATCTG GGCTTTGCTA |
| 6481 | ATCAAGTTCT GTACCAGGCA GTGCCTTATA CAGCACCCTG TACTCTACAC CAGCCAGCAC |
| 6541 | AGCGCCTGCT TCCTCTGCAG CAATGAGAAA AGACTGCCCA CACTCTCTTC TCTCCAGTAA |
| 6601 | ACACGGTTCT CCCTGCTAGG CTTGGCCCCC TGGCCCTTCC TGTGGTCCTC CTACTAACCA |
| 6661 | GGCTGAAGAA GATGGAGACA AGACAATAGT GATCTTTACT CGTTTCATCT AGTTTGCAAA |
| 6721 | ATGAGACCAC AGATAGTATG TTTATGGACC TTGATACTAT GAGTTGATGG TACAATGAGC |
| 6781 | AGAGTTCTGT GATAAGTAGA TGTAAGGTAC AAATACAAAC AATATATACG ACTGTCCTAA |
| 6841 | AGGGGTTCAC ACTGCATTCT GGGCAGGGGA GGTTGTATAC TGCTCCCACT CCACCTTCCC |
| 6901 | CGCAAACAGA AGCAATGGGA CAGTAAAAGG GACTAGAAGA TAAGAGCCTA ATGAGCCCTG |
| 6961 | AACTGAATGG ACTATGAGGG TTATGGAGGG CTACCTTGGG CTGGGCCCTG GAGAACAGGT |
| 7021 | CATCATGATA GCCCTTCTCA CTTTCCTTTA ATGTTCATGG AGATACACTT TTTTTTTTGA |
| 7081 | GATAGGGTCT CCCTTTGTTG ACCAGGCAGG AGTACAGTGG TGTGATCTTG GCTCACTGCA |
| 7141 | GCCTTGAACT CTCAGGCTCA AGCAACCCTC CCACCTCAAC CTCCTGAGTA CCTAGGACTA |
| 7201 | CAGGCACGAG CTACCACACC CAGCTAATTT ACTTTTTTTT TTGGTTGAGA CGAGGTCTTA |
| 7261 | TCATATTGCC CAGGCTGGTC TTGAACTCCT GGCCTCAAGG GATCTTCCCA CCTCGATCTC |
| 7321 | CCAAAGTGTT GGGATGACCA GTGTGAACCA CCATGCCTGG CCTTTTCTTT TGAGACAGTC |
| 7381 | TCGCTCTGTG GCTCAGGTTG GAGTGCAGTG GCACCATCAT AACTTACTAC AGCCTTGAAC |
| 7441 | TCCTGGGCTC GAGTAATCCT CCCACCTCAG CCTCCTAAGT AGCTTGGACT ACAGGTGTAG |
| 7501 | ATTCACTTTT AAATCACTTT TCTGCCTGTT ATTTTAAGAG AAAGCCCTTC GCTCACCAAG |
| 7561 | TAGGGTTCTC ACTGGCATAA GTAAGTTTTT CTGAAAATGA GACTGAGCTT AGGGGCCACC |
| 7621 | TCTGTGATTG GGTAAGTAAG CAGGAGCTAG AGTGAAAGAG GGGCAGAGAG AGCTAGCTCA |
| 7681 | TCGAAGAGAA GTGATGGGCA TCTCAGCCCT CAATCCCAGG CAAGGCCTGA CCCTTTTCTG |
| 7741 | GAATGGTCTC TGCCTTACCA CACCTCCAAG ACCCAGATTG TAGGTAGGCT TTACCTAATA |
| 7801 | GGAGTCCAAC AAGTTCTTGC TAGGGATGGG ATTGTGGGGG GGCACATATC TCCCTGTAAG |
| 7861 | AGCCACAGTG AGCTCTGTTT CCCATCTGCC TACTTATTGC CCTCGTGGGC CTGAGCTGGG |
| 7921 | GTGTAGATAT ATAAGGAGGA ACAGAAACAG TAATAACAGG AAGTAAGGAG GCACTGAAAC |
| 7981 | AATTCAAGTA TCTGGTCCTG GAGATGAAAA GAGGAAATGG AAAAAGAAAA GGCATTGAAT |
| 8041 | GACAAGGGAT TAGAACCCTG TCCCTAGAGA AGTTCAAGAA GAGTTTGGAC AACCATTTTA |
| 8101 | TGAGATGGCA AGTGTTGTGC ATGAGTATGT GTTTGAGGTG GGGGAGGAAG TTGAAGCAAT |
| 8161 | AAGTTGGGCC AATTTCCTAA AGTCCCTTCT GCCCCTGAGA TTCTCTGCTT CTGTGGTTCA |
| 8221 | AACAGAGGAA ATCTGGACAT TTGTCCAGCC ACTATTTTTG TAATGGAGGT GGGACAGCCA |
| 8281 | GGGTTCCTGG AGTGTGCTGT GGAATCGGCA TTTACACTTC TCTTCCATCT CTAGTCTCAT |
| 8341 | TTCTGTAATC TTCATACAGT ATTACAGTAT TTTGTTTTCT AAAATACCAG ATTGGCAGTA |
| 8401 | AGTGACAAAA CTAGCATCTC ATTGGTCAGG GCTTGTGGGG GAGGAAGGGA GAAGAAAGTG |
| 8461 | GATTTCAATG TATTAACATT TTATTGGCTG AGAGGATGTT TCTGAACCAA TTGAGTACCT |
| 8521 | TCCCCATTGA CTGTTGCTGG GGCCAAGCCA AAATCATTGG CACCACGAGA AACCAGATGA |
| 8581 | CAGCTGGAGG AAACCACGAG TGCTAAAAAT GCTCAGAAAG AGGGGGGTCA GGGAAGGCGG |
| 8641 | GGGAGCAACA ACACTGAACA ACTTCCCCAG GCAGGATCTT ACATGGGGAC AGACCCAGGG |
| 8701 | TACTTAAGTA GCATTAGGAA GGAAAGGGAG GGGAGGAAGT AGATTAAGTA TCCCTCACAG |
| 8761 | TCTTGGCACA ACAAACAGGC ACCATACACC CACACCAACA GTGACACATT GACATAACAC |
| 8821 | ACATCCAACT TCACAGAGAC ACACTAGCAC ATTCTCTCTT TTTTTTAAAA TTTATTTTAT |
| 8881 | TATTATTATA CTTTAAGTTT TAGGGTACAT GTGCACAATG TGCAGGTTAG TTACATATGT |
| 8941 | ATACATGTGC CATGCTGGTG TGCTGCACCC ATTAACTCAT CATTTAGCAT TAGGTATATC |
| 9001 | TCCTAATGCT ATCCCTCCCC CCTCCCCCCC TGCCTTTCTT TTTCTTCTTT TTTTTCTTAA |
| 9061 | GACAGATTCT CATTCTGTCA CCCAGGCTAG AGTGCAATGG CGTGATCTCG GCTCACTGCA |
| 9121 | ACCTCCACCT CCCAGGTTCA AGTGATTCTC CTGCCTCAGC CTGCCAAGTA GCTGGGATTA |
| 9181 | CAGGTATGCA CCACCATGCC CAGCTAATTT TTTGTATTTT TAGTATAGAG ACGGGGTTTT |
| 9241 | CCATGTTGCC CAGGCTGGTC TTGAACTCCC GGGCTCAAGT GATCTGCCTG CCTCGGCCTC |
| 9301 | CCAAAGTGCT GGGATTACAG GTGTGAGCCA GGGTGCCTGG CCAACACATT CTGTTGATAA |
| 9361 | TATAGATGCA AATAAGTAGT AGGGAGCGGA AAATGTAAGA ATTTTCACCT GAAAATGGAT |
| 9421 | CTGAAGAGTA AAAGATATTA ATTGACCTAA TCGTGCATTG AGAATTGACT GTTTATCCCA |
| 9481 | TTTATTTGTT CAATAAATAA GCCAATGGGT TGTAAACATT GTAAACATTC CACAGCATGT |
| 9541 | GGAAAGGACA TGGATTTTTG GAGTCAAATT GAATCTAAGC CTTGCTACTT ACTTACCTGT |
| 9601 | AATGATGCAA CCTTAATTAC TTGATCTCTG TAAACCTCAG TTTCCTTACC TACAAAACGT |
| 9661 | TTGTACTATT ACCTGCCTTA TAGAGAGGTC CAGACTTAAG AATATGATGA TGTAAATGTT |
| 9721 | TGGCACTGTG TCTGGCTTTT AGTAAGTGGT CAATAAAGGC CAGTTCTCCT TTCCTTA |

### P-Selectin (SELP)

SELP, also known as aggrecanase-1, is expressed by an array of tissues, most prominently in endocrine organs, lungs, and brain, but also in the cardiovascular system. Notably, the majority of SELP substrates are principal proteoglycans expressed physiologically in smooth muscle cells (SMCs) of blood vessels and the developing heart. The human SELP gene is located on chromosome 1 (Gene ID: 6403; location: NC_000001 (169588849 . . . 169630124). An example of an amino acid sequence for SELP is available as UNIPROT accession no. P16109 and shown below as SEQ ID NO: 15.

MANCQIAILYQRFQRVVFGISQLLCFSALISELTNQKEVAAWTYHYSTKAYSWNISRK
YCQNRYTDLVAIQNKNEIDYLNKVLPYYSSYYWIGIRKNNKTWTWVGTKKALTNEAEN
WADNEPNNKRNNEDCVEIYIKSPSAPGKWNDEHCLKKKHALCYTASCQDMSCSKQGEC
LETIGNYTCSCYPGFYGPECEYVRECGELELPQHVLMNCSHPLGNFSENSQCSFHCTD
GYQVNGPSKLECLASGIWINKPPQCLAAQCPPLKIPERGNMTCLHSAKAFQHQSSCSF
SCEEGFALVGPEVVQCTASGVWTAPAPVCKAVQCQHLEAPSEGTMDCVHPLTAFAYGS
SCKFECQPGYRVRGLDMLRCIDSGHWSAPLPTCEAISCEPLESPVHGSMDCSPSLRAF
QYDTNCSFRCAEGFMLRGADIVRCDNLGQWTAPAPVCQALQCQDLPVPNEARVNCSHP
FGAFRYQSVCSFTCNEGLLLVGASVLQCLATGNWNSVPPECQAIPCTPLLSPQNGTMT
CVQPLGSSSYKSTCQFICDEGYSLSGPERLDCTRSGRWTDSPPMCEAIKCPELFAPEQ
GSLDCSDTRGEFNVGSTCHFSCDNGFKLEGPNNVECTTSGRWSATPPTCKGIASLPTP
GLQCPALTTPGQGTMYCRHHPGTFGFNTTCYFGCNAGFTLIGDSTLSCRPSGQWTAVT
PACRAVKCSELHVNKPIAMNCSNLWGNFSYGSICSFHCLEGQLLNGSAQTACQENGHW
STTVPTCQAGPLTIQEALTYFGGAVASTIGLIMGGTLLALLRKRFRQKDDGKCPLNPH
SHLGTYGVFTNAAFDPSP

A cDNA sequence encoding the SEQ ID NO: 16 SELP protein is available as NCBI accession no. NM 003005, shown below as SEQ ID NO:16.

| | |
|---|---|
| 1 | AGCAGTCTGG GTTGGGCAGA AGGCAGAAAA CCAGCAGAGT CACAGAGGAG ATGGCCAACT |
| 61 | GCCAAATAGC CATCTTGTAC CAGAGATTCC AGAGAGTGGT CTTTGGAATT TCCCAACTCC |
| 121 | TTTGCTTCAG TGCCCTGATC TCTGAACTAA CAAACCAGAA AGAAGTGGCA GCATGGACTT |
| 181 | ATCATTACAG CACAAAAGCA TACTCATGGA ATATTTCCCG TAAATACTGC CAGAATCGCT |
| 241 | ACACAGACTT AGTGGCCATC CAGAATAAAA ATGAAATTGA TTACCTCAAT AAGGTCCTAC |
| 301 | CCTACTACAG CTCCTACTAC TGGATTGGGA TCCGAAAGAA CAATAAGACA TGGACATGGG |
| 361 | TGGGAACCAA AAAGGCTCTC ACCAACGAGG CTGAGAACTG GGCTGATAAT GAACCTAACA |
| 421 | ACAAAAGGAA CAACGAGGAC TGCGTGGAGA TATACATCAA GAGTCCGTCA GCCCCTGGCA |
| 481 | AGTGGAATGA TGAGCACTGC TTGAAGAAAA AGCACGCATT GTGTTACACA GCCTCCTGCC |
| 541 | AGGACATGTC CTGCAGCAAA CAAGGAGAGT GCCTCGAGAC CATCGGGAAC TACACCTGCT |
| 601 | CCTGTTACCC TGGATTCTAT GGGCCAGAAT GTGAATACGT GAGAGAGTGT GGAGAACTTG |
| 661 | AGCTCCCTCA ACACGTGCTC ATGAACTGCA GCCACCCTCT GGGAAACTTC TCTTTTAACT |
| 721 | CGCAGTGCAG CTTCCACTGC ACTGACGGGT ACCAAGTAAA TGGGCCCAGC AAGCTGGAAT |
| 781 | GCTTGGCTTC TGGAATCTGG ACAAATAAGC CTCCACAGTG TTTAGCTGCC CAGTGCCCAC |
| 841 | CCCTGAAGAT TCCTGAACGA GGAAACATGA CCTGCCTTCA TTCTGCAAAA GCATTCCAGC |
| 901 | ATCAGTCTAG CTGCAGCTTC AGTIGTGAAG AGGGATTTGC ATTAGTTGGA CCGGAAGTGG |
| 961 | TGCAATGCAC AGCCTCGGGG GTATGGACAG CCCCAGCCCC AGTGTGTAAA GCTGTGCAGT |
| 1021 | GTCAGCACCT GGAAGCCCCC AGTGAAGGAA CCATGGACTG TGTTCATCCG CTCACTGCTT |
| 1081 | TTGCCTATGG CTCCAGCTGT AAATTTGAGT GCCAGCCCGG CTACAGAGTG AGGGGCTTGG |
| 1141 | ACATGCTCCG CTGCATTGAC TCTGGACACT GGTCTGCACC CTTGCCAACC TGTGAGGCTA |
| 1201 | TTTCGTGTGA GCCGCTGGAG AGTCCTGTCC ACGGAAGCAT GGATTGCTCT CCATCCTTGA |
| 1261 | GAGCGTTTCA GTATGACACC AACTGTAGCT TCCGCTGTGC TGAAGGTTTC ATGCTGAGAG |
| 1321 | GAGCCGATAT AGTTCGGTGT GATAACTTGG GACAGTGGAC AGCACCAGCC CCAGTCTGTC |
| 1381 | AAGCTTTGCA GTGCCAGGAT CTCCCAGTTC CAAATGAGGC CCGGGTGAAC TGCTCCCACC |
| 1441 | CCTTCGGTGC CTTTAGGTAC CAGTCAGTCT GCAGCTTCAC CTGCAATGAA GGCTTGCTCC |
| 1501 | TGGTGGGAGC AAGTGTGCTA CAGTGCTTGG CTACTGGAAA CTGGAATTCT GTTCCTCCAG |
| 1561 | AATGCCAAGC CATTCCCTGC ACACCTTTGC TAAGCCCTCA GAATGGAACA ATGACCTGTG |
| 1621 | TTCAACCTCT TGGAAGTTCC AGTTATAAAT CCACATGTCA ATTCATCTGT GACGAGGGAT |
| 1681 | ATTCTTTGTC TGGACCAGAA AGATTGGATT GTACTCGATC GGGACGCTGG ACAGACTCCC |
| 1741 | CACCAATGTG TGAAGCCATC AAGTGCCCAG AACTCTTTGC CCCAGAGCAG GGCAGCCTGG |
| 1801 | ATTGTTCTGA CACTCGTGGA GAATTCAATG TTGGCTCCAC CTGCCATTTC TCTTGTGACA |
| 1861 | ACGGCTTTAA GCTGGAGGGG CCCAATAATG TGGAATGCAC AACTTCTGGA AGATGGTCAG |
| 1921 | CTACTCCACC AACCTGCAAA GGCATAGCAT CACTTCCTAC TCCAGGGGTG CAATGTCCAG |
| 1981 | CCCTCACCAC TCCTGGGCAG GGAACCATGT ACTGTAGGCA TCATCCGGGA ACCTTTGGTT |
| 2041 | TTAATACCAC TTGTTACTTT GGCTGCAACG CTGGATTCAC ACTCATAGGA GACAGCACTC |
| 2101 | TCAGCTGCAG ACCTTCAGGA CAATGGACAG CAGTAACTCC AGCATGCAGA GCTGTGAAAT |
| 2161 | GCTCAGAACT ACATGTTAAT AAGCCAATAG CGATGAACTG CTCCAACCTC TGGGGAAACT |
| 2221 | TCAGTTATGG ATCAATCTGC TCTTTCCATT GTCTAGAGGG CCAGTTACTT AATGGCTCTG |
| 2281 | CACAAACAGC ATGCCAAGAG AATGGCCACT GGTCAACTAC CGTGCCAACC TGCCAAGCAG |
| 2341 | GACCATTGAC TATCCAGGAA GCCCTGACTT ACTTTGGTGG AGCGGTGGCT TCTACGATAG |
| 2401 | GTCTGATAAT GGGTGGGACG CTCCTGGCTT TGCTAAGAAA GCGTTTCAGA CAAAAAGATG |
| 2461 | ATGGGAAATG CCCCTTGAAT CCTCACAGCC ACCTAGGAAC ATATGGAGTT TTTACAAACG |
| 2521 | CTGCATTTGA CCCGAGTCCT TAAGGTTTCC ATAAACACCC ATGAATCAAA GACATGGAAT |
| 2581 | TACCTTAGAT TAGCTCTGGA CCAGCCTGTT GGACCCGCTC TGGACCAACC CTGTTTCCTG |
| 2641 | AGTTTGGGAT TGTGGTACAA TCTCAAATTC TCAACCTACC ACCCCTTCCT GTCCCACCTC |
| 2701 | TTCTCTTCCT GTAACACAAG CCACAGAAGC CAGGAGCAAA TGTTTCTGCA GTAGTCTCTG |
| 2761 | TGCTTTGACT CACCTGTTAC TTGAAATACC AGTGAACCAA AGAGACTGGA GCATCTGACT |
| 2821 | CACAAGAAGA CCAGACTGTG GAGAAATAAA AATACCTCTT TATTTTTTGA TTGAAGGAAG |
| 2881 | GTTTTCTCCA CTTTGTTGGA AAGCAGGTGG CATCTCTAAT TGGAAGAAAT TCCTGTAGCA |
| 2941 | TCTTCTGGAG TCTCCAGTGG TTGCTGTTGA TGAGGCCTCT TGGACCTCTG CTCTGAGGCT |
| 3001 | TCCAGAGAGT CCTCTGGATG GCACCAGAGG CTGCAGAAGG CCAAGAATCA AGCTAGAAGG |
| 3061 | CCACATGTCA CCGTGGACCT TCCTGCCACC AGTCACTGTC CCTCAAATGA CCCAAAGACC |
| 3121 | AATATTCAAA TGCGTAATTA AAAGAATTTT CCCCAAA |

### Nuclear Receptor Subfamily 4 Group a Member 3 (NR4A3)

NR4A3 is a transcriptional activator that binds to regulatory elements in promoter regions in a cell- and response element (target)-specific manner. Induces gene expression by binding as monomers to the NR4A1 response element (NBRE) 5′-AAAAGGTCA-3′ site and as homodimers to the Nur response element (NurRE) site in the promoter of their regulated target genes. The human NR4A3 gene is located on chromosome 9 (Gene ID: 8013; location: NC_000009 (99821885 . . . 99866891). An example of an amino acid sequence for NR4A3 is available as UNIPROT accession no. Q92570 and shown below as SEQ ID NO: 17.

MPCVQAQYSPSPPGSSYAAQTYSSEYTTEIMNPDYTKLIMDLGSTEITATATTSLPSI
STFVEGYSSNYELKPSCVYQMQRPLIKVEEGRAPSYHHHHHHHHHHHHHHQQQHQQPS
IPPASSPEDEVLPSTSMYFKQSPPSTPTTPAFPPQAGALWDEALPSAPGCIAPGPLLD
PPMKAVPTVAGARFPLFHFKPSPPHPPAPSPAGGHHLGYDPTAAAALSLPLGAAAAAG
SQAAALESHPYGLPLAKRAAPLAFPPLGLTPSPTASSLLGESPSLPSPPSRSSSSGEG
TCAVCGDNAACQHYGVRTCEGCKGFFKRTVQKNAKYVCLANKNCPVDKRRRNRCQYCR
FQKCLSVGMVKEVVRTDSLKGRRGRLPSKPKSPLQQEPSQPSPPSPPICMMNALVRAL
TDSTPRDLDYSRYCPTDQAAAGTDAEHVQQFYNLLTASIDVSRSWAEKIPGFTDLPKE
DQTLLIESAFLELFVLRLSIRSNTAEDKFVFCNGLVLHRLQCLRGFGEWLDSIKDESL
NLQSLNLDIQALACLSALSMITERHGLKEPKRVEELCNKITSSLKDHQSKGQALEPTE
SKVLGALVELRKICTLGLQRIFYLKLEDLVSPPSIIDKLFLDTLPF

A cDNA sequence encoding the SEQ ID NO: 17 NR4A3 protein is available as NCBI accession no. NM_006981, shown below as SEQ ID NO:18.

| | |
|---|---|
| 1 | GCGCAGCCGG GAGAGCGGAG TCTCCTGCCT CCCGCCCCCC ACCCCTCCAG CTCCTGCTCC |
| 61 | TCCTCCGCTC CCCATACACA GACGCGCTCA CACCCGCTCC CTCACTCGCA CACACAGACA |
| 121 | CAAGCGCGCA CACAGGCTCC GCACACACAC TTCGCTCTCC CGCGCGCTCA CACCCCTCTT |
| 181 | GCCCTGAGCC CTTGCCGGTG CAGCGCGGCG CCGCAGCTGG ACGCCCCTCC CGGGCTCACT |
| 241 | TTGCAACGCT GACGGTGCCG GCAGTGGCCG TGGAGGTGGG AACAGCGGCG GCATCCTCCC |
| 301 | CCCTGGTCAC AGCCCAAGCC AGGACGCCCG CGGAACCTCT CGGCTGTGCT CTCCCATGAG |
| 361 | TCGGGATCGC AGCATCCCCC ACCAGCCGCT CACCGCCTCC GGGAGCCGCT GGGCTTGTAC |
| 421 | ACCGCAGCCC TTCCGGGACA GCAGCTGTGA CTCCCCCCCA GTGCAGATTT CGGGACAGCT |
| 481 | CTCTAGAAAC TCGCTCTAAA GACGGAACCG CCACAGCACT CAAAGCCCAC TGCGGAAGAG |
| 541 | GGCAGCCCGG CAAGCCCGGG CCCTGAGCCT GGACCCTTAG CGGTGCCGGG CAGCACTGCC |
| 601 | GGCGCTTCGC CTCGCCGGAC GTCCGCTCCT CCTACACTCT CAGCCTCCGC TGGAGAGACC |
| 661 | CCCAGCCCCA CCATTCAGCG CGCAAGATAC CCTCCAGATA TGCCCTGCGT CCAAGCCCAA |
| 721 | TATAGCCCTT CCCCTCCAGG TTCCAGTTAT GCGGCGCAGA CATACAGCTC GGAATACACC |
| 781 | ACGGAGATCA TGAACCCCGA CTACACCAAG CTGACCATGG ACCTTGGCAG CACTGAGATC |
| 841 | ACGGCTACAG CCACCACGTC CCTGCCCAGC ATCAGTACCT TCGTGGAGGG CTACTCGAGC |
| 901 | AACTACGAAC TCAAGCCTTC CTGCGTGTAC CAAATGCAGC GGCCCTTGAT CAAAGTGGAG |
| 961 | GAGGGGGGGG CGCCCAGCTA CCATCACCAT CACCACCACC ACCACCACCA CCACCACCAT |
| 1021 | CACCAGCAGC AGCATCAGCA GCCATCCATT CCTCCAGCCT CCAGCCCGGA GGACGAGGTG |
| 1081 | CTGCCCAGCA CCTCCATGTA CTTCAAGCAG TCCCCACCGT CCACCCCCAC CACGCCGGCC |
| 1141 | TTCCCCCCGC AGGCGGGGGC GTTATGGGAC GAGGCACTGC CCTCGGCGCC CGGCTGCATC |
| 1201 | GCACCCGGCC CGCTGCTGGA CCCGCCGATG AAGGCGGTCC CCACGGTGGC CGGCGCGCGC |
| 1261 | TTCCCGCTCT TCCACTTCAA GCCCTCGCCG CCGCATCCCC CCGCGCCCAG CCCGGCCGGC |
| 1321 | GGCCACCACC TCGGCTACGA CCCGACGGCC GCTGCCGCGC TCAGCCTGCC GCTGGGAGCC |
| 1381 | GCAGCCGCCG CGGGCAGCCA GGCCGCCGCG CTTGAGAGCC ACCCGTACGG GCTGCCGCTG |
| 1441 | GCCAAGAGGG CGGCCCCGCT GGCCTTCCCG CCTCTCGGCC TCACGCCCTC CCCTACCGCG |
| 1501 | TCCAGCCTGC TGGGCGAGAG TCCCAGCCTG CCGTCGCCGC CCAGCAGGAG CTCGTCGTCT |
| 1561 | GGCGAGGGCA CGTGTGCCGT GTGCGGGGAC AACGCCGCCT GCCAGCACTA CGGCGTGCGA |
| 1621 | ACCTGCGAGG GCTGCAAGGG CTTTTTCAAG AGAACAGTGC AGAAAAATGC AAAATATGTT |
| 1681 | TGCCTGGCAA ATAAAAACTG CCCAGTAGAC AAGAGACGTC GAAACCGATG TCAGTACTGT |
| 1741 | CGATTTCAGA AGTGTCTCAG TGTTGGAATG GTAAAAGAAG TTGTCCGTAC AGATAGTCTG |
| 1801 | AAAGGGAGGA GAGGTCGTCT GCCTTCCAAA CCAAAGAGCC CATTACAACA GGAACCTTCT |
| 1861 | CAGCCCTCTC CACCTTCTCC TCCAATCTGC ATGATGAATG CCCTTGTCCG AGCTTTAACA |
| 1921 | GACTCAACAC CCAGAGATCT TGATTATTCC AGATACTGTC CCACTGACCA GGCTGCTGCA |
| 1981 | GGCACAGATG CTGAGCATGT GCAACAATTC TACAACCTCC TGACAGCCTC CATTGATGTA |
| 2041 | TCCAGAAGCT GGGCAGAAAA GATTCCGGGA TTTACTGATC TCCCCAAAGA AGATCAGACA |
| 2101 | TTACTTATTG AATCAGCCTT TTTGGAGCTG TTTGTCCTCA GACTTTCCAT CAGGTCAAAC |
| 2161 | ACTGCTGAAG ATAAGTTTGT GTTCTGCAAT GGACTTGTCC TGCATCGACT TCAGTGCCTT |
| 2221 | CGTGGATTTG GGGAGTGGCT CGACTCTATT AAAGACTTTT CCTTAAATTT GCAGAGCCTG |
| 2281 | AACCTTGATA TCCAAGCCTT AGCCTGCCTG TCAGCACTGA GCATGATCAC AGAAAGACAT |
| 2341 | GGGTTAAAAG AACCAAAGAG AGTCGAAGAG CTATGCAACA AGATCACAAG CAGTTTAAAA |
| 2401 | GACCACCAGA GTAAGGGACA GGCTCTGGAG CCCACCGAGT CCAAGGTCCT GGGTGCCCTG |
| 2461 | GTAGAACTGA GGAAGATCTG CACCCTGGGC CTCCAGCGCA TCTTCTACCT GAAGCTGGAA |
| 2521 | GACTTGGTGT CTCCACCTTC CATCATTGAC AAGCTCTTCC TGGACACCCT ACCTTTCTAA |
| 2581 | TCAGGAGCAG TGGAGCAGTG AGCTGCCTCC TCTCCTAGCA CCTGCTTGCT ACGCAGCAAA |
| 2641 | GGGATAGGTT TGGAAACCTA TCATTTCCTG TCCTTCCTTA AGAGGAAAAG CAGCTCCTGT |
| 2701 | AGAAAGCAAA GACTTTCTTT TTTTTCTGGC TCTTTTCCTT ACAACCTAAA GCCAGAAAAC |
| 2761 | TTGCAGAGTA TTGTGTTGGG GTTGTGTTTT ATATTTAGGC ATTGGGGGAT GGGGTGGGAG |
| 2821 | GGGGTTATAG TTCATGAGGG TTTTCTAAGA AATTGCTAAC AAAGCACTTT TGGACAATGC |
| 2881 | TATCCCAGCA GGAAAAAAAA GGATAATATA ACTGTTTTAA AACTCTTTCT GGGGAATCCA |
| 2941 | ATTATAGTTG CTTTGTATTT AAAAACAAGA ACAGCCAAGG GTTGTTCGCC AGGGTAGGAT |
| 3001 | GTGTCTTAAA GATTGGTCCC TTGAAAATAT GCTTCCTGTA TCAAAGGTAC GTATGTGGTG |
| 3061 | CAAACAAGGC AGAAACTTCC TTTTAATTTC CTTCTTCCTT TATTTTAACA AATGGTGAAA |
| 3121 | GATGGAGGAT TACCTACAAA TCAGACATGG CAAAACAATA ATGGCTGTTT GCTTCCATAA |
| 3181 | ACAAGTGCAA TTTTTTAAAG TGCTGTCTTA CTAAGTCTTG TTTATTAACT CTCCTTTATT |
| 3241 | CTATATGGAA ATAAAAAGGA GGCAGTCATG TTAGCAAATG ACACGTTAAT ATCCCTAGCA |
| 3301 | GAGGCTGTGT TCACCTTCCC TGTCGATCCC TTCTGAGGTA TGGCCCATCC AAGACTTTTA |
| 3361 | GGCCATTCTT GATGGAACCA GATCCCTGCC CTGACTGTCC AGCTATCCTG AAAGTGGATC |
| 3421 | AGATTATAAA CTGGATTACA TGTAACTGTT TTGGTTGTGT TCTATCAACC CCACCAGAGT |
| 3481 | TCCCTAAACT TGCTTCAGTT ATAGTAACTG ACTGGTATAT TCATTCAGAA GCGCCATAAG |
| 3541 | TCAGTTGAGT ATTTGATCCC TAGATAAGAA CATGCAAATC AGCAGGAACT GGTCATACAG |
| 3601 | GGTAAGCACC AGGGACAATA AGGATTTTTA TAGATATAAT TTAATTTTTG TTATTGGTTA |
| 3661 | AGGAGACAAT TTTGGAGAGC AAGCAAATCT TTTTAAAAAA TAGTATGAAT GTGAATACTA |
| 3721 | GAAAAGATTT AAAAAATAGT ATGAGTGTGA GTACTAGGAA GGATTAGTGG GCTGCGTTTC |
| 3781 | AACATTCCGT GTTCGTACTC CCTTTTGTAT GTTTCTACTG TTAATGCCAT ATTACTATGA |
| 3841 | GATAATTTGT TGCATAGTGT CCTTATTTGT ATAAACATTT GTATGCACGT TATATTGTAA |
| 3901 | TAGCTTTGCC TGTATTTATT GCAAGACCAC CAGCTCCTGG AAGCTGAGTT ACAGAGTAAT |
| 3961 | TAAATGGGGT GTTCACAGTG ACTTGGATAC ACCAATTAGA AATTAAATAA GCAAATATAT |
| 4021 | ATATATATAT AAATATAGCA GGTTACATAT ATATATTTAT AATGTGTCTT TTTATTAACC |
| 4081 | ATTTGTACAA TAAATGTCAC TTCCCATGCC GTTATTTTAT GGTTCATTTG CAGTGACTTT |
| 4141 | TAAGGCAGTA CTGTTTAGCA CTTTGATATT AAAATTTTGC TTATGTTTTG CTAAATTCGA |
| 4201 | ATAATGTTTG AAGATTTTTA GGTCTAAAAG TCTTTATATT ATATACTCTG TATCAAGTCA |
| 4261 | AAATATCTTT GGCCATTTTG CTAAGAAACA AACTTTGAAT GTCAAACTGA TGTCACAGTA |
| 4321 | GTTTTTGTTA GCTTTAAATC ATTTTTGCTT TAGTCTTTTT AAAGGAAAAT AACAAAACTA |
| 4381 | TGCTGTTTAT ATTGTCATTA AATTATACAA TCAAACAAAT GCCAAATGAA TTGCCTAATT |
| 4441 | GCTGCAAAGT ATAACCCAGA TAGGAAATCA TATGTTTTTT TCCAAGAGTC ATTCTAATAT |
| 4501 | TTGATTATGT TATGTGTGCT TTTATGAAAG ATTGTTATTT TTATATATCA AGATGATAGA |
| 4561 | ACCTGGAATG TTAGGATTTT GAAATGTTAG ACTTGGAAGG GGCCTGGTCT GTCAACTAGT |
| 4621 | CCAACCCCTT AAAATTCATA GAGGAGCAAA CTGGGGCCCA TTGAAGGGTG AAGAGTTACT |
| 4681 | CAAGGTCAAA CAGCTGGTAA CAGAATCAAG ACTAAGACCT AATTTACCTT TCCATACTCT |
| 4741 | TTTTTTTTCT CAACTTCATC TATATAAAAT CAGGCTTTTA AACATAACCA CTAATATTTA |
| 4801 | CCTGAAGATA ACCATGAGTA AAGTATACTT TTGCATTAAT TTTTTGAGCT TATATGCAAA |
| 4861 | CATAATAAAT ATTATTAAAT ATCAGGAAAG CTAACATTTC ATACAAGATA GCTTCAGACC |
| 4921 | AAATTCAAAT TGAATTTGAA TAAATTAGAA ATACTGTGCA TACATAACCT TCTTGTGCAC |
| 4981 | CATGAGTATT TGGAAAGTTA ATCCTTGTTT TTGTCGTGTC TATAAAGGAA GAACAAAACA |
| 5041 | AAATAAAAAC AGAGCCCTAG AGAAATGCTG TTACTTTTTA TTTTTACACC CATCAGATTT |
| 5101 | AAGGAAAAGA CTTTTTAGCC ATTATAATCT AGTGGTTGGA AGGAATGAAG AAGCTTTTTT |
| 5161 | AGTAATAGGT CCAGATATGA GTGCTAAAAA TAAAGATGAT AGCATGTTCT TCTGTCTTCC |
| 5221 | ATAGTTATTA CAACTATGAG AGCCTCCCAA GTCATCTTAT CAACTCAACT CCCTTTTTTT |
| 5281 | TGTCTTAATG TTGCACATAA GTTTATACAG AGTGGATGAC CACACTAGCA CAGAAGAGAA |
| 5341 | CAACATGTAT TAAAGCAGGT GATTCCTCCC CTTGGCGGGA GAGCTCTCTC AGTGTGAACA |
| 5401 | TGCCTTCTGT GGGCGGAAAT CAGGAAGCCA CCAGCTGTTA ATGGAGAGTG CCTTGCTTTT |
| 5461 | ATTTCAGACA GCAGAGTTTT CCAAAGTTTC TCTGCTCCTC TAACAGCATT GCTCTTTAGT |
| 5521 | GTGTGTTAAC CTGTGGTTTG AAAGAAATGC TCTTGTACAT TAACAATGTA AATTTAAATG |
| 5581 | ATTAAATTAC ATTTTATCAA TGGC |

In embodiments, the human genes associated with PCa disease progression prediction can include:

Forkhead box P3 (FOXP3): UNIPROT accession no. Q9BZS1; Gene ID: 50943, NCBI accession no. NM_001114377.

Arginase-1 (ARG1): UNIPROT accession no. P05089; Gene ID: 383, NCBI accession no. NM 000045.

### CEBPDP

Proenkephalin-A (PENK): UTNIPROT accession no. P01210; Gene ID: 5179, NCBI accession no. NM_001135690.

Fos-related antigen 1 (FOSL1): UNIPROT accession no. P15407; Gene ID: 8061, NCBI accession no. NM_001300844 XM_005274311.

Dual specificity protein phosphatase 1 (DUSP1): UNIPROT accession no. P28562; Gene ID: 1843, NCBI accession no. NM_004417.

Actin, alpha skeletal muscle (ACTA1): UNIPROT accession no. P68133; Gene ID: 1843, NCBI accession no. NM 001100,

Angiotensinogen (AGT): UNIPROT accession no. P01019; Gene ID: 183.

Cyclic AMP-dependent transcription factor (ATF-3): UNIPROT accession no. P18847; Gene ID: 467, NCBI accession no. NM 001030287.

Cyclin-dependent kinase 1 (CDK1): UNIPROT accession no. P06493; Gene ID: 983, NCBI accession no. NM_001786.

Interleukin 8 (IL-8 or chemokine (C-X-C motif) ligand 8, CXCL8): UNIPROT accession no. P10145; Gene ID: 3576, NCBI accession no. NM 000584.

Versican core protein (VCAN): UNIPROT accession no. P13611; Gene ID: 1462, NCBI accession no. NM 004385.

Isoforms and variants of the IL-6, SELE, FOSB, NRK, NFKB2, FOXP3, ARG1, CEBPDP, TNFα, ADAMTS4, PENK, FOSL1, DUSP1, ACTA1, AGT, ATF3, CDK1, CXCL8, SELP, WAN, TFPI2, NR4A3, genes and gene products can be present in subjects and can be detected, measured, evaluated, and the subjects with such isoforms and variants can be treated by the methods and compositions described herein. Such isoforms and variants can have sequences with between 65-100% sequence identity to a reference sequence, for example with at least at least 65%, at least 70%, at least 80%, at least 90%, at least 95%, at least 96%, at least 97% sequence, at least 98%, at least 99%, or at least 99.5% identity to a sequence described herein or a reference sequence (such as one described in the NCBI or Uniprot databases) over a specified comparison window. Optimal alignment may be ascertained or conducted using the homology alignment algorithm of Needleman and Wunsch, J. Mol. Biol. 48:443-53 (1970).

The present description is further illustrated by the following examples, which should not be construed as limiting in any way.

### Example 1. Materials and Methods

### Methods

### NGS Data Source and Patient Inclusion Criteria

This was an institutional review board (IRB) approved retrospective cohort analysis performed on men with PCa (Protocol Number 190443). Eligible patients in the next generation sequencing (NGS) study had adequate primary tumor tissue sequenced using the CAP/CLIA validated Tempus xT test. Raw RNA-sequencing (RNAseq) data from Tempus was analyzed.

### NGS Gene Expression Analysis and GSEA

Enrichment scores for the tumor samples were calculated using Single-sample Gene Set Enrichment Analysis (ssGSEA) projection (Barbie et al., 2009). Gene expression values were rank-normalized from their absolute expression, and an enrichment score within each tumor sample was calculated by evaluating the differences in the empirical cumulative distribution functions of the genes in the gene set relative to the remaining genes. A positive ssGSEA score indicates a significant overlap of the gene set with groups of genes at the top of the ranked list, while a negative ssGSEA score indicates a significant overlap of the gene set with groups of genes at the bottom of the ranked list. Gene sets from the Molecular Signatures Database (MSigDB) (Liberzon et al., 2011; Liberzon et al., 2015; Subramanian et al., 2005) were used. To quantify the degree of association we used the Information Coefficient (IC) (Kim et al., 2016).

An empirical permutation test was used to compute p-values and assess statistical significance.

### NGS Custom Signature Matrix Generation

Single cell RNA-seq (sc-RNA-seq) data was obtained from Chen et al. (2021), which used 10× genomics sequencing technology to obtain reads from four metastatic and nine localized PCa samples. Using the Seurat single cell analysis package, these samples were arrayed as an expression matrix, normalized, then clustered on 2000 variable marker genes with a resolution setting of 1. Myeloid and T-cell populations were then identified via CD14+ and CD3D/E+(Zheng et al., 2021) expression respectively. These subpopulations were similarly clustered into high resolution subpopulations and identified based on the following marker genes: CD4 Tregs (PMCH, FOXP3) (Zheng et al., 2021; Chen et al., 2018); CD4 Memory Resting (TNFSF14, ATHL1) (Zheng et al., 2021; Chen et al., 2018); Gamma Delta T Cell (DUSP2, CCL4, CD3D) (Zheng et al., 2021; Chen et al., 2018); T Cell Follicular Helper (ICA1, PDCD1) (Zheng et al., 2021; Chen et al., 2018); CD4 Naive (FLT3LG, ANKRD55) (Zheng et al., 2021; Chen et al., 2018); CD4 Memory Activated (IFNG, CCL20) (Zheng et al., 2021; Chen et al., 2018); CD8 T Cell (CD8A, CCL5, CD8B) (Chen et al., 2018); NKT (GZMM, GNLY) (Chen et al., 2018); B Cell Memory (GPR183) (Chen et al., 2018); B Cell Naive (AIM2, GPR183) (Chen et al., 2018); Mast cells (TRIB2, ZNF165, TPSAB1) (Chen et al., 2018); M0 Macrophages (CYP27A1, ACP5) (Chen et al., 2018; Mulder et al., 2021); M1 Macrophages (Mulder et al., 2021; Martinez et al., 2006); M2 Macrophages (TREM2, CD68, HLA-DQA1) (Mulder et al., 2021; Martinez et al., 2006); Monocytes (FCN1, S100A12, FPR1) (Cheng et al., 2021); Dendritic Cells Resting (CD1C, CD1E) (Chen et al., 2018); and Dendritic Cells Activated (CCL22, LAMP3, IDO1) (Chen et al., 2018; Cheng et al., 2021).

A custom signature matrix was generated using these cell type gene expression profiles via the “Create Signature Matrix” function of CIBERSORTx (Steen et al., 2020). This custom signature matrix of pre-metastatic and metastatic prostate immune cell populations was then applied to each of the RNA-seq cohorts to estimate immune cell infiltration via the “Impute Cell Fractions” module from CIBERSORTx.

### NGS Analysis of Immune Cell Infiltration

R statistical software version 4.0.1 was used to measure the correlation between gene expression and immune cell infiltration approximations (R: A language and environment for statistical computing. Version 4.0.1. R Foundation for Statistical computing. R Foundation for Statistical Computing. 2020). R² values and p-values were calculated using the spearman correlation coefficient. Similarly, differences in immune cell infiltration approximations between pre-metastatic and metastatic populations were compared within cohorts and p-values were calculated using the Wilcoxon signed rank test.

### VHA Data Source

Patient information for the clinical cohort was collected from the Veterans Health Administration (VHA) Corporate Data Warehouse (CDW), which contains health records of >9 million veterans from approximately 170 VHA medical centers and 1000 outpatient sites (Affairs USDoV. About VHA. (2021)). This study was reviewed and approved by the VHA San Diego Healthcare System. Waivers of consent and authorization were granted by the Institutional Review Board and the Research and Development Committee of the VHA San Diego Healthcare System (Protocol Number 150169).

### VHA Study Population

Patients in the VHA diagnosed with PCa from 2000 to 2014 were included in this cohort. Follow up ended on Jun. 23, 2017. Patients with unknown initial treatment or clinical staging were excluded from the cohort.

### VHA Measures

Information on age at diagnosis, race, employment status, Gleason scores, T stage, and metastatic stage was collected from the Veterans Affairs Informatics and Computing Infrastructure (VINCI) CDW Oncology Registry. Pre diagnostic prostate specific antigen (PSA) levels were collected from the VINCI Prostate Cancer Data Core to use as baseline nearest PSA level before diagnosis.

Outcomes of interest included associations of pre diagnostic treatment with any TNFα antagonist use (adalimumab, certolizumab, erelzi, golimumab, etanercept) with PCa characteristics at diagnosis (Gleason score, clinical stage, and PSA), and long-term development of metastatic disease. Diagnosis of metastatic disease was obtained through the Prostate Cancer VINCI Data Core (Alba et al., 2021), which uses an internally developed Natural Language Processing (NLP) tool to identify cases of metastases in PCa patients. Time to event endpoints were calculated from the date of diagnosis to the event of interest or censored at the date of last follow up. Patients who died without experiencing an event were censored at the time of death in Cox proportional hazards models and counted as a competing event in cumulative incidence functions.

### VHA Statistical Analysis

Univariable cumulative incidences of development of metastases were measured. Logistic regression models were used to measure associations between sociodemographic characteristics at time of diagnosis and TNFα antagonist use. Age at diagnosis, African American ethnicity, and employment status at diagnosis were additional covariates for models with these outcomes: presenting with Gleason 8 or higher disease, presenting with T stage (3 or 4 vs. 1 or 2), presenting with a PSA>20 ng/mL, and presenting with metastatic disease at diagnosis. Cox proportional hazards models controlling for the same variables were used to measure associations between pre diagnostic TNFα antagonist use and development of metastases.

### Results

### PCa Localized Versus Metastatic RNA-Seg Analysis

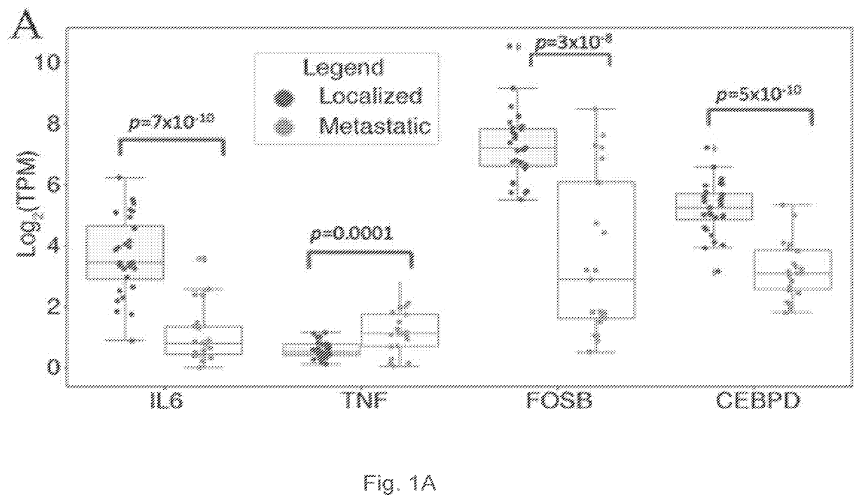

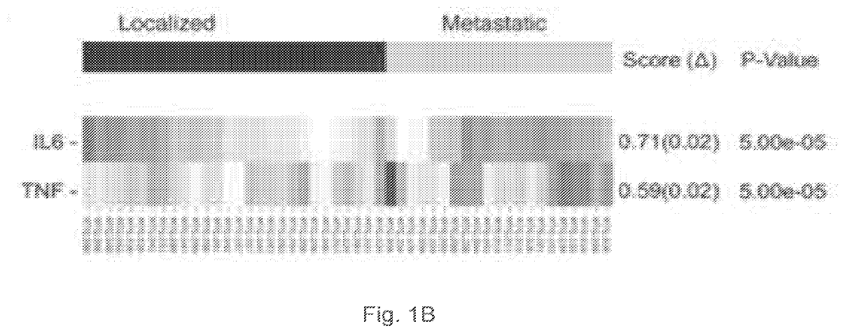

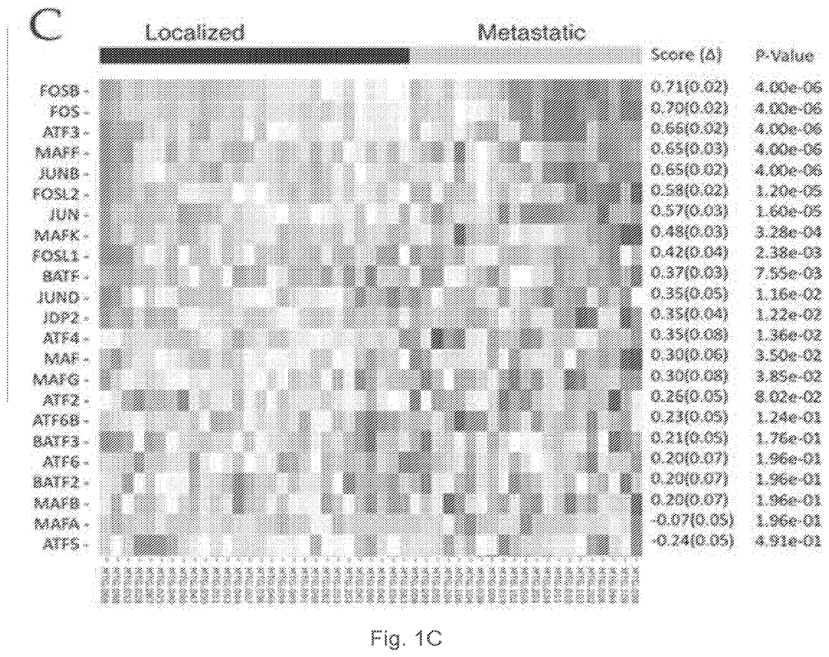

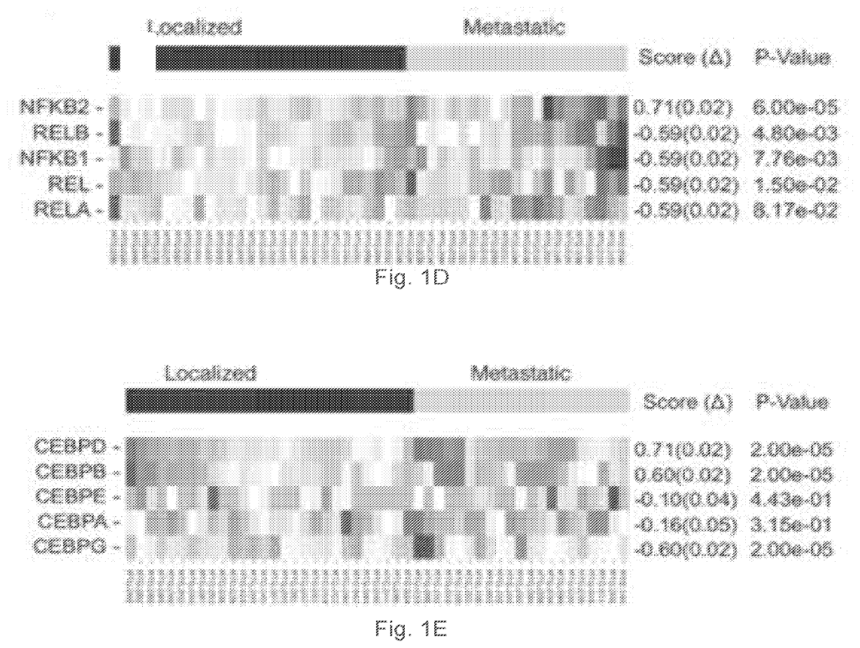

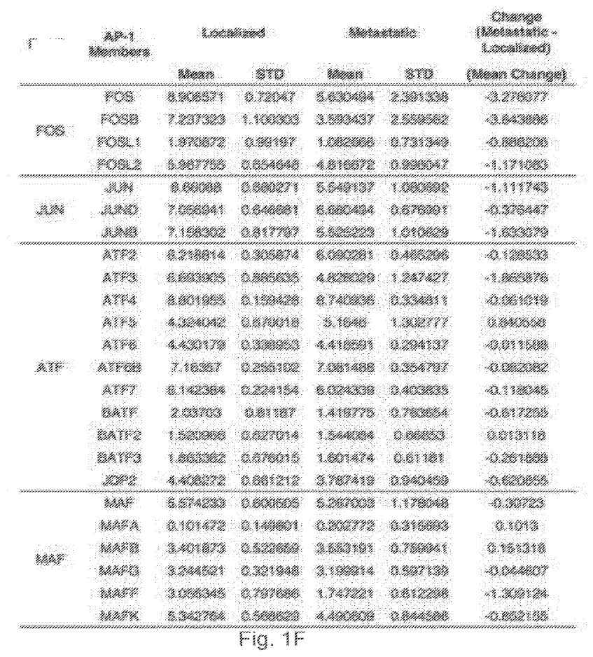

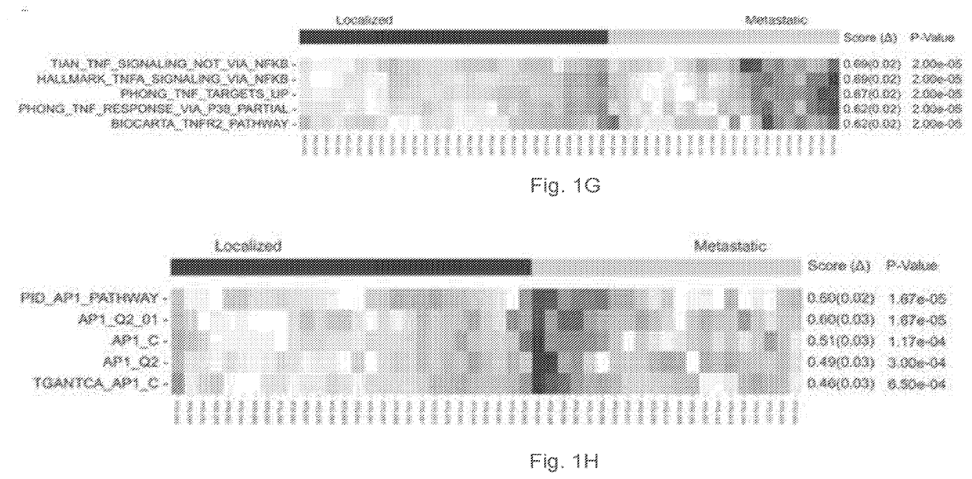

Somatic tumor RNA-seq data for expression levels of TNFα and IL-6 in localized versus metastatic disease (FIGS. 1A and 1B) was obtained. Elevated TNFα expression levels were observed in metastatic disease (localized mean=0.0557 vs. metastatic mean=1.244 Log₂TPM, p=0.0001, FIG. 1A). IL-6 levels, in contrast, revealed a significant expression reduction in metastatic compared to localized disease (mean=3.652 vs. mean=1.101 log₂TPM, p=7×10⁻¹⁰, FIG. 1A). This result is demonstrated in a TNFα and IL-6 expression heatmap, as lower relative expression of IL-6 correlates with higher relative expression of TNFα, and vice versa, regardless of the inherent cohort heterogeneity (FIG. 1B).

The upstream transcriptional factors (TFs) associated with TNFα and IL-6 regulation such as the Activator Protein 1 (AP-1) family of TF's, NFkB and CEBP were examined to decipher molecular mechanisms that may drive the differential expression of these cytokines in localized versus metastatic disease (FIGS. 1C-1F). It was found that AP-1 TF FOSB had a particularly significant association with IL-6 expression (FIGS. 1A, 1F). Other AP-1 TF's such as FOS, ATF3, JUNB and CEBP binding protein CEBPD are also correlated with IL-6 expression but to a lesser extent than FOSB (FIGS. 1A, 1C, 1F). The structure of AP-1 is that of a homo- or heterodimer composed of proteins representing FOS, JUN and ATF sub families (Chinenov & Kerppola, 2001; Shaulian & Karin, 2001). The dimerization specificity among various AP-1 TFs defines downstream target functional outcomes by binding to gene promoters that include IL-6, TNFα, SELE and many other genes (Vartanian et al., 2011; Harwood et al., 2000; Kim et al., 2007). Thus, the decrease of FOSB in metastatic disease may cause divergence between TNFα and IL-6 PCa expression levels by changing the available ratios of the corresponding translated proteins for dimerization.

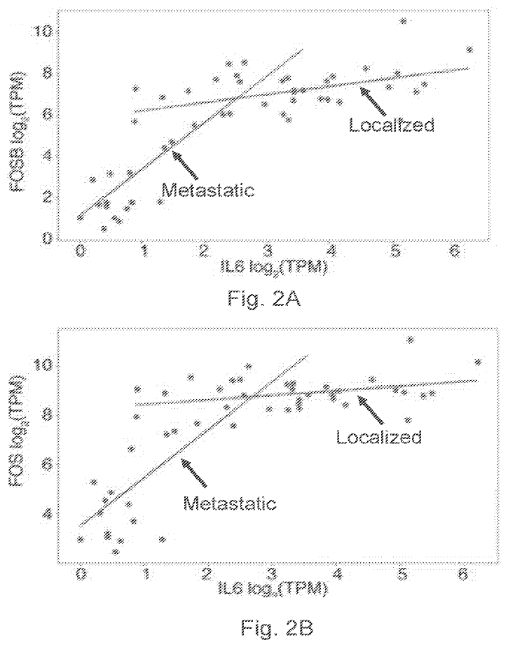

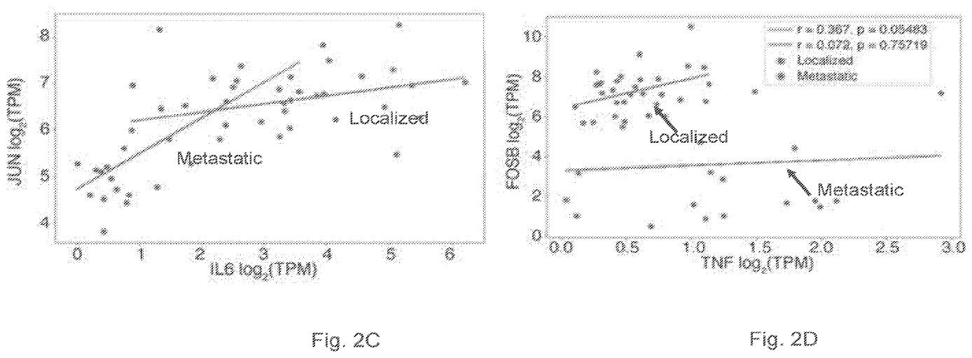

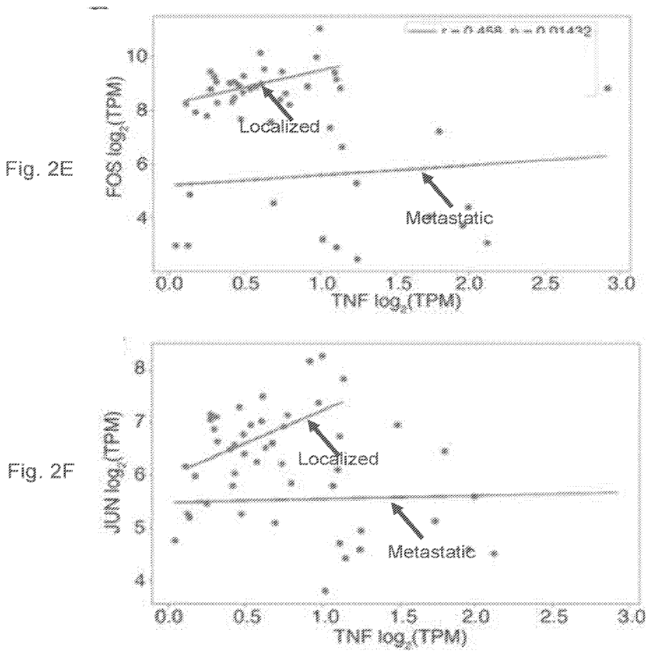

To further explore the association between TNFα, IL-6 and AP-1 FOSB, FOS and JUN standard correlation analysis was performed and a significant linear correlation between IL-6 and FOSB (r=0.79, FIG. 2A), IL-6 and FOS (r=0.73, FIG. 2B) was observed as well as IL-6 and JUN (r=0.64, FIG. 2C) in the metastatic state. In localized disease FOSB displayed a modest linear correlation with IL-6 (r=0.451, p=0.016. FIG. 2A). However, TNFα did not show significant association with FOSB, FOS or JUN in the metastatic setting (FIGS. 2D-2F). A weaker linear correlation was observed for all three AP-1 TFs with TNFα in localized disease (FIGS. 2D-2F). It was also observed that TNFα and IL-6 are neither correlated significantly with each other in localized nor in metastatic groups (FIG. 2G).

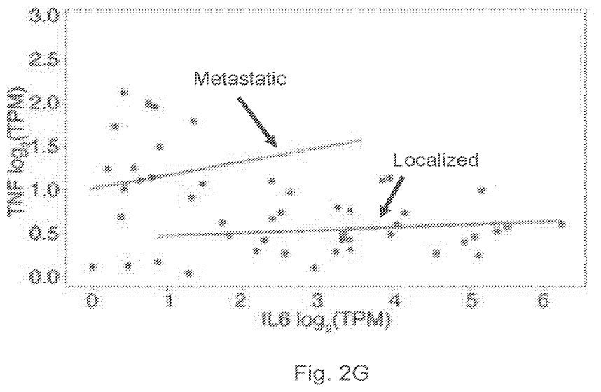

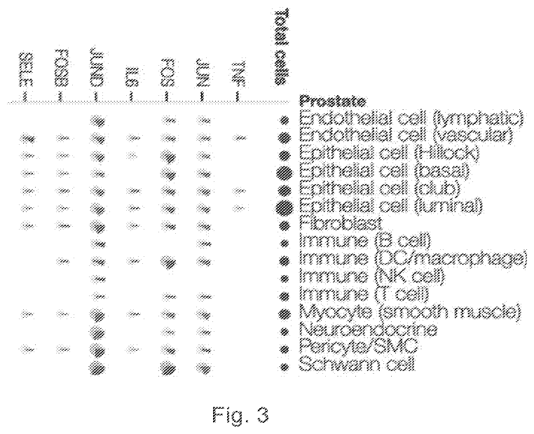

To better assess the significance of TNFα and IL-6 differential expression in PCa progression, their expression was analyzed in normal prostate tissue. The publicly available prostate Genotype-Tissue Expression (GTEx) sc-RNA-seq data was accessed for TNFα, IL-6 and AP1 expression (FIG. 3) (Consortium G, (2013). GTEx TNFα expression is limited to luminal, club epithelial and vascular endothelial cells but not detectable in immune cells (FIG. 3). However, IL-6 as well as FOSB were expressed in a wider variety of cells including immune cells (FIG. 3).

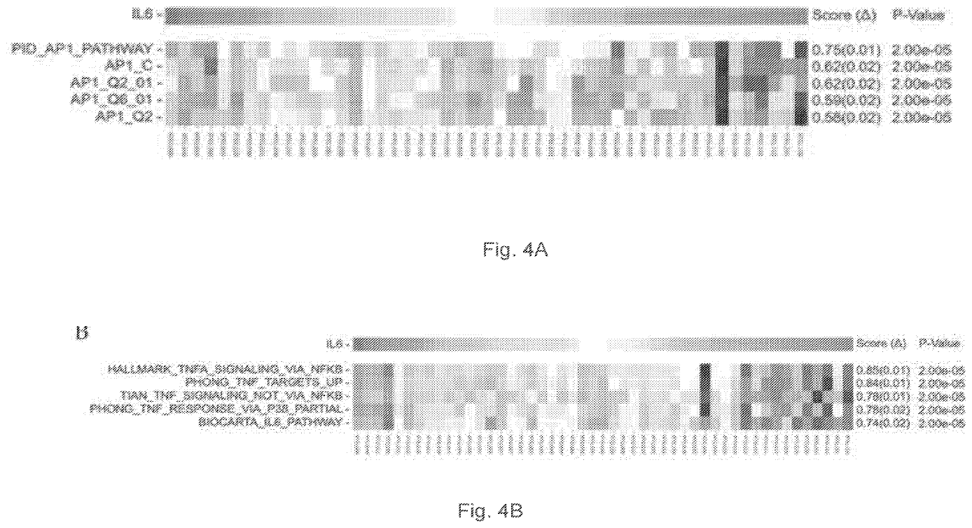

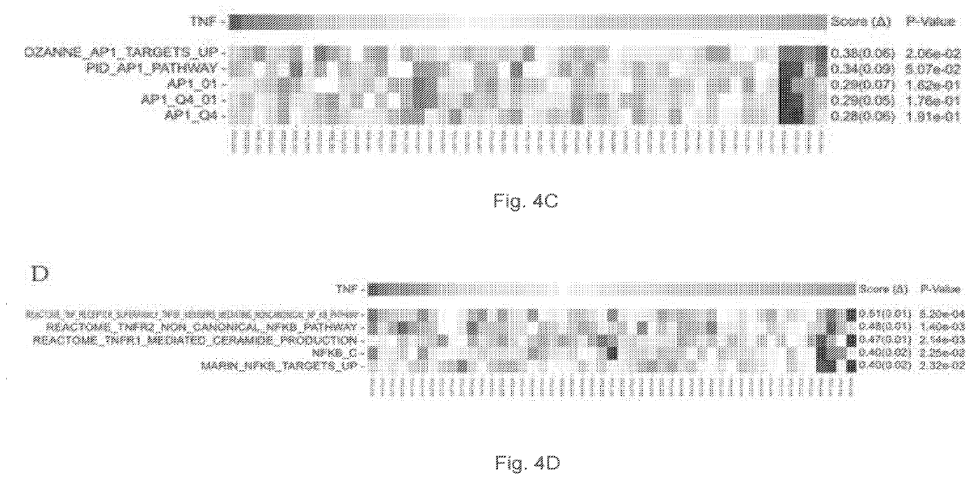

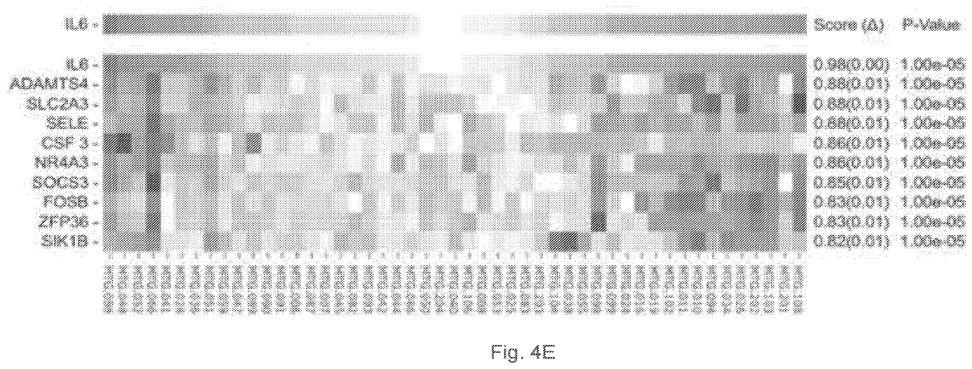

It was established that TNFα and IL-6 expression in PCa appears to diverge from that of normal prostate tissue, therefore the regulatory signaling pathways implicated in their function was explored. To that end ssGSEA analysis (Barbie et al., 2009) was performed, where the patient mRNA profiles were projected onto the space of MSigDB (Liberzon et al., 2011; Liberzon et al., 2015) gene sets and the ssGSEA profiles matched against mRNA expression profiles of TNFα and IL-6 using as measure of association the IC (see “Methods”). When matching gene set profiles against IL-6 (mRNA), among the top scoring gene sets were those representing AP-1 and NFKB signaling pathways implicated in TNFα regulation. The top scoring gene sets include a gene set representing the AP-1 transcription factor network (PID_AP1_PATHWAY, FIG. 4A) and the MSigDB hallmark that represents genes regulated by NF-kB in response to TNFα (HALLMARK_TNFA_SIGNALING_VIA_NFKB, FIG. 4B). However, the top scoring gene sets for TNFα (mRNA) produced a gene set representing cancer motility and invasion genes up-regulated by the AP-1 transcription factor (Ozanne_API_TARGETS_UP, FIG. 4C), and one representing TNF receptor superfamily (TNFSF) members mediating the non-canonical NF-kB pathway (REACTOME_TNF_RECEPTOR_SUPERFAMILY_TNFSF_MEMBERS_MEDIATI NG_NONCANONICAL_NF_KB_PATHWAY, FIG. 4D). As expected, the top scoring ssGSEA results for IL-6 expression are also aligned with localized disease (FIGS. 1G, 1H). They differed for the NFKB pathway where ssGSEA found association with PCa disease progression (TIAN_TNF_SIGNALING_NOT_VIA_NFKB, FIG. 1G).

### E-Selectin is Downregulated in PCa Metastatic Disease

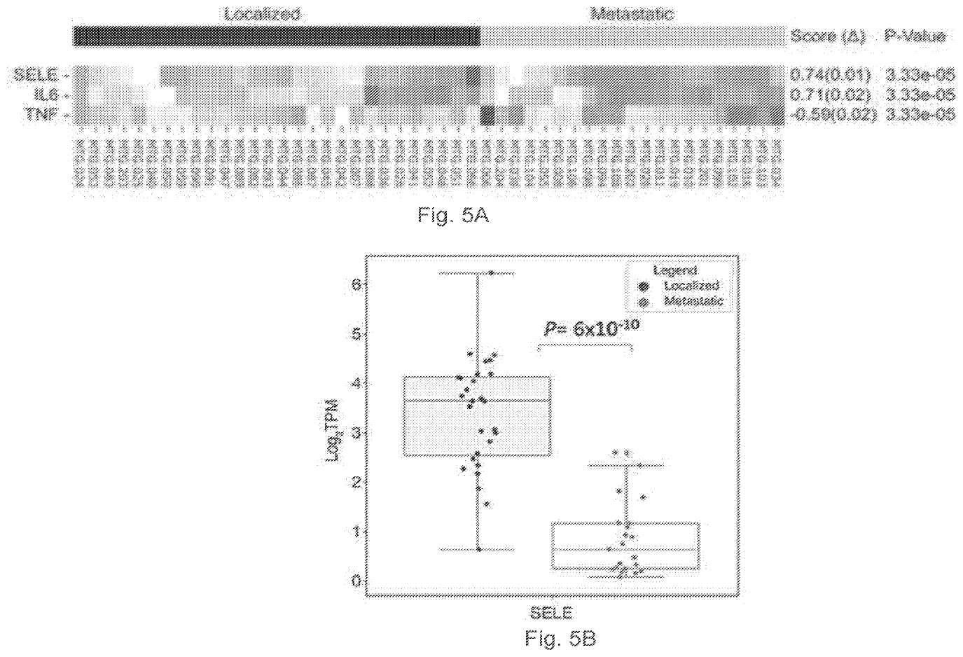

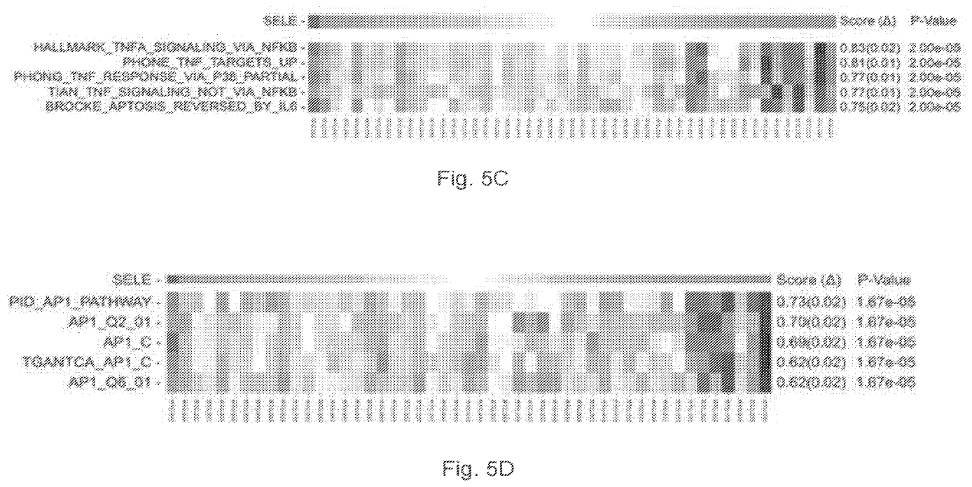

To identify other factors possibly associated with IL-6 mediated effects on the tumor immune microenvironment (TME), the top differentially expressed genes between localized vs. metastatic disease were analyzed and their correlation with IL-6 evaluated. 9 genes were found that were differentially expressed and correlated with IL-6 levels across samples (FIG. 4E). Among these, 2 had previously been implicated in modulation of immune activities: e-Selectin (SELE) and ADAMTS4. SELE is implicated in recruitment of leukocytes and is associated with inflammation (Robbins et al., 1999). It can mediate adhesion of tumor cells to endothelial cells to promote cancer metastasis (Dimitroff et al., 2005). SELE expression was analyzed in the patient cohort (FIG. 5A) and observed significant downregulation of this gene in metastatic disease (localized mean=3.381, metastatic mean=0.881, p=6×10⁻¹⁰, FIG. 5B).

ssGSEA analysis was conducted for SELE, and similar top scoring gene sets as in the results of IL-6, including the AP-1 favored PID_AP1_PATHWAY and NFKB was most associated with HINATA_NFKB_TARGETS_FIBROBLAST_UP (FIGS. 5C, 5D), were found.

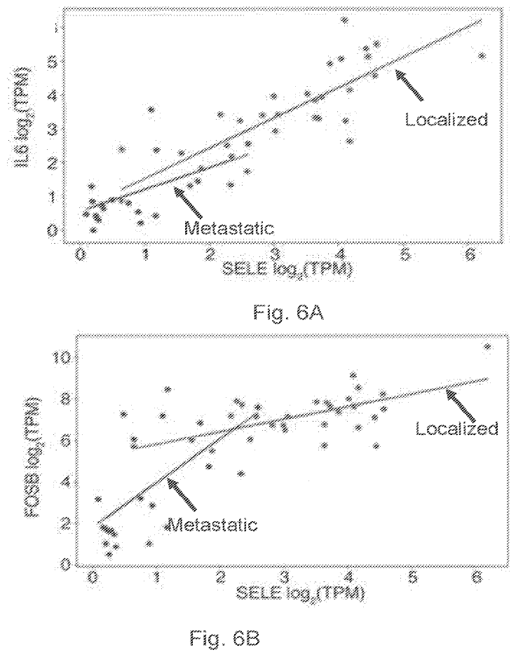

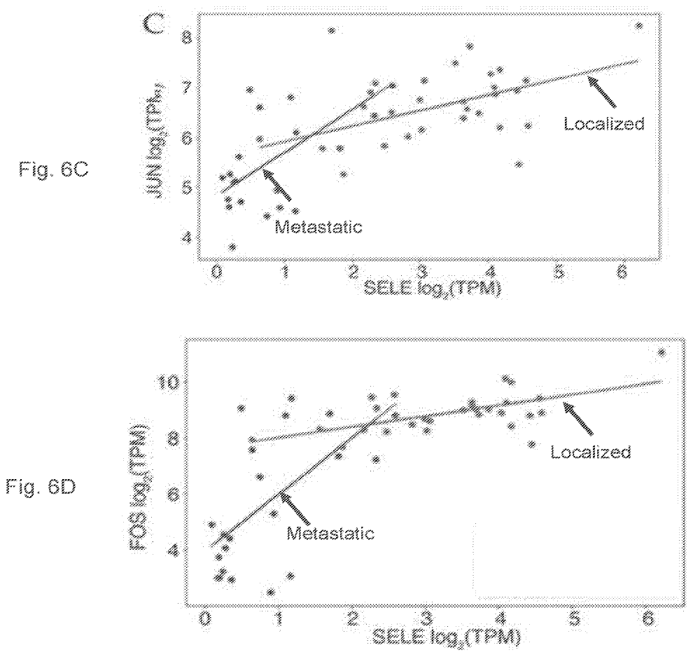

A Pearson correlation analysis was performed to test the association of SELE expression with AP-1, TNFα and IL-6 (FIG. 6). A strong linear correlation was observed between SELE with IL-6 in both localized and metastatic states, however the correlation is stronger in the localized state (FIG. 6A). FOSB, JUN, and FOS also show a significant correlation with SELE expression (FIGS. 6B-6D) which is not surprising since AP-1 constitutes part of the SELE promoter.

### PCa Immune Microenvironment

The PCa immune microenvironment while considered “cold” due to its limited response to immunotherapy has not been fully elucidated. The pro-inflammatory cytokines TNFα and IL-6 as well as SELE are central to immune response regulation and have been implicated as potential targets for PCa therapy. IL-6 antagonists were tested in a clinical setting but failed due to lack of efficacy (Fizazi et al., 2012). There is an ongoing drug development effort in PCa using uproleselan (GMI-1271), a SELE antagonist (Muz et al., 2021). By analyzing patient bulk RNA-seq data it was observed that IL-6 and SELE expression are downregulated in metastatic disease, thus possibly contributing to an immunosuppressive effect (FIGS. 4E, 5A). However, TNFα expression increases with disease progression suggesting that regulatory T cells are not engaged in maintaining immune homeostasis to suppress excessive immune responses. Interestingly, whereas enhanced TNFα expression was observed in the metastatic setting, this was associated with downregulation of classical TNFα signaling by ssGSEA analysis (FIG. 1G). This result indicates that TNFα may act in the metastatic setting through noncanonical pathways or on a wider cadre of target cells than previously recognized.

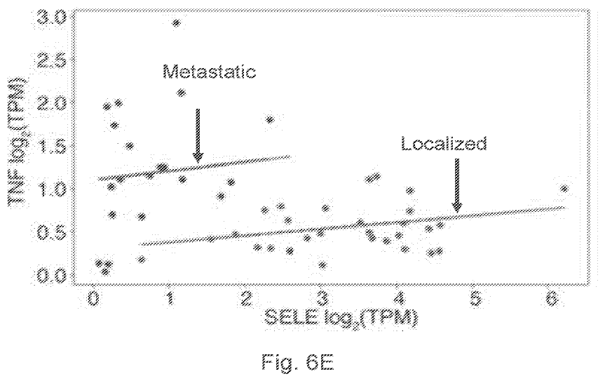

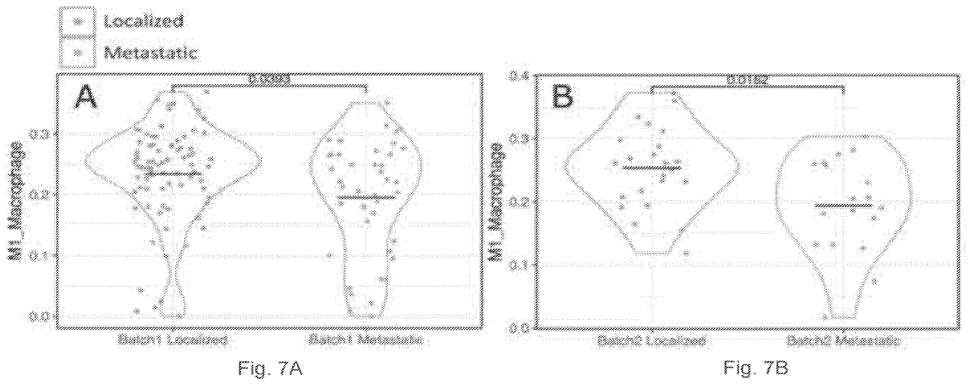

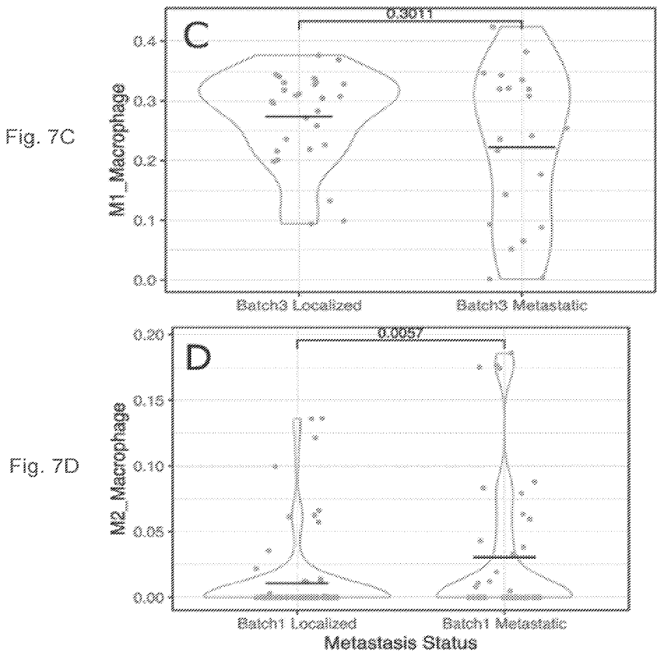

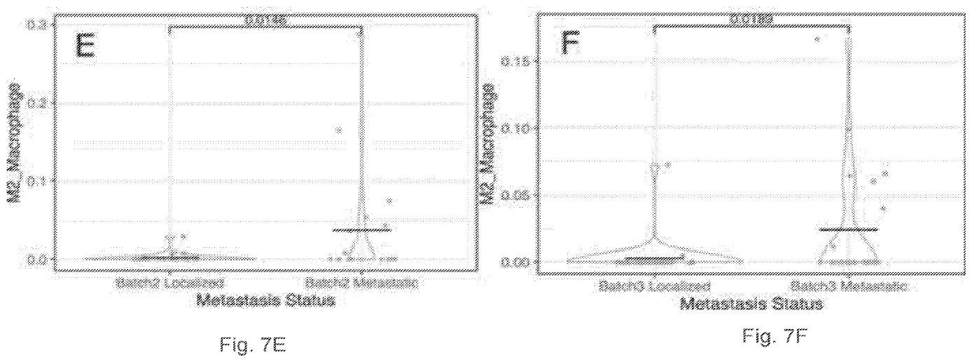

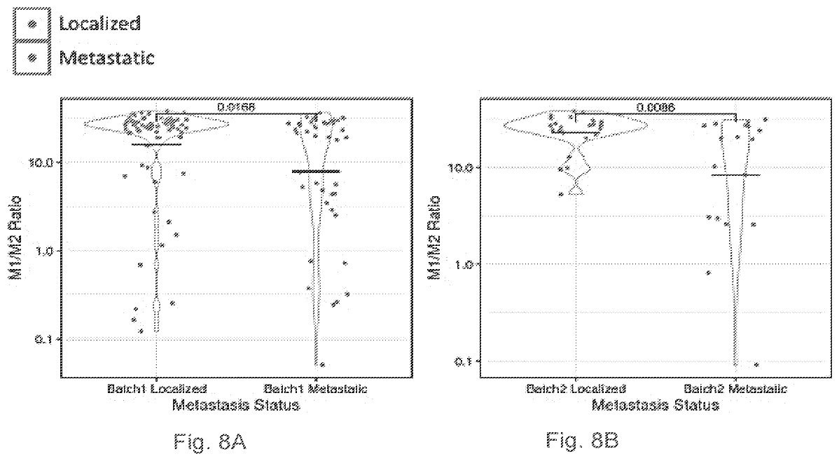

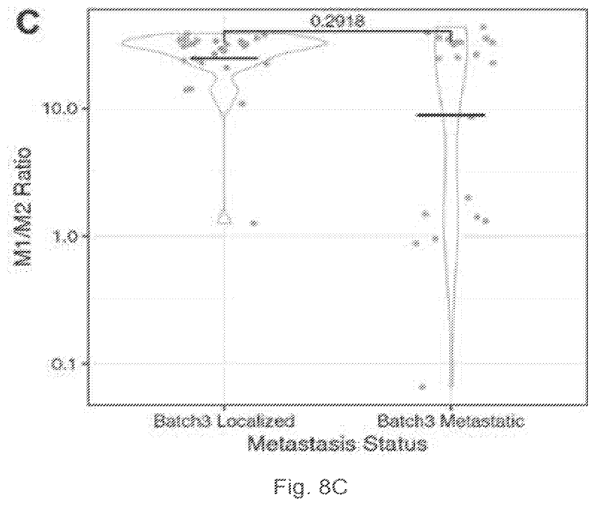

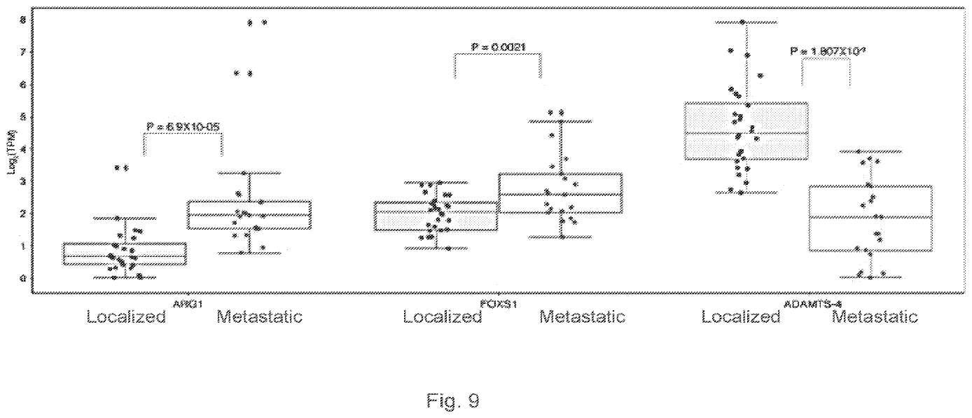

There is experimental and clinical evidence demonstrating that the pro-inflammatory effect of TNFα can switch to an immunosuppressive function after prolonged exposure (Clark et al., 2005; Kollias et al., 2002; Ye et al., 2020). To assess the composition of the infiltrating immune cells types, immune cell infiltration estimates were generated using a custom gene signature matrix derived from an annotated sc-RNA-seq dataset of localized and metastatic PCa samples (Table 1). While a wide range of infiltrating immune cells was found, the most significant differences were found for M1 and M2 macrophages (Table 1). Localized disease generally had more M1 macrophages and fewer M2 macrophages than metastatic disease (Table 1, FIG. 7A). To validate this result, additional RNA-seq patient datasets were accessed and it was found that M2 macrophage enrichment in metastatic disease was reproducible in all study cohorts (FIGS. 7D-7F) while the M1 result reproduced in one additional cohort and trended in the other (FIGS. 7A-7C). Metastatic tumors more often showed reduced M1/M2 macrophage ratios than localized tumors (FIG. 8) and expressed higher levels of ARG1 and FOXS1 which are both associated with M2 macrophage polarization (FIG. 9) (Colegio et al., 2014; Arlauckas et al., 2018; Vadevoo et al., 2021; Liu et al., 2022).

### Immune Sequela of Extracellular Matrix (ECM) Remodeling

Among the genes highly correlated with IL-6 expression, ADAMTS-4 showed the highest association (FIG. 4E). ADAMTS-4, a member of the ADAMTS family of metalloproteinases, was highly expressed in localized samples compared to metastatic disease (localized mean=4.83, metastatic mean=2.31, p=1.8×10⁻⁹, FIG. 9). A major substrate of ADAMTS-4 is the large aggregating extracellular matrix (ECM) proteoglycan versican (VCAN) (Papadas & Asimakopoulos, 2020).

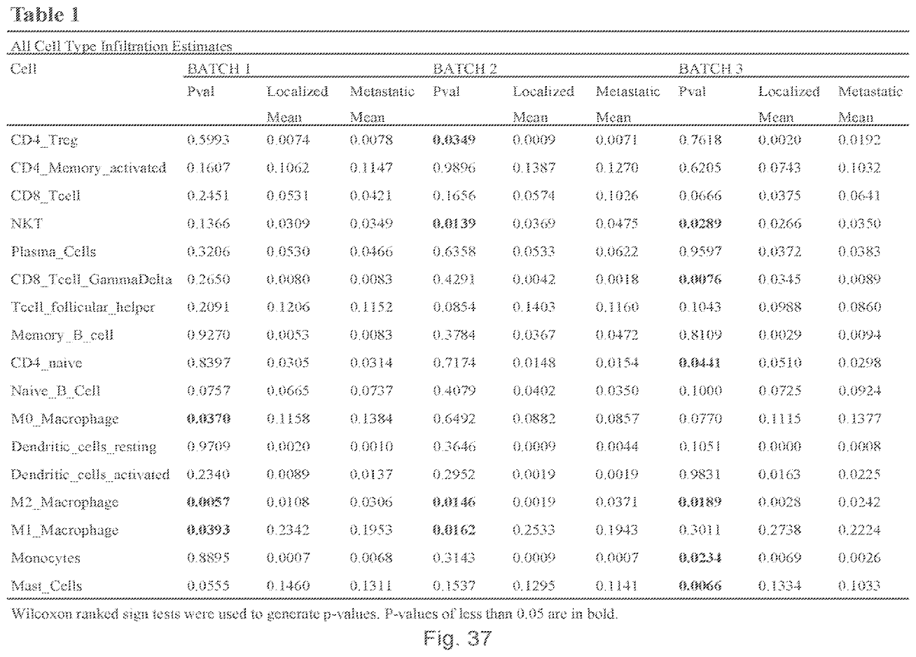

VCAN has major functions in tumor cell growth and metastasis, and in our analysis it is expressed robustly both in localized disease and in metastatic samples, underscoring its likely importance along the entire tumor natural history. From the immune perspective, VCAN has been credited with immunoregulatory functions: it acts through Toll-like receptor 2 (TLR2) to dampen antigen presentation by tumor-infiltrating dendritic cells (Tang et al., 2015). However, an N-terminal proteolytic fragment of VCAN, versikine, arising through the actions of ADAMTS-4 and other versicanases, conversely regulates cross-presenting type 1 conventional dendritic cell (cDC1) abundance and activation in the TME, to promote immune cell trafficking to the tumor and effector priming (Papadas et al., 2022). The data suggest that ADAMTS-4 may contribute to the immune activity in localized tumors through VCAN proteolysis and cDC1 regulation. The increased expression of the master lineage regulator of cDC1, Batf3, as well markers of immune infiltration such as CXCR3 in localized disease, are in agreement with this hypothesis (FIG. 1C). Table 1 (FIG. 37) shows infiltration estimates in metastatic vs. pre-metastatic samples across all cohorts.

During metastatic progression, attenuated expression of ADAMTS-4, e.g., through TGFb (Cross et al., 2005), dampens the moderating effects of versikine on the immunoregulatory activities of non-proteolyzed parental VCAN.

### Anti-TNF Effect in PCa Patients

TNFα is expressed in PCa throughout disease progression and this pattern could be associated with immune remodeling. TNFα chronic expression is a hallmark of autoimmune disease (AI) widely treated with TNFα antagonists. Studies have shown men with AI have a higher risk of all urologic cancers, including bladder, prostate, and kidney cancers (Liu et al., 2013), and higher incidence of PCa than those without AI diseases. Thus it was conjectured that administration of a TNFα antagonist may confer therapeutic benefit in PCa. A VHA patient registry study was performed to determine the clinical characteristics associated with anti-TNFα therapeutic outcomes. Specifically, the associations of TNFα antagonist administration prior to PCa diagnosis was investigated. The study cohort included 120,204 PCa patients from VHA CDW, among them 390 had TNFα antagonist therapy prior to PCa diagnosis. The cohort was binarized into TNFα naïve patients (group 1, n=119,814) and those who received TNFα antagonist therapy prior to being diagnosed with PCa (group 2, n=390, Table 2). The mean age at diagnosis was higher in group 1 (65.76 years vs. 64.96, p=0.052, Table 2). Patients in group 2 were significantly more likely to be diagnosed with T1 disease than those in group 1 (73.3% vs. 65.9%, p=0.002, Table 2). Patients in group 2 were less likely to be African American (White: 82.8% vs 69.2%, African American 14.4% vs 27.3%, p<0.001, Table 2).

TABLE 2
Demographics and baseline disease characteristics of study population

| | No TNFα use | | TNFα use pre PCa | | p |
|---|---|---|---|---|---|
| | (Group 1) | | diagnosis (Group 2) | | Value |
| Number | 119814 | | 390 | | |
| Age at Dx [mean(SD)] | 65.76 | (8.14) | 64.96 | (6.85) | 0.052 |
| Less than 55 | 8379 | (7.0%) | 20 | (5.1%) | 0.179 |
| 55-64 | 49546 | (41.4%) | 174 | (44.6%) | 0.21 |
| 65-74 | 43316 | (36.2%) | 159 | (40.8%) | 0.066 |
| 75 and Up | 18573 | (15.5%) | 37 | (9.5%) | 0.001 |
| Race | | | | | <0.001 |
| African American | 32655 | (27.3%) | 56 | (14.4%) | |
| White | 82894 | (69.2%) | 323 | (82.8%) | |
| Other | 1318 | (1.1%) | 6 | (1.5%) | |
| Unknown | 2947 | (2.5%) | 5 | (1.3%) | |
| T Stage 1 | 78899 | (65.9%) | 286 | (73.3%) | 0.002 |
| T Stage 2 | 36839 | (30.7%) | 98 | (25.1%) | 0.019 |
| T Stage 3 or 4 | 4075 | (3.4%) | 6 | (1.5%) | 0.059 |
| Gleason 6 | 48261 | (40.3%) | 172 | (44.1%) | 0.138 |
| Gleason 7 | 47878 | (40.0%) | 162 | (41.5%) | 0.56 |
| Gleason 8 or Higher | 23690 | (19.8%) | 56 | (14.4%) | 0.009 |
| Mean pre diagnostic | 18.15 | (71.59) | 8.60 | (11.99) | 0.011 |
| PSA (SD) | | | | | |
| Median pre diagnostic | 6.55 | | 5.5 | | |
| PSA | | | | | |
| PSA over 20 at Dx | 12253 | (11.4%) | 23 | (6.3%) | 0.003 |
| N Stage 1 | 1965 | (1.6%) | 6 | (1.5%) | 1 |
| M Stage 1 | 4840 | (4.0%) | 9 | (2.3%) | 0.108 |

When performing logistic regression, prior TNFα antagonist use (group 2) was associated with reduced odds of presenting with Gleason 8 or higher scores [Odds Ratio (OR): 0.690, p=0.011, Table 3] and reduced odds of presenting with T stage 3 or 4 disease (OR: 0.447, p=0.056). Additionally, group 2 showed reduced odds of having PSA over 20 ng/mL at diagnosis (OR 0.572, p=0.010, Table 3). When measuring associations with metastatic disease at presentation, TNFα antagonist use trended towards an association with reduced metastases at diagnosis, but this was not statistically significant (OR: 0.581, p=0.108, Table 3).

TABLE 3
Associations between group 2 and disease characteristics at PCa diagnosis
from multivariable logistic regression models

| | Gleason 8 or | | T Stage 3 or 4 at | | PSA Over 20 at | | Metastases at | |
|---|---|---|---|---|---|---|---|---|
| | higher at diagnosis | | diagnosis | | diagnosis | | diagnosis | |
| | Odds | | Odds | | Odds | p | Odds | p |
| Outcome | ratio | p Value | ratio | p Value | ratio | Value | ratio | Value |
| Group 2 | 0.690 | 0.011 | 0.447 | 0.056 | 0.572 | 0.010 | 0.581 | 0.108 |
| AA Race | 1.03 | 0.044 | 0.997 | 0.95 | 1.75 | <0.001 | 1.16 | <0.001 |
| Age >65 years | 1.67 | <0.001 | 1.42 | <0.001 | 1.92 | <0.001 | 1.92 | <0.001 |
| Employed at | 0.821 | <0.001 | 0.806 | <0.001 | 0.677 | <0.001 | 0.685 | <0.001 |
| diagnosis | | | | | | | | |

PSA prostate specific antigen, AA African American

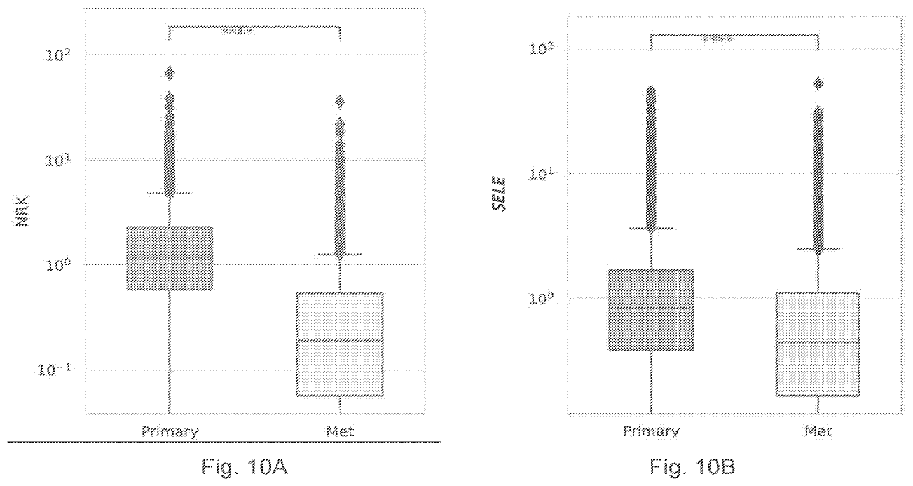

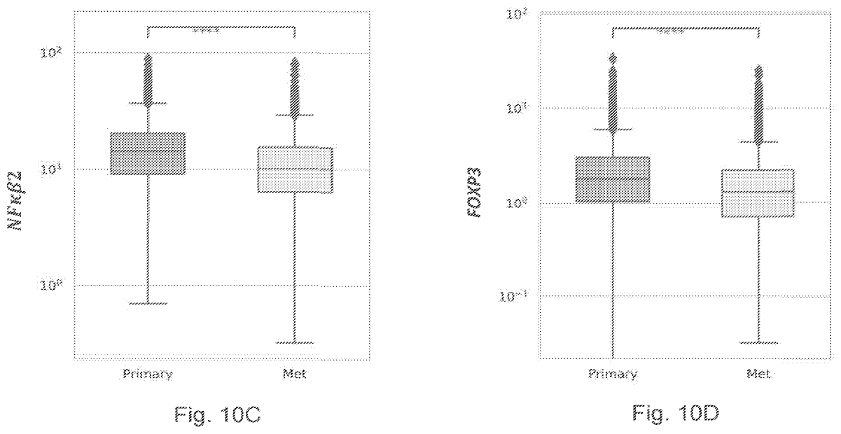

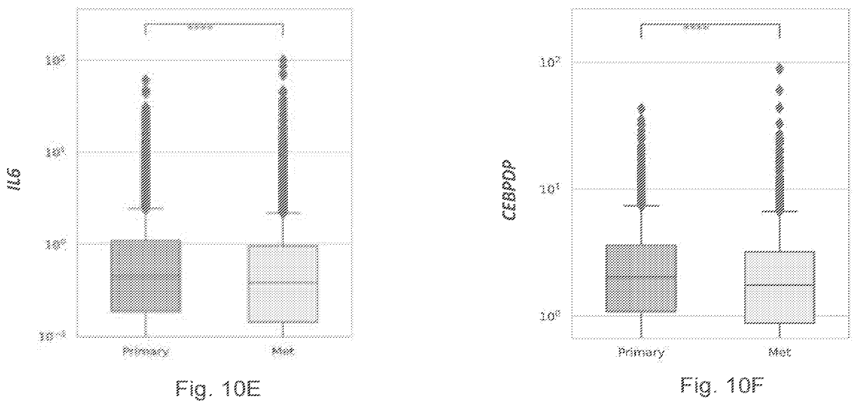

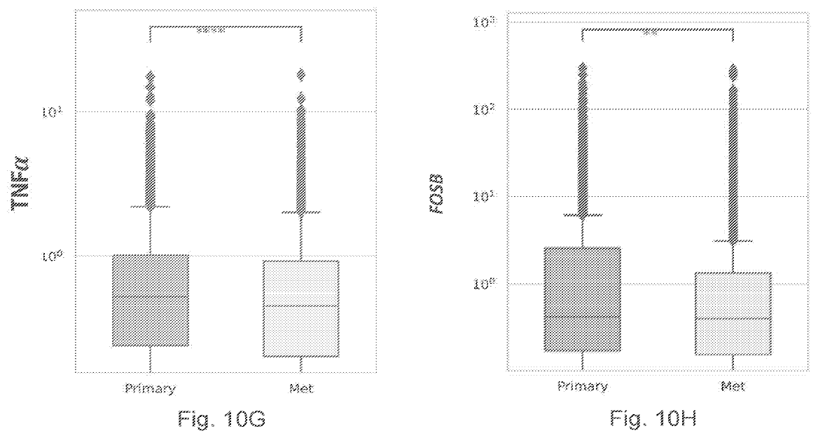

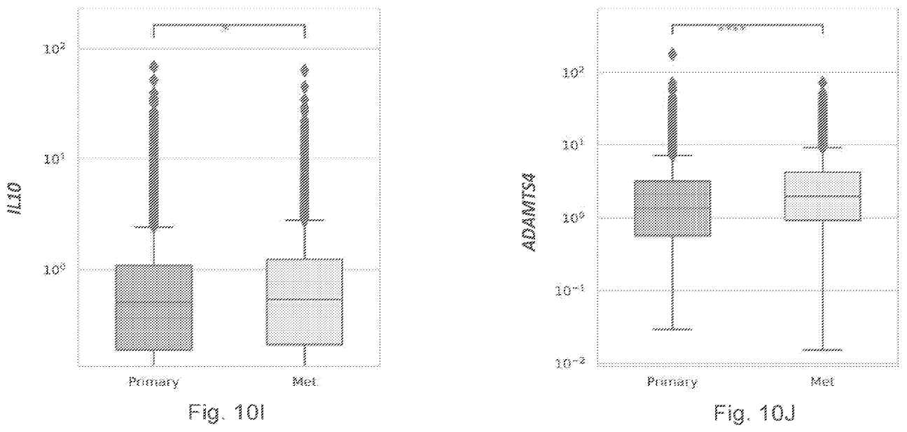

Cumulative incidences of metastases at ten years between the two groups were 13.4% for those in group 1 and 8.9% for those in group 2 (p=0.135, FIG. 10). Cox proportional hazards models were applied and it was found that group 2 was not associated with long term development of metastases [Hazard Ratio (HR) 0.79, p=0.19, FIG. 10].

### Discussion

A major obstacle for conducting clinically relevant PCa research has been the lack of cell lines and in vivo experimental models that closely represent human disease progression. To overcome this hurdle, a discovery platform enabling investigation of differential gene expression associated with disease progression was developed.

The data herein has implications for understanding the immune and stromal context of PCa progression and metastasis. Whereas both IL-6 and TNFα have pleotropic and stage-specific functions, both cytokines have been implicated in the orchestration of the pre-metastatic niche (Kim et al., 2009). The data highlights an unexpected discrepancy between increased TNFα expression in metastatic samples and reduced enrichment (ssGSEA) of TNFα canonical signatures. This discrepancy may suggest that TNFα promotes metastatic disease through signaling that is distinct from the classical pro-inflammatory pathways triggered by this cytokine.

TFs of AP-1, regulating TNFα, IL-6 and SELE are implicated as oncogenes or tumor suppressors in many cancers (Eferl & Wagner, 2003; Ozanne et al., 2007; Jochum et al., 2001) with drug development programs targeting cJun, JunB, JunD, cFos, FosB, Fra1 and Fra2 (Brennan et al., 2020). The majority of these studies reported upregulations of AP-1 family members. However, in line with finding, downregulation of AP-1 TFs has been reported in prostate, gastric, ovarian, colon, cervical and other cancers (Brennan et al., 2020). Furthermore, downregulation of JUNB/AP-1 in PCa progression was reported by MK Thomsen et al. (2015). By focusing on TNFα, IL-6 and SELE function in inflammation we found evidence linking FOSB to PCa disease progression and identified FOSB/AP-1 as a gate keeper. In designing therapeutic intervention targeting FOSB it will be important to address that FOSB function is stage and context specific.

At the level of immune involvement, comparison of the immune cell repertoire between localized and metastatic disease reveals a preponderance of M1 (inflammatory macrophages) in the former transitioning into alternatively activated (M2) macrophages in the latter. This observation suggests that in the primary setting, cancers arising within the physiological structure of the prostate gland are characterized by adaptive immunity that likely favors local growth and propagation of the cancer. Local IL-6-driven inflammation may have direct growth effects on the cancer cells and indirect tumor-promoting effects on the bone marrow microenvironment (e.g., through tolerogenic polarization of antigen-presenting cells). Indeed, earlier work has shown IL-6 to directly promote the growth of prostate carcinoma cells (Nguyen et al., 2014). It is therefore likely that localized cancer arising within the native prostate tissue benefits from sustained local inflammatory networks driven by IL-6, TNFα and SELE. The data suggest that the inflammatory context radically changes in the setting of non-native metastatic tissue where alternatively activated macrophages pre-dominate. In the metastatic setting, the emphasis shifts from growth promotion (since relatively growth-independent variants have escaped selection and metastasized) to immune evasion, tissue remodeling and angiogenic support. All the latter attributes have been associated in earlier studies with M2 macrophages (Allavena et al., 2008). Indeed, more recent studies support this hypothesis and show that tumor-associated M2 macrophages, as well as markers of angiogenesis and lymph angiogenesis, predict the prognosis of patients with non-small cell lung cancer (Hwang et al., 2020).

These transitions in immune repertoire between primary and metastatic disease reflect changes in stromal remodeling and its cross talk with anti-cancer immunity. ADAMTS-4, a known target of TGFB immunosuppressive signaling, is downregulated in metastasis. ADAMTS-4 cleaves the immunomodulatory matrix proteoglycan VCAN. In its intact form, VCAN acts on antigen-presenting cells, dendritic cells and macrophages, to dampen tumor antigen presentation and immune responses. However, a bioactive N-terminal fragment, versikine, arising through the activities of ADAMTS-4 and other versicanases, promotes the abundance and activity of tumor antigen cross-presenting cDC1 subset. Indeed, the relative overexpression of the cDC1 master regulator, Batf3, seems to corroborate this hypothesis. cDC1 are key orchestrators of a “hot” immune microenvironment through chemokine networks that drive T-cell infiltration into the tumor (Spranger et al., 2017).

The increased expression of CXCR3, the receptor for T-cell chemoattractant chemokines CXCL9 and CXCL10 in localized disease appears consistent with enhanced immune milieu of localized specimens. M1 macrophages and immunogenic DC subsets are essential for sufficient local production of CXCL9/10 that drive T-cell-mediated inflammation (Reschke & Gajewski, 2022).

In a recent study it was shown that patients with benign prostatic hyperplasia (BPH) and AI who received TNFα antagonists prior to BPH diagnosis were less likely to develop BPH (Vickman et al., 2022). Importantly, methotrexate did not have this effect, further implicating TNFα as a viable target in BPH (Vickman et al., 2022). The RNA-seq patient data analysis implicates TNFα as a potential target in PCa. To evaluate the clinical implications of this finding the data of 120,204 PCa patients from VHA CDW was analyzed for outcomes associated with anti-TNFα treatment prior to PCa diagnosis. A significant association with earlier grade and stage disease at diagnosis and a trend for improved metastatic propensity was observed.

Taken together, the data demonstrates clear differences in immune contexture between localized and metastatic disease in PCa. Primary localized disease demonstrates features of local inflammation and adaptive immunity, likely counterbalanced by immune checkpoint-driven T cell exhaustion and/or defects in antigen presentation. By contrast, metastases demonstrate immune cold microenvironments and a shift towards resolution of inflammation and tissue repair. The data provide novel insights into the potential mechanisms accounting for the modest efficacy of immune checkpoint inhibitors in advanced PCa and suggest that combinations of immunotherapy with anti-angiogenic or stroma-modifying therapy may improve patient outcomes. In this context, clinical trials with antiangiogenic agents such as bevacizumab yielded conflicting results in the treatment of PCa (Ferrara & Adamis, 2016). However, combinations of bevacizumab with immune checkpoint inhibitors in difficult-to-treat tumors such as hepatocellular carcinoma are now standard of care (Finn et al., 2020). It is tempting to speculate that such a combination, perhaps in conjunction with anti-TNFα therapy, will lead to important therapeutic advances.

### Conclusion

The present data points to clearly different inflammatory contexts between localized and metastatic prostate cancer. Primary localized disease demonstrates local inflammation and adaptive immunity, whereas metastases are characterized by immune cold microenvironments and a shift towards resolution of inflammation and tissue repair. Therapies that interfere with these inflammatory networks may offer opportunities for early intervention in monotherapy or in combination with immunotherapies and anti-angiogenic approaches.

### Example 2. Genomics Data Showing that TNFα and its Signaling Pathways Contribute to Immunosuppression

It was found that TNFα and its associated pathways could be targetable in prostate cancer. Using patient derived genomics data it was determined that TNFα and its signaling pathways contribute to immunosuppression and thus reduced immune surveillance in prostate cancer (PCa). Specifically, noncanonical NFKB pathways are activated and AP-1 factors are downregulated.

FIGS. 10A-10J show expression of immune related genes between primary and metastatic sites. Differences in expression of genes associated with immune regulation were observed between primary and metastatic sites. This suggests that site-based differences influence immune signaling.

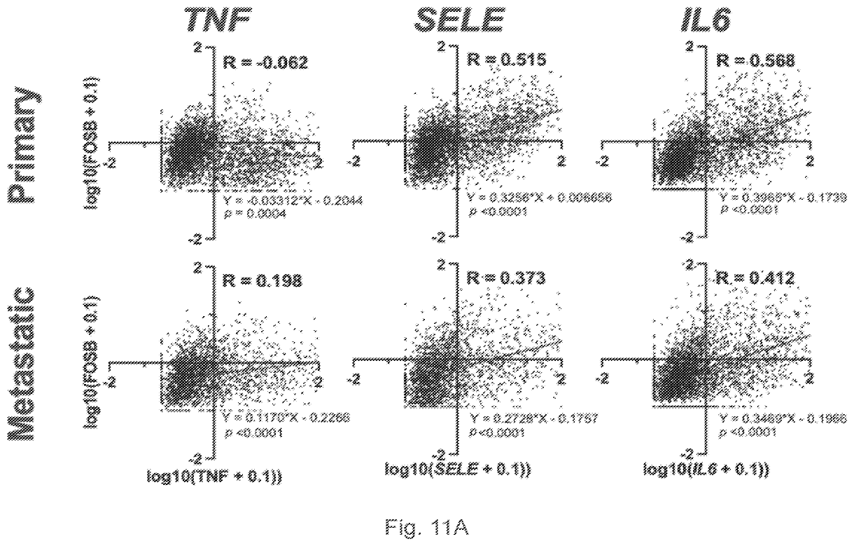

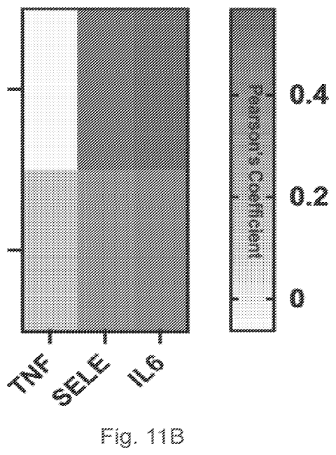

FIG. 11A illustrates a correlation between FOSB and upstream genes a weak relationship between TNFα and FOSB was observed regardless of a primary or metastatic PCa site. SELE and IL6 genes had a stronger association with FOSB in tumors biopsied from the primary as opposed to the metastatic site.

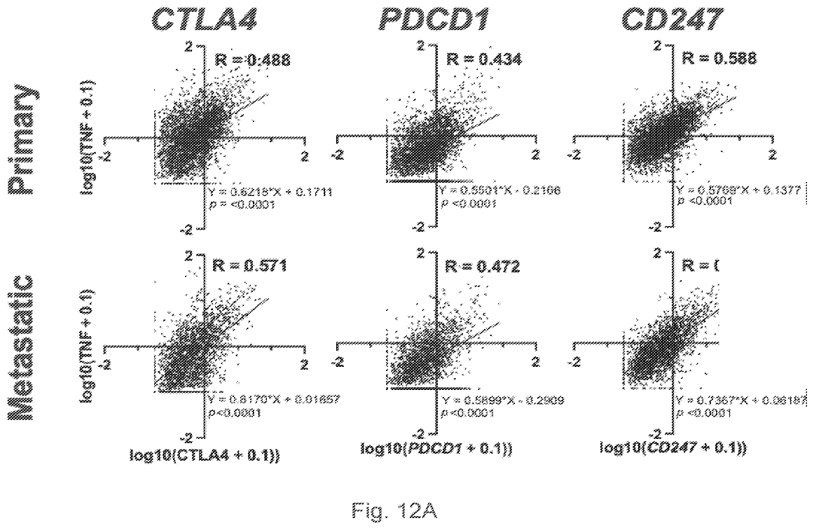

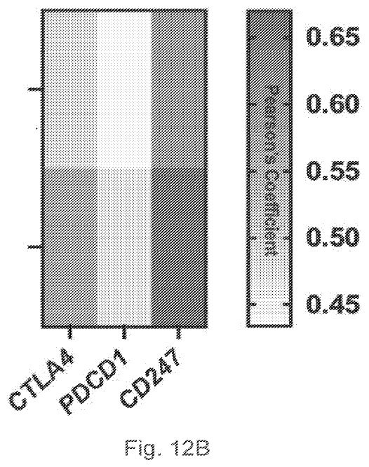

FIG. 12 illustrates a correlation between TNFα and immune checkpoint genes. A strong relationship is observed between immune check point genes and the TNFα ligand across tumors biopsied at either a primary or metastatic site.

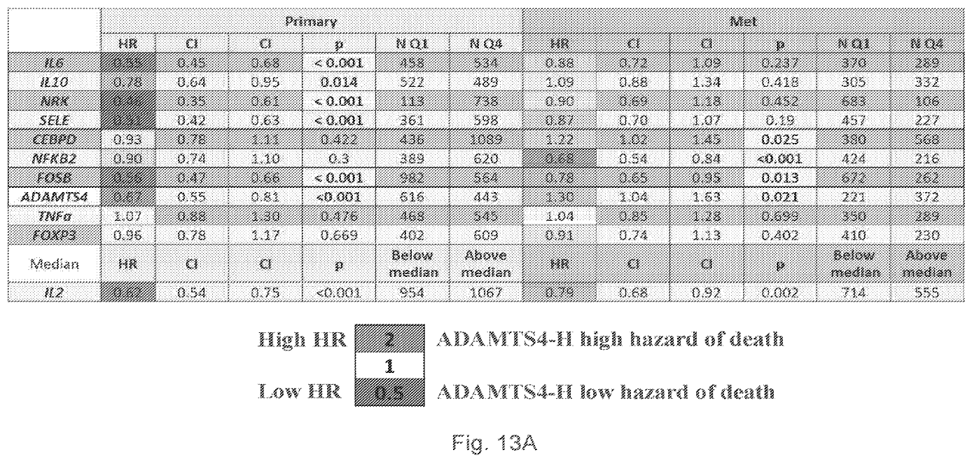

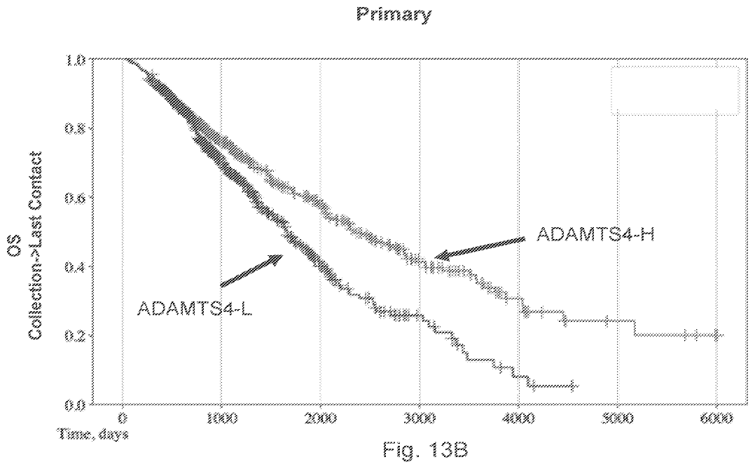

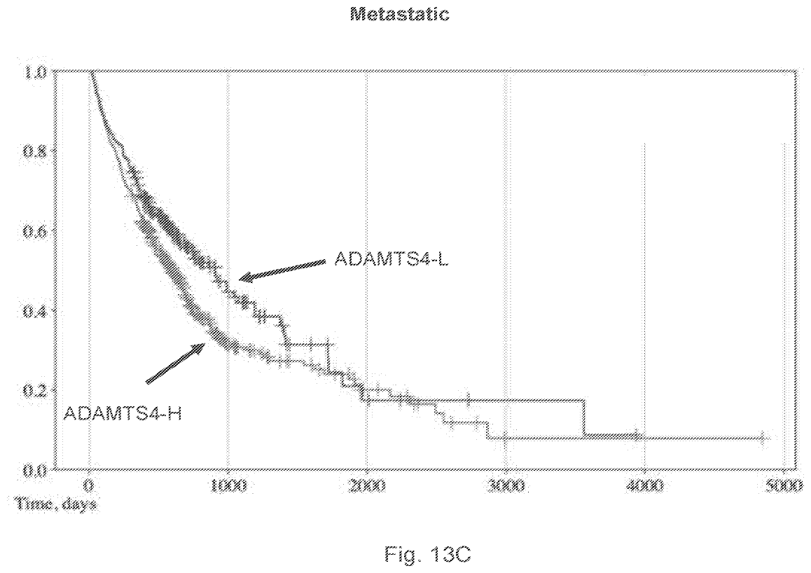

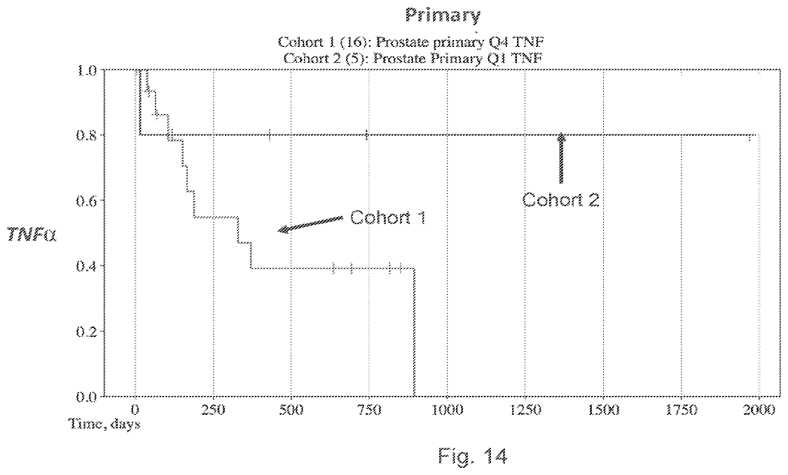

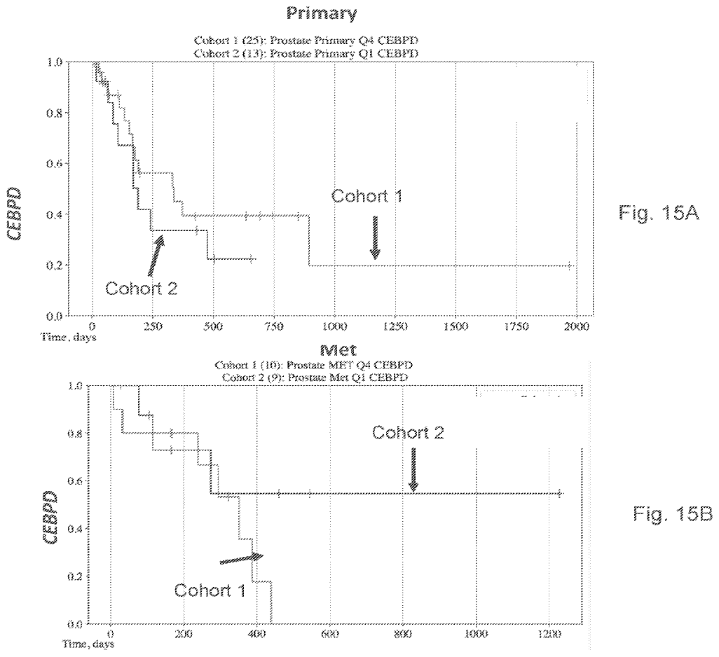

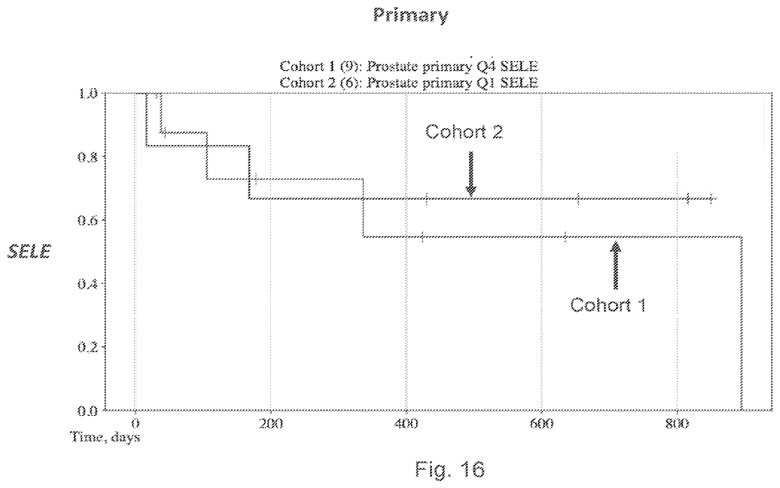

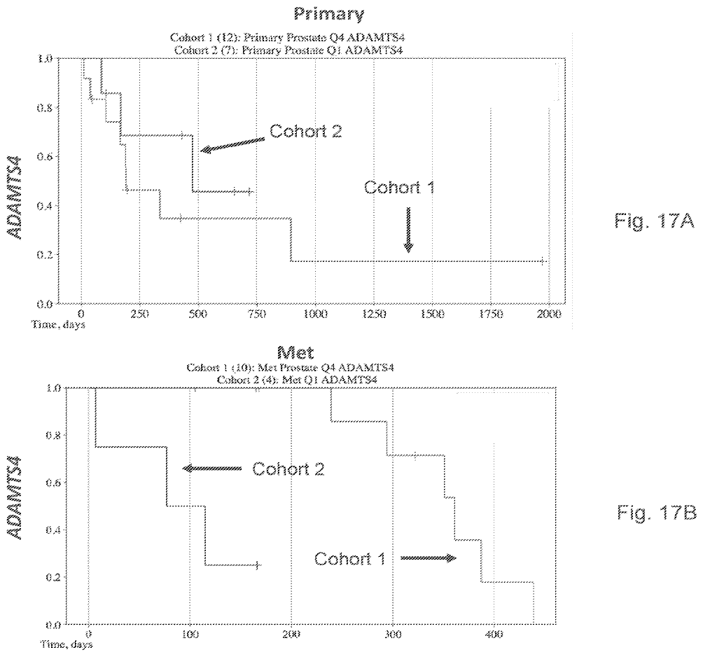

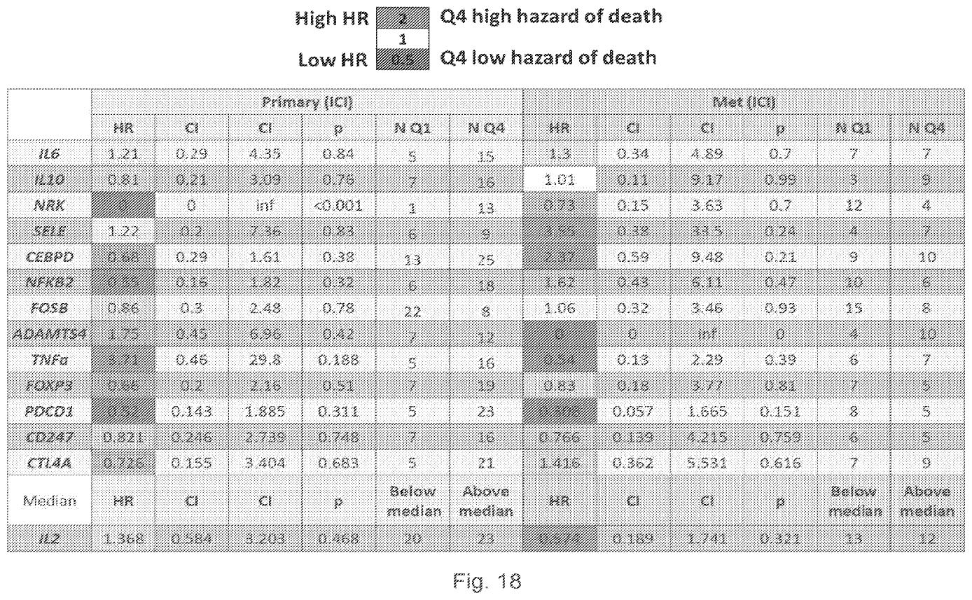

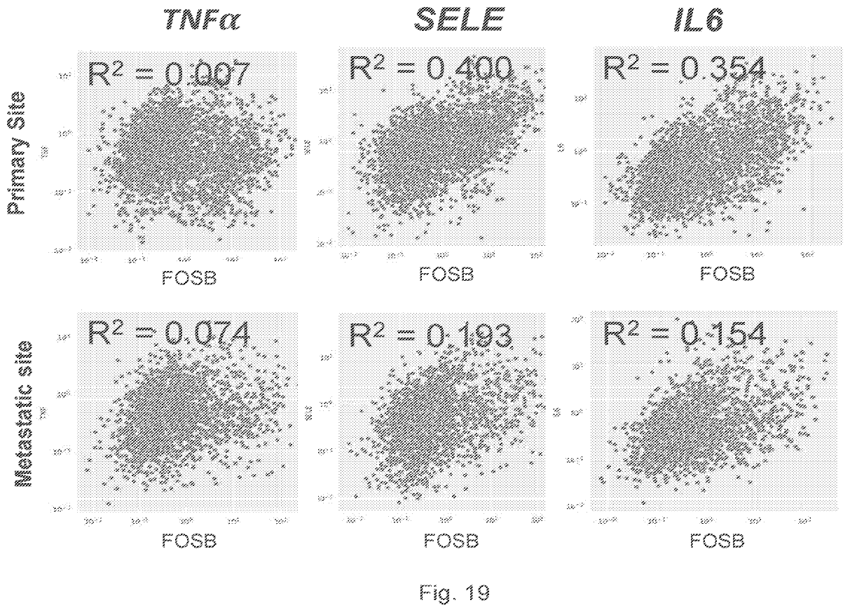

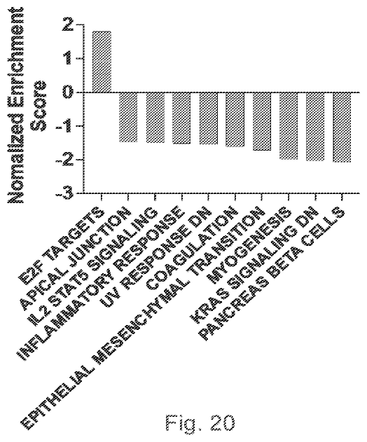

FIGS. 13A-13C show outcomes data (HR=Hazard Risk) in primary vs metastatic sites. High expression of ADAMTS4 is associated with better overall survival (OS) in the primary site but is associated with worse OS in the metastatic site. A similar trend is observed for IL10 and CEBPD2 gene expression.

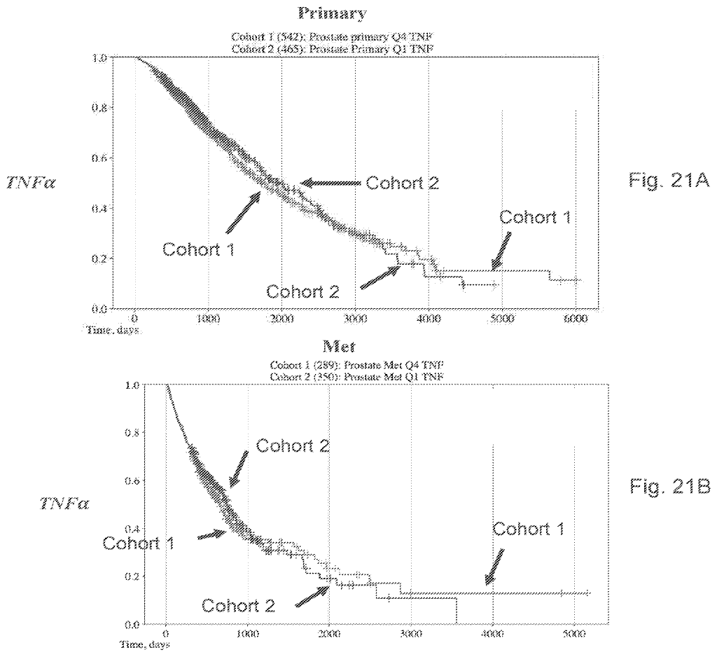

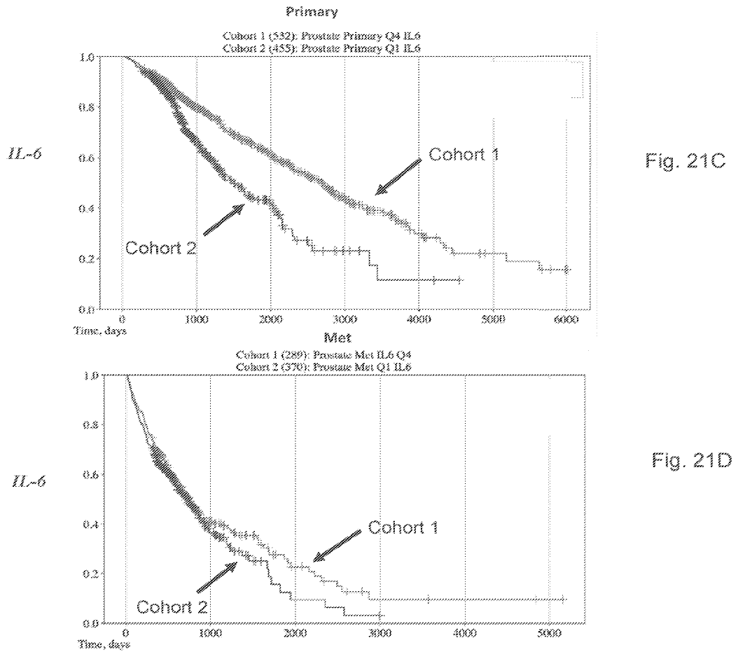

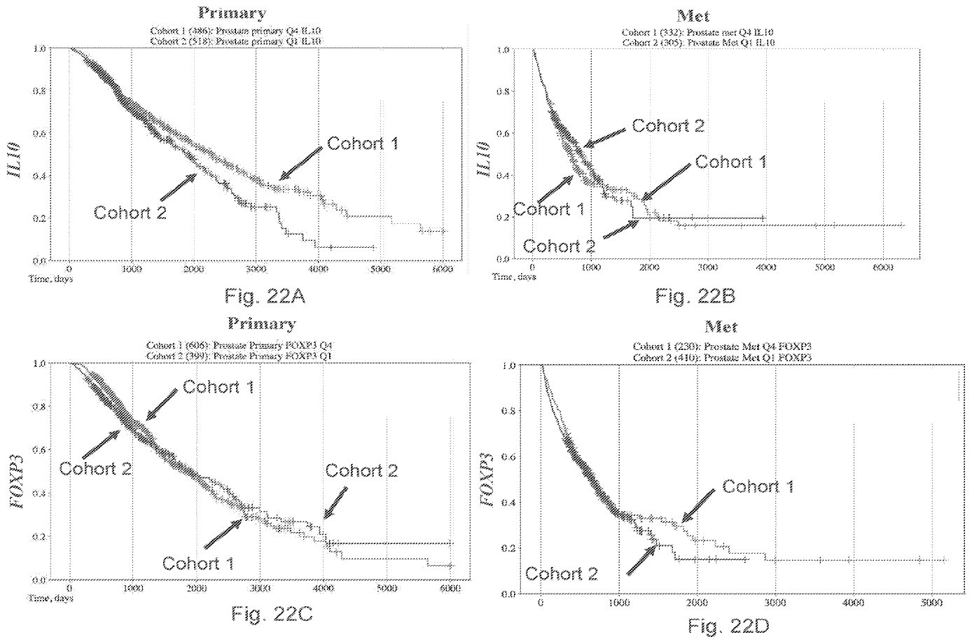

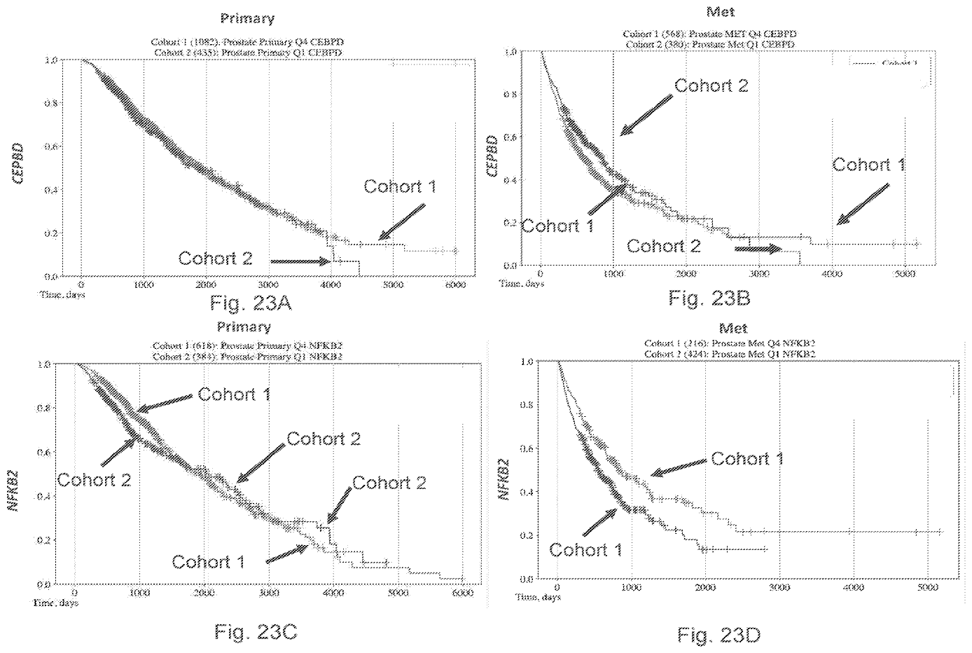

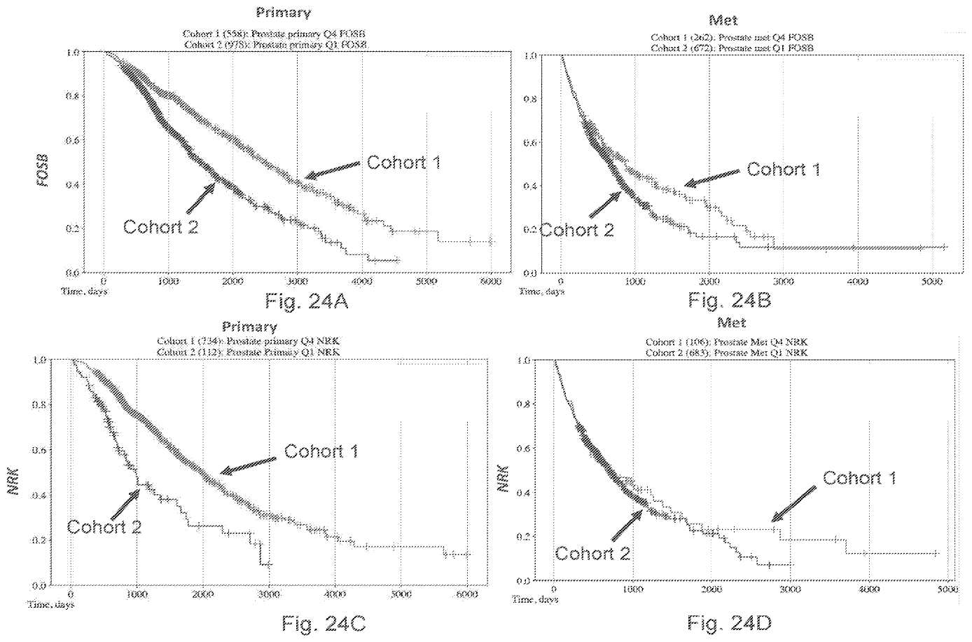

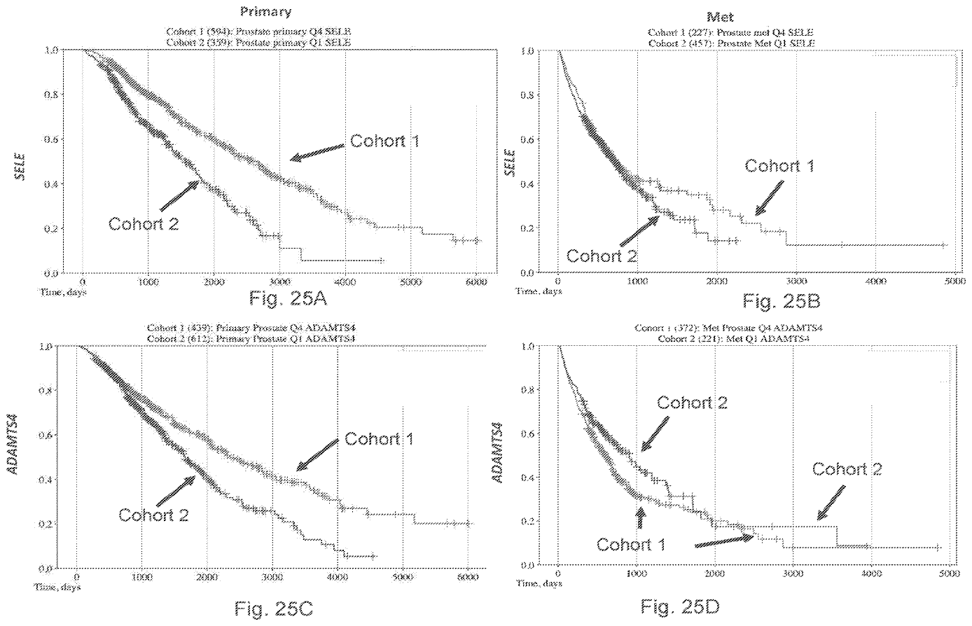

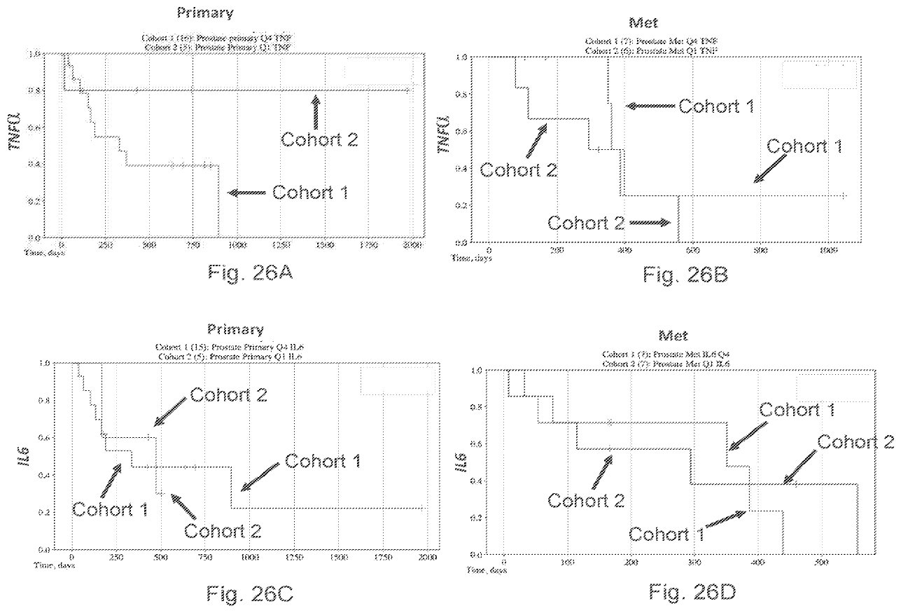

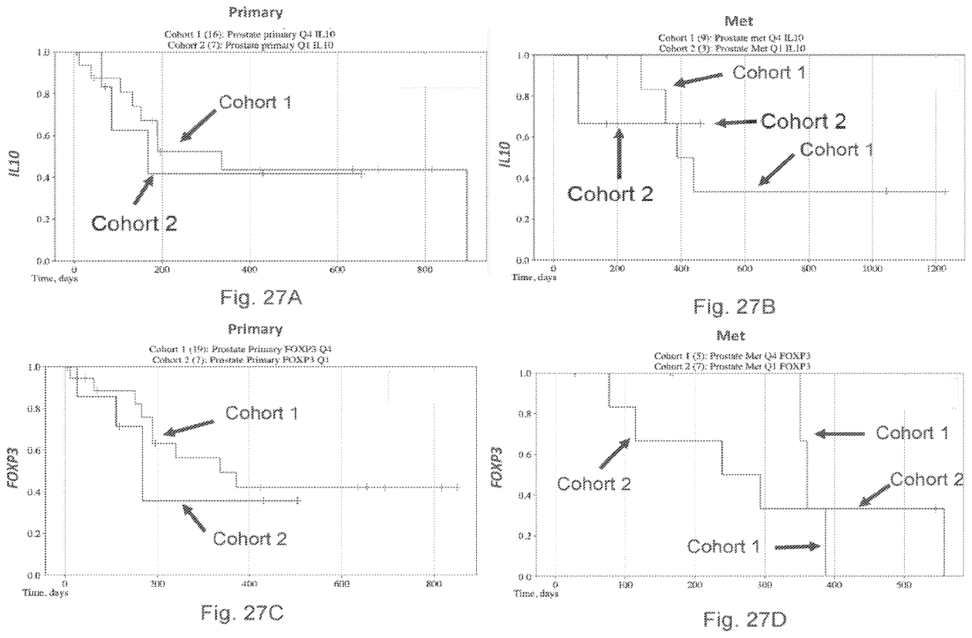

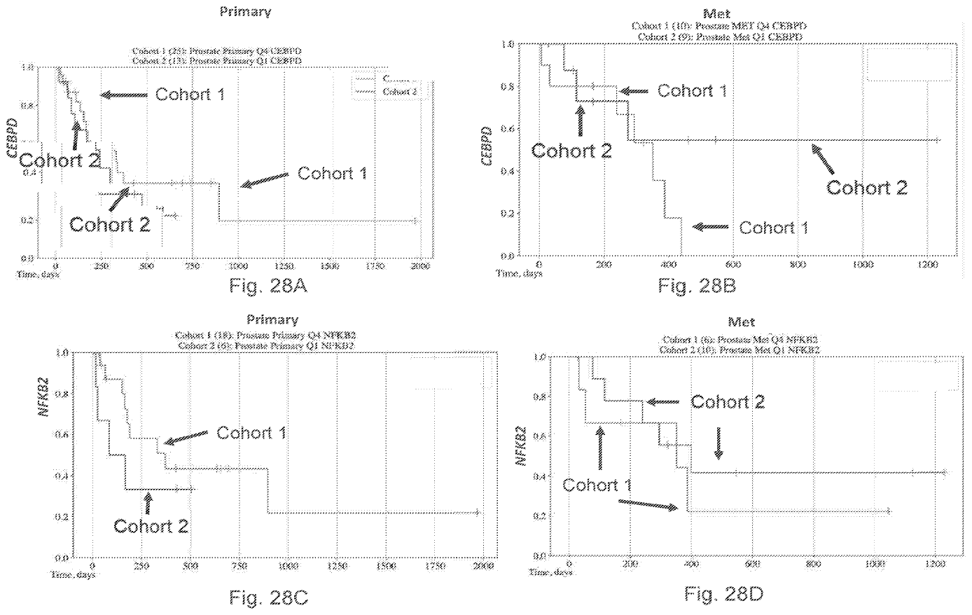

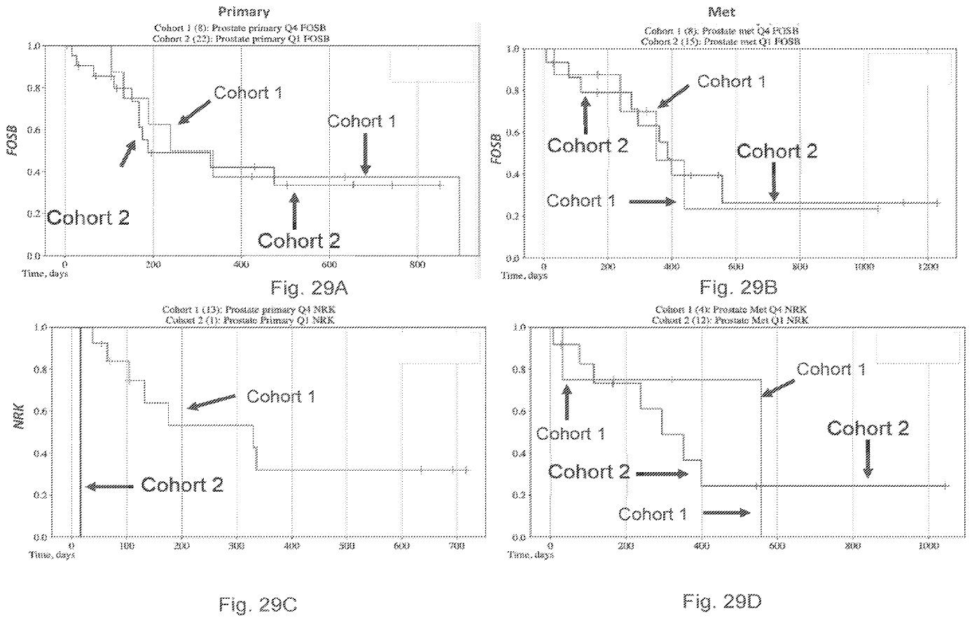

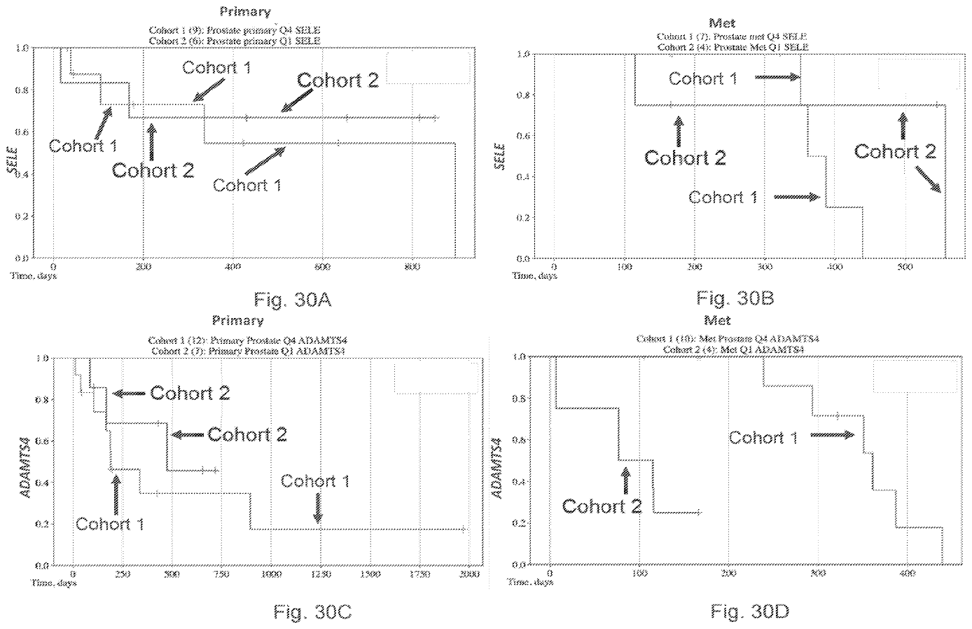

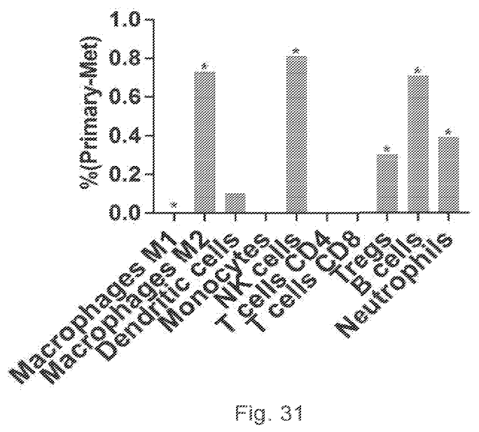

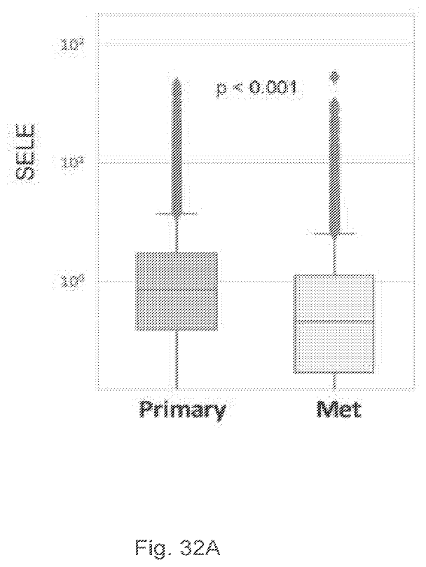

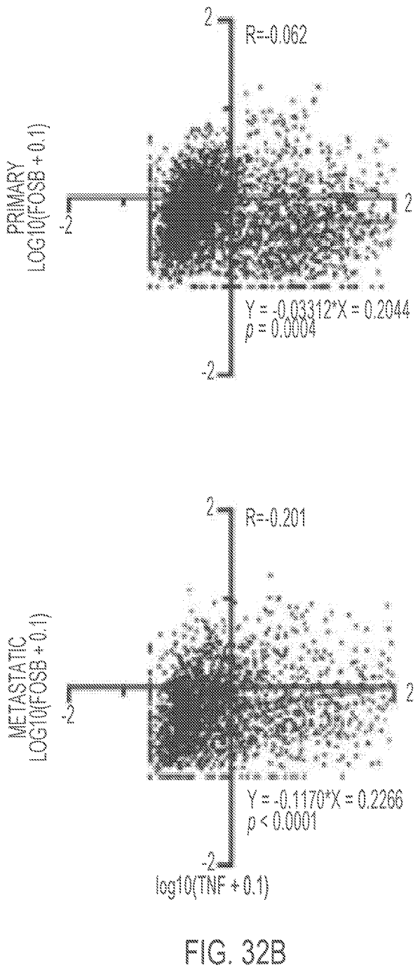

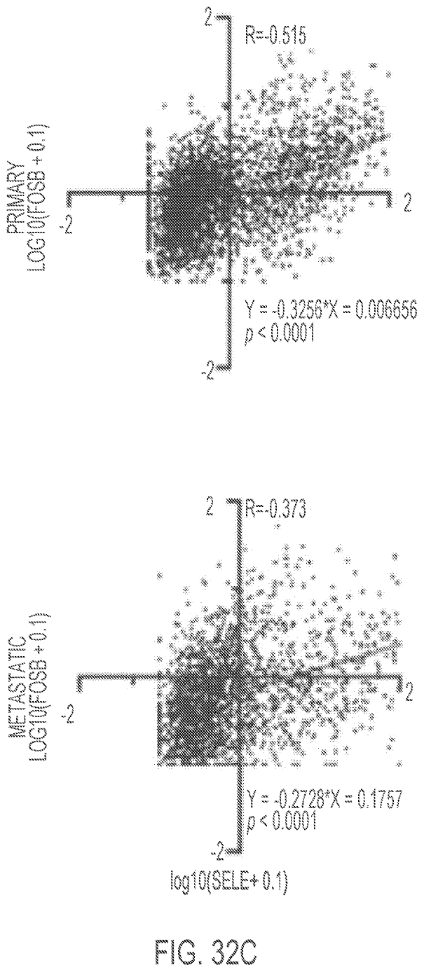

FIG. 21 shows normalized enrichment score comparing Primary vs Metastatic prostate tumors. Positive Normalized enrichment score (NES) suggests enrichment in the primary cohort whereas a negative NES is associated with enrichment in the metastatic cohort. Normalized enrichment score comparing Primary vs Metastatic prostate tumors. all gene sets shown were significantly enriched (p<0.05, FDR<0.1.

TNFα expression directly corelates with PD1, PDL1 and CTLA4 in prostate cancer tissue. This provides a mechanism by which TNFα inhibition may reverse the “cold” prostate cancer into checkpoint (ICI) responsive cancer thus providing a new path for treatment. Patients that expressed low levels of TNFα respond to ICI's treatment based on patient trancriptomics evaluation and clinical outcomes correlations. In addition, ADAMTS4 and NRK which constitute the noncanonical NFKB pathways could be targetable in prostate cancer. Further, a genomics signature of PCa disease progression was identified that includes IL-6, SELE, FOSB, NRK, NFKB2, FOXP3, ARG1, CEBPDP, TNFα, ADAMTS4, PENK, FOSL1, DUSP1, ACTA1, AGT, ATF3, CDK1, CXCL8, SELP, VCAN, TFPI2, NR4A3, and the members of the E2F family of proteins.

### Example 3. Downregulation of E-Selectin and P-Selectin Contributes to Immune Restraining in PCa

The association of TNFα with expression of genes involved in trafficking of circulating immune cells to tumor sites was investigated. Since SELE expression is specific for endothelial cells, it therefore serves as a reliable biomarker of prostate tumor microvasculature. Also, SELE is transcriptionally activated by TNFα in the inflammatory context but this coordinated signaling appears to diverge in the PCa context.

Significantly lower expression levels of SELE in metastatic PCa patient samples were previously observed, but this result was from a relatively small cohort (n=218) (PMID: 36371231). To further validate our finding that SELE expression is downregulated with disease progression, we repeated our analysis in an independent sample cohort (n==5419, localized n==3284, metastatic n=2135). Significant downregulation of SELE was found in metastatic disease (localized mean=0.78 transcripts per million (TPM), metastatic mean=0.41 TPM, p<0.001, FIG. 32A) thus validating the previous result. Similarly, it was validated that SELE downregulation in PCa correlates with significantly lower expression of FOSB and other AP-1 family transcriptional factors. SELE promoter region harbors an AP-1 cis site and therefore transcriptional activity of the locus could be attenuated through lower FOSB expression levels.

Lower expression of SELE, at least in part, can negatively impact the local PCa immune surveillance by preventing tumor-specific immune cell infiltration into the local PCa microenvironment. In turn, this could diminish the availability of T-cells in the tumor thus preventing a vigorous ICIs response. P-Selectin (SELP) expression was found to also be downregulated in PCa disease progression, similarly to that of SELE, thus further contributing to lowering the extravasation capacity of immune cells into the target tissue (localized mean=2.47 TPM, metastatic mean 0.94 TPM, p<0.001). However, the expression levels of functional endothelial integrins, involved in facilitating leucocyte extravasation, such as intercellular adhesion molecule 1 (ICAM1) and vascular cell adhesion molecule 1 (VCAM1). did not significantly differ with disease progression (localized mean=4.62 TPM, metastatic mean=3.48 TPM, and localized mean=6.25 TPM, metastatic mean=4.79 TPM, respectively).

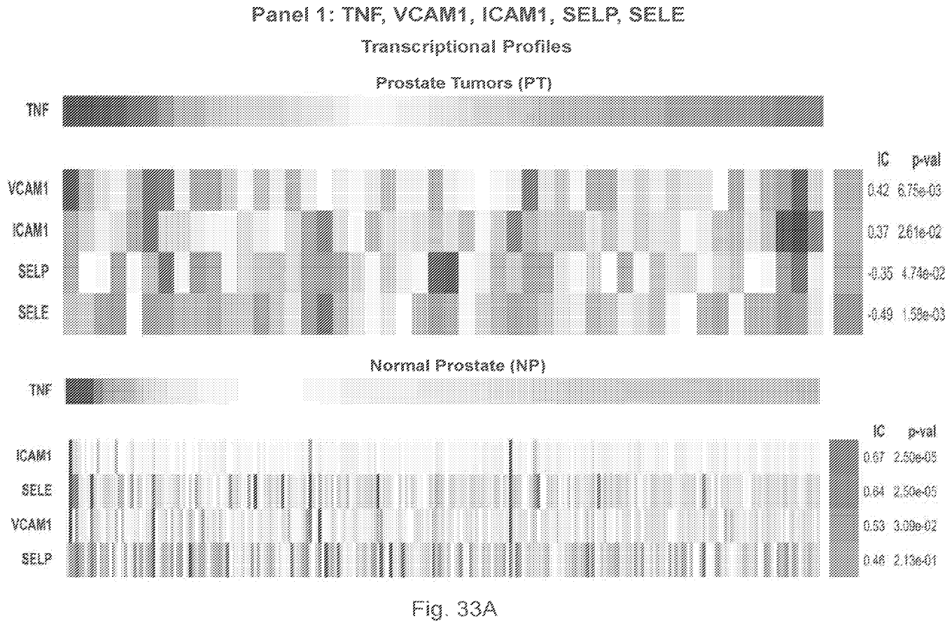

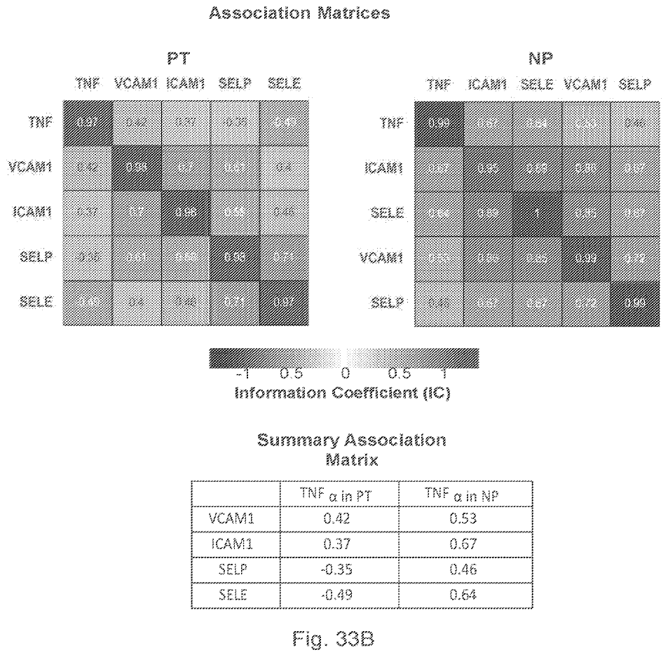

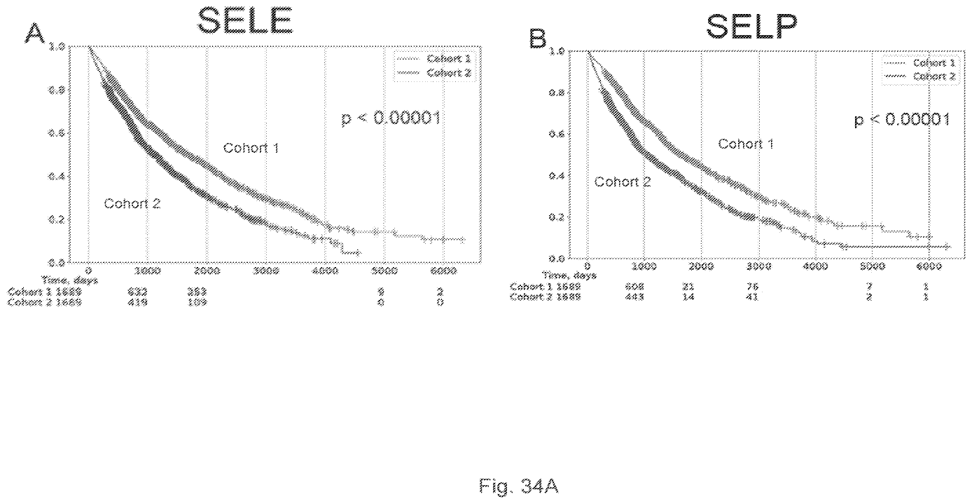

The possibility that PCa local microvasculature “coldness” could be orchestrated by the immunosuppressive PCa microenvironment and in part by “aberrant” TNFα was investigated, where TNFα no longer transcriptionally activates SELE and SELP expression. To examine this possibility, related gene associations were compared in normal prostate versus prostate cancer. Gene expression heatmaps for SELE, SELP as well as the integrins VCAM-1 and ICAM-1 were performed to assess correlation with INFα expression levels. There was a strong divergence in expression levels between these genes in PCa versus normal prostate (NP). TNT expression in normal prostate correlates positively with SELE, SELP, ICAM1 and VCAM1. However, in PCa, TNFα expression correlates negatively with both SELE and SELP while retaining positive correlation with VCAM1 and ICAM1 (FIGS. 33A and 33B).

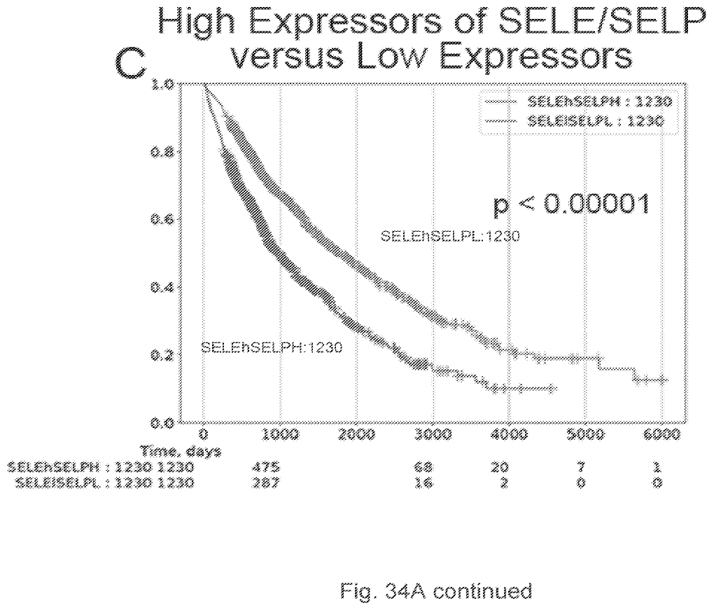

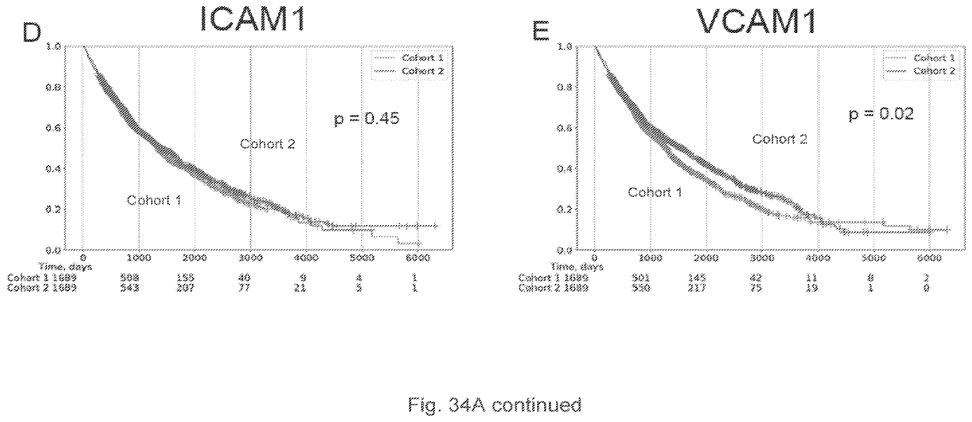

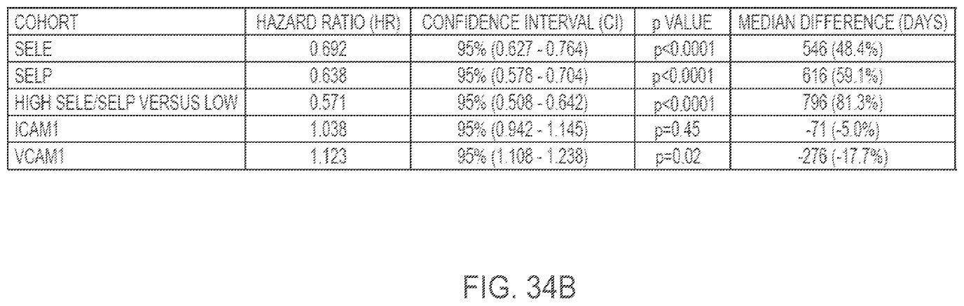

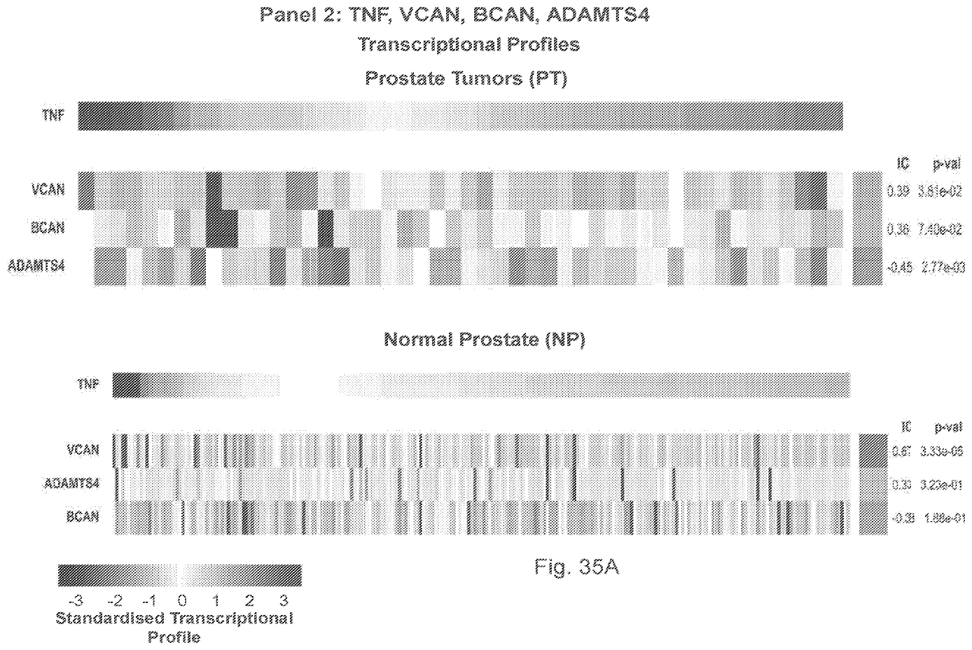

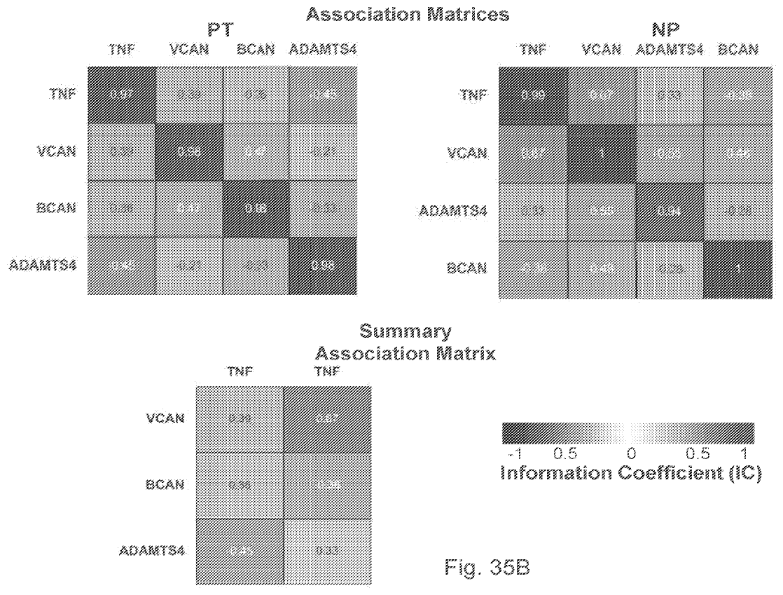

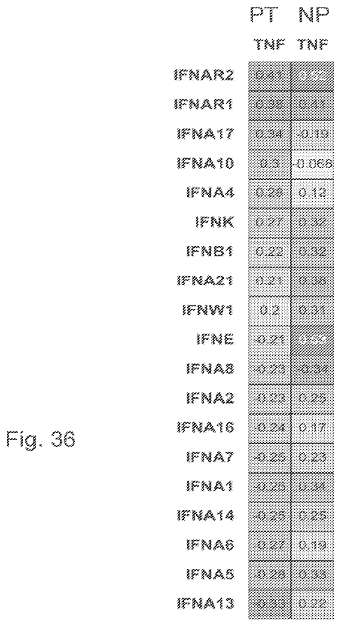

The downregulation of SELE and SELP but not ICAM-1 and VCAM-1 may contribute to PCa immune restraining and tumor “coldness” by favoring the “resident” immune cells while limiting circulating T-cells and other immune cells infiltration into the tumor. This effect would consequently negatively impact immune surveillance and response to ICIs. Thus, higher expression levels of SELE and SELP were predicted but not ICAM1 and VCAM1 and can contribute to improved survival outcomes for patients with PCa and tested our hypothesis by accessing real-world evidence data of PCa patient's survival outcomes. High expression of SELE and SELP was found to be associated with significant survival benefit compared to patients with low SELE or SELP expression levels. (HR=0.692, p<0.00001 and HR=0.641, p<0.0001) (FIG. 34B). However, both VCAM-1 and ICAM-1 expression levels did not correlate with improved survival outcomes (HR=1.123, p=0.02 and HR=1.038, p=0.45). Furthermore, high expression of both SELE and SELP versus low expression of both genes was associated with a synergistic improved survival effect (HR=0.571, p<0.0001) (FIG. 34B).

It has been previously reported that E-Selectin can facilitate the migration of cancer cells to form metastasis in colon, prostate, and other solid tumors. This data was generated in in vitro experimental systems. When SELE function was examined in an in vivo experimental system the result was consistent with its anti-tumor role. It was reported that SELE knockout in mice resulted in tumor growth increase thus implicating SELE in anti-tumor role. It was surprisingly found that SELE and SELP can contribute to tumor “coldness” by remodeling of the local microvasculature to diminish anti-tumor immune cell infiltration into PCa target tissue. These results are based on the analysis of real-world evidence of patients with PCa transcriptomics and survival data. Further research in experimental models is warranted to validate SELE and SELP and their regulatory components as therapeutic targets for PCa immunotherapy.

SELE plays an important role in immune surveillance by facilitating the trafficking of leucocytes into target tissue to mount an appropriate immune response. SELE is solely expressed on endothelial cells and it is transcriptionally induced by pro-inflammatory cytokines such as TNFα, IL-1 and endotoxins. The capacity of circulating immune cells to confer immune response is dependent on their ability to infiltrate target sites. This process involves a coordinated sequence of molecular events initiated by SELE which mediates tethering of circulating leukocytes onto microvascular endothelial cells of the target tissue.

The data provided herein confirm an association between TNFα expression and remodeling of the non-cellular tumor microenvironment with anticipated secondary impacts on immune infiltration. TNFα expression correlates with expression of large extracellular matrix proteoglycans, such as versican, with proven roles in tissue inflammation in cancerous and non-cancerous contexts. By contrast, the expression of versican-degrading enzymes, such as ADAMTS4, is inversely correlated with TNFα expression in prostate cancer tissue. Surprisingly, in normal prostate tissue, ADAMTS4 expression correlates with TNFα expression weakly positively.

### REFERENCES

1. Affairs USDoV. About VHA. https://www.va.gov/health/about vha.asp. Accessed 27 May 2021.
2. Alba et al., JCO Clin Cancer Inform., 09:1005 (2021).
3. Allavena et al., Immunol. Rev., 222:155 (2008).
4. Arlauckas et al., Theranostics, 8:5842 (2018).
5. Barbie et al., Nature, 462:108 (2009).
6. Brennan et al., J. Exp. Clin. Cancer Res., 39:184 (2020).
7. Chen et al., Methods Mol. Biol., 1711:243 (2018).
8. Chen et al., Nat. Cell Biol., 23:87 (2021).
9. Cheng et al., Cell, 184:792 (2021).
10. Chinenov & Kerppola, Oncogene, 20:2438 (2001).
11. Clark et al., Ann. Rheum. Dis., 64:iv70 (2005).
12. Colegio et al., Nature, 513:559 (2014).
13. Consortium G., Nat. Genet., 45:580 (2013).
14. Cross et al., Prostate, 63:269 (2005).
15. Deeble et al., Mol. Cell Biol., 21:8471 (2001).
16. Dimitroff et al., Cancer Res., 65:5750 (2005).
17. Eferl & Wagner, Nat. Rev. Cancer, 3:859 (2003).
18. Ferrara & Adamis, Nat. Rev. Drug Discov., 15:385 (2016).
19. Finn et al., N. Engl. J. Med., 382:1894 (2020).
20. Fizazi et al., Eur. J. Cancer, 48:85 (2012).
21. Harwood et al., J. Biol. Chem., 275:10023 (2000).
22. Hwang et al., J. Transl. Med., 18:443 (2020).
23. Jochum et al., Oncogene, 20:2401 (2001).
24. Kim et al., Arthritis Rheum., 56:1076 (2007).
25. Kim et al., Nat. Biotechnol., 34:539 (2016).
26. Kim et al., Nature, 457:102 (2009).
27. Kollias et al., Curr. Dir. Autoimmun., 5:30 (2002).
28. Liberzon et al., Bioinformatics, 27:1739 (2011).
29. Liberzon et al., Cell Syst., 1:417 (2015).
30. Liu et al., Int. J. Gen. Med., 15:2171 (2022).
31. Liu et al., J. Urol., 189:2262 (2013).
32. Martinez et al., J. Immunol., 177:7303 (2006).
33. Michalaki et al., Br. J. Cancer, 90:2312 (2004).
34. Mulder et al., Immunity, 54:1883 (2021).
35. News R U. Prostate cancer incidence higher in men with autoimmune disease. Haymarket Media, Inc. Accessed 22 Sep. 2022.
36. Nguyen et al., BJU Int., 113:986 (2014).
37. Ozanne et al., Oncogene, 26:1 (2007).
38. Papadas & Asimakopoulos, Adv. Exp. Med. Biol., 1272:55 (2020).
39. Papadas et al., Cell Rep., 40:111201 (2022).
40. R: A language and environment for statistical computing. Version 4.0.1. R Foundation for Statistical Computing (2020).
41. Reschke & Gajewski, 7:eabg6509 (2022).
42. Robbins et al., Robbins pathologic basis of disease. Philadelphia: W B Saunders (1999).
43. Shariat et al., Urology, 58:1008 (2001).
44. Shaulian & Karin, Oncogene, 20:2390 (2001).
45. Smith et al., Cytokine Growth Factor Rev., 12:33 (2001).
46. Spranger et al., Cancer Cell, 31:711 (2017).
47. Steen et al., Methods Mol. Biol., 2117:135 (2020).
48. Subramanian et al., Proc. Natl. Acad. Sci. USA, 102:15545 (2005).
49. Tang et al., Cell Rep., 13:2851 (2015).
50. Thomsen et al., Cell Death Differ., 22:574 (2015).
51. Twillie et al., Urology, 45:542 (1995).
52. Vadevoo et al., Proc. Natl. Acad. Sci. USA (2021).
53. Vartanian et al., Mol. Cancer Res., 9:115 (2011).
54. Vickman et al., Nat. Commun., 13:2133 (2022).
55. Ye et al., Ann. Transl. Med., 8:1647 (2020).
56. Yu et al., Cancer Immunol. Res., 3:1175 (2015).
57. Zheng et al., Science, 374:abe6474 (2021).
58. Islam S, Watanabe H. Versican: A Dynamic Regulator of the Extracellular Matrix. J Histochem Cytochem. 2020; 68(11):763-75.
59. Papadas A, Asimakopoulos F. Versican in the Tumor Microenvironment. Adv Exp Med Biol. 2020; 1272:55-72.
60. Wight T N, Kang I, Merrilees M J. Versican and the control of inflammation. Matrix Biol. 2014; 35:152-61.
61. Hope C, Ollar S J, Heninger E, Hebron E, Jensen J L, Kim J, et al. TPL2 kinase regulates the inflammatory milieu of the myeloma niche. Blood. 2014; 123(21):3305-15.
62. Kim S, Takahashi H, Lin W W, Descargues P, Grivennikov S, Kim Y, et al. Carcinoma-produced factors activate myeloid cells through TLR2 to stimulate metastasis. Nature. 2009; 457(7225):102-6.
63. Tang M, Diao J, Gu H, Khatri I, Zhao J, Cattral M S. Toll-like Receptor 2 Activation Promotes Tumor Dendritic Cell Dysfunction by Regulating IL-6 and IL-10 Receptor Signaling. Cell Rep. 2015; 13(12):2851-64.
64. Hope C, Foulcer S, Jagodinsky J, Chen S X, Jensen J L, Patel S, et al. Immunoregulatory roles of versican proteolysis in the myeloma microenvironment. Blood. 2016; 128(5):680-5.
65. Papadas A, Deb G, Cicala A, Officer A, Hope C, Pagenkopf A, et al. Stromal remodeling regulates dendritic cell abundance and activity in the tumor microenvironment. Cell Rep. 2022; 40(7):111201.
66. Hope C, Emmerich P B, Papadas A, Pagenkopf A, Matkowskyj K A, Van De Hey D R, et al. Versican-Derived Matrikines Regulate Batf3-Dendritic Cell Differentiation and Promote T Cell Infiltration in Colorectal Cancer. J Immunol. 2017; 199(5):1933-41.
67. Cross N A, Chandrasekharan S, Jokonya N, Fowles A, Hamdy F C, Buttle D J, et al. The expression and regulation of ADAMTS-1, -4, -5, -9, and -15, and TIMP-3 by TGFbeta1 in prostate cells: relevance to the accumulation of versican. Prostate. 2005; 63(3):269-75.
68. Flavell R A, Sanjabi S, Wrzesinski S H, Licona-Limon P. The polarization of immune cells in the tumour environment by TGFbeta. Nat Rev Immunol. 2010; 10(8):554-67.
69. Wight T N. Versican: a versatile extracellular matrix proteoglycan in cell biology. Curr Opin Cell Biol. 2002; 14(5):617-23.

All patents and publications referenced or mentioned herein are indicative of the levels of skill of those skilled in the art to which the invention pertains, and each such referenced patent or publication is hereby specifically incorporated by reference to the same extent as if it had been incorporated by reference in its entirety individually or set forth herein in its entirety. Applicants reserve the right to physically incorporate into this specification any and all materials and information from any such cited patents or publications.

The following statements are intended to describe and summarize various embodiments of the invention according to the foregoing description in the specification.

### STATEMENTS

1. A method to predict disease progression or a risk of disease progression in a mammal with prostate cancer, comprising:
   (a) assaying a biological sample comprising prostate tissue from a subject for expression of genes comprising IL-6, SELE, FOSB, NRK, NFKB2, FOXP3, ARG1, CEBPDP, TNFα, ADAMTS4, PENK, FOSL1, DUSP1, ACTA1, AGT, ATF3, CDK1, CXCL8, SELP, VCAN, TFPI2, NR4A3, or combinations thereof, to determine one or more expression levels for the IL-6, SELE, FOSB, NRK, NFKB2, FOXP3, ARG1, CEBPDP, TNFα, ADAMTS4, PENK, FOSL1, DUSP1, ACTA1, AGT, ATF3, CDK1, CXCL8, SELP, VCAN, TFPI2, or NR4A3 genes;
   (b) comparing the determined expression levels with one or more reference values to identify any altered expression levels in the subject's biological sample, wherein altered expression levels of the IL-6, SELE, FOSB, NRK, NFKB2, FOXP3, ARG1, CEBPDP, TNFα, ADAMTS4, PENK, FOSL1, DUSP1, ACTA1, AGT, ATF3, CDK1, CXCL8, SELP, VCAN, TFPI2, NR4A3, or combinations thereof in the biological sample relative to the reference value indicates that the subject has PCa with a high risk of developing metastasized PCa; and
   (c) administering one or more TNFα antagonists, SELE agonists, or immune checkpoint inhibitors (ICI) to a subject determined to have the high risk of developing metastasized PCa.
2. The statement of claim 1, wherein the expression of genes for SELE, FOSB, NRK, ADAMTS4, and NR4A3 are assayed.
3. The statement of claim 2, wherein:
   the gene for SELE has a nucleic acid sequence of SEQ ID NO: 4,
   the gene for FOSB has a nucleic acid sequence of SEQ ID NO: 6,
   the gene for NRK has a nucleic acid sequence of SEQ ID NO: 8,
   the gene for ADAMTS4 has a nucleic acid sequence of SEQ ID NO: 14, and
   the gene for NR4A3 has a nucleic acid sequence of SEQ ID NO: 18.
4. The statement of claim 1 wherein the mammal is a human.
5. The statement of claim 1, 2, or 3 wherein expression of three or more of IL-6, SELE, FOSB, NRK, NFKB2, FOXP3, ARG1, CEBPDP, TNFα, ADAMTS4, PENK, FOSL1, DUSP1, ACTA1, AGT, ATF3, CDK1, CXCL8, SELP, VCAN, TFPI2, or NR4A3 genes are detected.
6. The statement of claim 1, 2, or 3 wherein five or more of IL-6, SELE, FOSB, NRK, NFKB2, FOXP3, ARG1, CEBPDP, TNFα, ADAMTS4, PENK, FOSL1, DUSP1, ACTA1, AGT, ATF3, CDK1, CXCL8, SELP, VCAN, TFPI2, or NR4A3 are detected.
7. The statement of any one of claim 1 to 6 wherein RNA expression is detected.
9. The statement of any one of claim 1 to 6 wherein protein expression is detected.
10. The statement of claim 9, wherein:
   the protein for IL6 has an amino acid sequence with at least 95% sequence identity to SEQ ID NO: 1;
   the protein for SELE has an amino acid sequence with at least 95% sequence identity to SEQ ID NO: 3;
   the protein for FOSB an amino acid sequence with at least 95% sequence identity to SEQ ID NO: 5;
   the protein for NRK has an amino acid sequence with at least 95% sequence identity to SEQ ID NO: 9;
   the protein for TNFα has an amino acid sequence with at least 95% sequence identity to SEQ ID NO: 11;
   the protein for ADAMTS4 has an amino acid sequence with at least 95% sequence identity to SEQ ID NO: 13;
   the protein for SELP has an amino acid sequence with at least 95% sequence identity to SEQ ID NO: 15; and
   the protein for NR4A3 has an amino acid sequence with at least 95% sequence identity to SEQ ID NO: 17.
11. The statement of claim 1, wherein the one or more TNFα antagonists comprise infliximab, adalimumab, etanercept, golimumab, certolizumab, adalimumab, certolizumab, erelzi, golimumab, and etanercept.
12. The statement of claim 1, wherein the one or more ICIs comprise tecentriq, libtayo, keytruda, opdivo, and yervoy.
13. The statement of claim 1, further comprising administering a chemotherapeutic agent.
14. The statement of claim 13, wherein the chemotherapeutic agent is avastin.
15. A method of inhibiting or treating disease progression in a mammal with prostate cancer, comprising: administering to the mammal an effective amount of a TNFα inhibitor (antagonist), a SELE agonist, an immune checkpoint inhibitors (ICI), or an anti-angiogenic agent, or a combination thereof, wherein the mammal has an expression profile of one or more of IL-6, SELE, FOSB, NRK, NFKB2, FOXP3, ARG1, CEBPDP, TNFα, ADAMTS4, PENK, FOSL1, DUSP1, ACTA1, AGT, ATF3, CDK1, CXCL8, SELP, VCAN, TFPI2, NR4A3, or any combination thereof, that is indicative of increased risk of disease progression.
16. The statement of claim 15 wherein the mammal is a human.
17. The statement of claim 15 or 16 wherein expression of three or more of IL-6, SELE, FOSB, NRK, NFKB2, FOXP3, ARG1, CEBPDP, TNFα, ADAMTS4, PENK, FOSL1, DUSP1, ACTA1, AGT, ATF3, CDK1, CXCL8, SELP, VCAN, TFPI2, or NR4A3 genes are detected.
18. The statement of any one of claim 15 to 17 wherein RNA expression is detected.
19. The statement of claim 15, wherein the expression of genes for SELE, FOSB, NRK, ADAMTS4, and NR4A3 are assayed.
20. A kit comprising at least one isolated probe that hybridizes to RNA for one or more of IL-6, SELE, FOSB, NRK, NFKB2, FOXP3, ARG1, CEBPDP, TNFα, ADAMTS4, PENK, FOSL1, DUSP1, ACTA1, AGT, ATF3, CDK1, CXCL8, SELP, VCAN, TFPI2, NR4A3, or any combination thereof, that is optionally bound to a solid support or at least one primer having a nucleotide sequence for detecting one or more of IL-6, SELE, FOSB, NRK, NFKB2, FOXP3, ARG1, CEBPDP, TNFα, ADAMTS4, PENK, FOSL1, DUSP1, ACTA1, AGT, ATF3, CDK1, CXCL8, SELP, VCAN, TFPI2, NR4A3, or any combination thereof.
21. The statement of claim 20 further comprising instructions for using the at least one probe or at least one primer in the method of claim 1.
22. The statement of claim 20 wherein the solid support is selected from the group consisting of a bead, plate, membrane, array, or chip.

The specific methods, devices and compositions described herein are representative of preferred embodiments and are exemplary and not intended as limitations on the scope of the invention. Other objects, aspects, and embodiments will occur to those skilled in the art upon consideration of this specification, and are encompassed within the spirit of the invention as defined by the scope of the claims. It will be readily apparent to one skilled in the art that varying substitutions and modifications can be made to the invention disclosed herein without departing from the scope and spirit of the invention.

The invention illustratively described herein suitably can be practiced in the absence of any element or elements, or limitation or limitations, which is not specifically disclosed herein as essential. The methods and processes illustratively described herein suitably can be practiced in differing orders of steps, and the methods and processes are not necessarily restricted to the orders of steps indicated herein or in the claims.

Under no circumstances can the patent be interpreted to be limited to the specific examples or embodiments or methods specifically disclosed herein. Under no circumstances can the patent be interpreted to be limited by any statement made by any Examiner or any other official or employee of the Patent and Trademark Office unless such statement is specifically and without qualification or reservation expressly adopted in a responsive writing by Applicants.

The terms and expressions that have been employed are used as terms of description and not of limitation, and there is no intent in the use of such terms and expressions to exclude any equivalent of the features shown and described or portions thereof, but it is recognized that various modifications are possible within the scope of the invention as claimed. Thus, it will be understood that although the present invention has been specifically disclosed by preferred embodiments and optional features, modification and variation of the concepts herein disclosed can be resorted to by those skilled in the art, and that such modifications and variations are considered to be within the scope of this invention as defined by the appended claims and statements of the invention.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein. In addition, where features or aspects of the invention are described in terms of Markush groups, those skilled in the art will recognize that the invention is also thereby described in terms of any individual member or subgroup of members of the Markush group.

1. A method to predict disease progression or a risk of disease progression in a mammal with prostate cancer, comprising:
(a) assaying a biological sample comprising prostate tissue from a subject for expression of genes comprising IL-6, SELE, FOSB, NRK, NFKB2, FOXP3, ARG1, CEBPDP, TNFα, ADAMTS4, PENK, FOSL1, DUSP1, ACTA1, AT, ATF3, CDK1, CXCL8, SE-LP, WAN, TFPI2, NR4A3, or combinations thereof, to determine one or more expression levels for the IL-6, SELE, FOSB, NRK, NFKB2, FOXP3, ARG1, CEBPDP, TNFα, ADAMTS4, PENK, FOSL1, DUSP1, ACTA1, AGT, ATF3, CDK1, CXCL8, SELP, VCAN, TFPI2, or NR4A3 genes; and
(b) comparing the determined expression levels with one or more reference values to identify any altered expression levels in the subject's biological sample, wherein altered expression levels of the IL-6, SELE, FOSB, NRK, NFKB2, FOXP3, ARG1, CEBPDP, TNPα, ADAMTS4, PENK, FOSL1, DUSP1, ACTA1, AGT, ATF3, CDK1, CXCL8, SELP, VCAN, TFPI2, NR4A3, or combinations thereof in the biological sample relative to the reference value indicates that the subject has PCa with a high risk of developing metastasized PCa.

2. The method of claim 1, wherein the expression of genes for SELE, FOSB, NRK, ADAMTS4, and NR4A3 are assayed.

3. The method of claim 2, wherein:
the gene for SELE has a nucleic acid sequence of SEQ ID NO: 4,
the gene for FOSB has a nucleic acid sequence of SEQ ID NO: 6,
the gene for NRK has a nucleic acid sequence of SEQ ID NO: 8,
the gene for ADAMTS4 has a nucleic acid sequence of SEQ ID NO: 14, and
the gene for NR4A3 has a nucleic acid sequence of SEQ ID NO: 18.

4. The method of claim 1 wherein the mammal is a human.

5. The method of claim 1 wherein expression of three or more of IL-6, SELE, FOSB, NRK, NFKB2, FOXP3, ARG1, CEBPDP, TNFα, ADAMTS4, PENK, FOSL1, DUSP1, ACTA1, AGT, ATF3, CDK1, CXCL8, SELP, VCAN, TFPI2, or NR4A3 genes are detected.

6. The method of claim 1 wherein five or more of IL-6, SELE, FOSB, NRK, NFKB2, FOXP3, ARG, CEBPDP, TNFα, ADAMTS4, PENK, FOSL1, DUSP1, ACTA1, ACT, ATF3, CDK1, CXCL8, SELP, VCAN, TFPI2, or NR4A3 are detected.

7. The method of claim 1 wherein RNA expression is detected.

8. The method of claim 1, further comprising administering one or more TNF, antagonists, SELE agonists, or immune checkpoint inhibitors (ICI) to a subject determined to have the high risk of developing metastasized PCa.

9. The method of claim 1 wherein protein expression is detected.

10. The method of claim 9, wherein:
the protein for IL6 has an amino acid sequence with at least 95% sequence identity to SEQ ID NO: 1;
the protein for SELE has an amino acid sequence with at least 95% sequence identity to SEQ ID NO: 3,
the protein for FOSB an amino acid sequence with at least 95% sequence identity to SEQ ID NO: 5;
the protein for NRK has an amino acid sequence with at least 95% sequence identity to SEO ID NO: 9,
the protein for TNFα has an amino acid sequence with at least 95% sequence identity to SEQ ID NO: 11;
the protein for ADAMTS4 has an amino acid sequence with at least 95% sequence identity to SEQ ID NO: 13;
the protein for SELP has an amino acid sequence with at least 95% sequence identity to SEQ ID NO: 15; and
the protein for NR4A3 has an amino acid sequence with at least 95% sequence identity to SEQ ID NO: 17.

11. The method of claim 8, wherein the one or more TNFα antagonists comprise infliximab, adalimumab, etanercept, golimumab, certolizumab, adalimumab, certolizumab, erelzi, golimumab, and etanercept.

12. The method of claim 8, wherein the one or more ICIs comprise tecentriq, libtayo, keytruda, opdivo, and yervoy.

13. The method of claim 8, further comprising administering a chemotherapeutic ag ent.

14. The method of claim 13, wherein the chemotherapeutic agent is avastin.

15. A method of inhibiting or treating disease progression in a mammal with prostate cancer, comprising: administering to the mammal an effective amount of a TNFα inhibitor (antagonist), a SELF agonist, an immune checkpoint inhibitors (ICI), or an anti-angiogenic agent, or a combination thereof, wherein the mammal has an expression profile of one or more of IL-6, SELE, FOSB, NRK, NFKB2, FOXP3, ARG1, CEBPDP, TNFα, ADAMTS4, PENK, FOSL1, DUSP1, ACTA1, AGT, ATF3, CDK1, CXCL8, SELP, WAN, TFPI2, NR4A3, or any combination thereof, that is indicative of increased risk of disease progression.

16. The method of claim 15 wherein the mammal is a human.

17. The method of claim 15 wherein expression of three or more of IL-6, SELE, FOSB, NRK, NFKB2, FOXP3, ARG1, CEBPDP, TNFα, ADAMTS4, PENK, FOSL1, DUSP1, ACTA1, AGT, ATF3, CDK1, CXCL8, SELP, VCAN, TFPI2, or NR4A3 genes are detected.

18. The method of claim 15 wherein RNA expression is detected.

19. The method of claim 15, wherein the expression of genes for SELE, FOSB, NKK, ADAMTS4, and NR4A3 are assayed.

20. A kit comprising at least one isolated probe that hybridizes to RNA for one or more of IL-6, SELE, FOSB, NRK, NFKB2, FOXP3, ARG1, CEBPDP, TNFα, ADAMTS4, PENK, FOSL1, DUSP1, ACTA1, AGT, ATF3, CDK1, CXCL8, SELP, VCAN, TFPI2, NR4A3, or any combination thereof, that is optionally bound to a solid support or at least one primer having a nucleotide sequence for detecting one or more of IL-6, SELE, FOSB, NRK, NFKB2, FOXP3, ARG1, CEBPDP, TNFα, ADAMTS4, PENK, FOSL1, DUSP1, ACTA1, AGT, ATF3, CDK1, CXCL8, SELP, VCAN, TFPI2, NR4A3, or any combination thereof.

21. The kit of claim 20 further comprising instructions for using the at least one probe or at least one primer in the method of claim 1.

22. The kit of claim 20 wherein the solid support is selected from the group consisting of a bead, plate, membrane, array, or chip.


SEQUENCE LISTING (18 sequences)

SEQ ID NO: 1 - protein, 212 aa, Homo sapiens
MNSFSTSAFGPVAFSLGLLLVLPAAFPAPVPPGEDSKDVAAPHRQPLTSSERIDKQIRYILDGISALRKETCNKSNMCESSKEALAENNLNLPKMAEKDGCFQSGFNEETCLVKIITGLLEFEVYLEYLQNRFESSEEQARAVQMSTKVLIQFLQKKAKNLDAITTPDPTTNASLLTKLQAQNQWLQDMTTHLILRSFKEFLQSSLRALRQM

SEQ ID NO: 2 - DNA, 1127 bp, Homo sapiens
  source 1..1127: mol_type = other DNA
attctgccctcgagcccaccgggaacgaaagagaagctctatctcccctccaggagcccagctatgaactccttctccacaagcgccttcggtccagttgccttctccctggggctgctcctggtgttgcctgctgccttccctgccccagtacccccaggagaagattccaaagatgtagccgccccacacagacagccactcacctcttcagaacgaattgacaaacaaattcggtacatcctcgacggcatctcagccctgagaaaggagacatgtaacaagagtaacatgtgtgaaagcagcaaagaggcactggcagaaaacaacctgaaccttccaaagatggctgaaaaagatggatgcttccaatctggattcaatgaggagacttgcctggtgaaaatcatcactggtcttttggagtttgaggtatacctagagtacctccagaacagatttgagagtagtgaggaacaagccagagctgtgcagatgagtacaaaagtcctgatccagttcctgcagaaaaaggcaaagaatctagatgcaataaccacccctgacccaaccacaaatgccagcctgctgacgaagctgcaggcacagaaccagtggctgcaggacatgacaactcatctcattctgcgcagctttaaggagttcctgcagtccagcctgagggctcttcggcaaatgtagcatgggcacctcagattgttgttgttaatgggcattccttcttctggtcagaaacctgtccactgggcacagaacttatgttgttctctatggagaactaaaagtatgagcgttaggacactattttaattatttttaatttattaatatttaaatatgtgaagctgagttaatttatgtaagtcatatttatatttttaagaagtaccacttgaaacattttatgtattagttttgaaataataatggaaagtggctatgcagtttgaatatcctttgtttcagagccagatcatttcttggaaagtgtaggcttacctcaaataaatggctaacttatacatatttttaaagaaatatttatattgtatttatataatgtataaatggtttttataccaataaatggcattttaaaaaattca

SEQ ID NO: 3 - protein, 610 aa, Homo sapiens
MIASQFLSALTLVLLIKESGAWSYNTSTEAMTYDEASAYCQQRYTHLVAIQNKEEIEYLNSILSYSPSYYWIGIRKVNNVWVWVGTQKPLTEEAKNWAPGEPNNRQKDEDCVEIYIKREKDVGMWNDERCSKKKLALCYTAACTNTSCSGHGECVETINNYTCKCDPGFSGLKCEQIVNCTALESPEHGSLVCSHPLGNFSYNSSCSISCDRGYLPSSMETMQCMSSGEWSAPIPACNVVECDAVTNPANGFVECFQNPGSFPWNTTCTFDCEEGFELMGAQSLQCTSSGNWDNEKPTCKAVTCRAVRQPQNGSVRCSHSPAGEFTFKSSCNFTCEEGFMLQGPAQVECTTQGQWTQQIPVCEAFQCTALSNPERGYMNCLPSASGSFRYGSSCEFSCEQGFVLKGSKRLQCGPTGEWDNEKPTCEAVRCDAVHQPPKGLVRCAHSPIGEFTYKSSCAFSCEEGFELHGSTQLECTSQGQWTEEVPSCQVVKCSSLAVPGKINMSCSGEPVFGTVCKFACPEGWTLNGSAARTCGATGHWSGLLPTCEAPTESNIPLVAGLSAAGLSLLTLAPFLLWLRKCLRKAKKFVPASSCQSLESDGSYQKPSYIL

SEQ ID NO: 4 - DNA, 3875 bp, Homo sapiens
  source 1..3875: mol_type = other DNA
agctgttcttggctgacttcacatcaaaactcctatactgacctgagacagaggcagcagtgatacccacctgagagatcctgtgtttgaacaactgcttcccaaaacggaaagtatttcaagcctaaacctttgggtgaaaagaactcttgaagtcatgattgcttcacagtttctctcagctctcactttggtgcttctcattaaagagagtggagcctggtcttacaacacctccacggaagctatgacttatgatgaggccagtgcttattgtcagcaaaggtacacacacctggttgcaattcaaaacaaagaagagattgagtacctaaactccatattgagctattcaccaagttattactggattggaatcagaaaagtcaacaatgtgtgggtctgggtaggaacccagaaacctctgacagaagaagccaagaactgggctccaggtgaacccaacaataggcaaaaagatgaggactgcgtggagatctacatcaagagagaaaaagatgtgggcatgtggaatgatgagaggtgcagcaagaagaagcttgccctatgctacacagctgcctgtaccaatacatcctgcagtggccacggtgaatgtgtagagaccatcaataattacacttgcaagtgtgaccctggcttcagtggactcaagtgtgagcaaattgtgaactgtacagccctggaatcccctgagcatggaagcctggtttgcagtcacccactgggaaacttcagctacaattcttcctgctctatcagctgtgataggggttacctgccaagcagcatggagaccatgcagtgtatgtcctctggagaatggagtgctcctattccagcctgcaatgtggttgagtgtgatgctgtgacaaatccagccaatgggttcgtggaatgtttccaaaaccctggaagcttcccatggaacacaacctgtacatttgactgtgaagaaggatttgaactaatgggagcccagagccttcagtgtacctcatctgggaattgggacaacgagaagccaacgtgtaaagctgtgacatgcagggccgtccgccagcctcagaatggctctgtgaggtgcagccattcccctgctggagagttcaccttcaaatcatcctgcaacttcacctgtgaggaaggcttcatgttgcagggaccagcccaggttgaatgcaccactcaagggcagtggacacagcaaatcccagtttgtgaagctttccagtgcacagccttgtccaaccccgagcgaggctacatgaattgtcttcctagtgcttctggcagtttccgttatgggtccagctgtgagttctcctgtgagcagggttttgtgttgaagggatccaaaaggctccaatgtggccccacaggggagtgggacaacgagaagcccacatgtgaagctgtgagatgcgatgctgtccaccagcccccgaagggtttggtgaggtgtgctcattcccctattggagaattcacctacaagtcctcttgtgccttcagctgtgaggagggatttgaattacatggatcaactcaacttgagtgcacatctcagggacaatggacagaagaggttccttcctgccaagtggtaaaatgttcaagcctggcagttccgggaaagatcaacatgagctgcagtggggagcccgtgtttggcactgtgtgcaagttcgcctgtcctgaaggatggacgctcaatggctctgcagctcggacatgtggagccacaggacactggtctggcctgctacctacctgtgaagctcccactgagtccaacattcccttggtagctggactttctgctgctggactctccctcctgacattagcaccatttctcctctggcttcggaaatgcttacggaaagcaaagaaatttgttcctgccagcagctgccaaagccttgaatcagatggaagctaccaaaagccttcttacatcctttaagttcaaaagaatcagaaacaggtgcatctggggaactagagggatacactgaagttaacagagacagataactctcctcgggtctctggcccttcttgcctactatgccagatgcctttatggctgaaaccgcaacacccatcaccacttcaatagatcaaagtccagcaggcaaggacggccttcaactgaaaagactcagtgttccctttcctactctcaggatcaagaaagtgttggctaatgaagggaaaggatattttcttccaagcaaaggtgaagagaccaagactctgaaatctcagaattccttttctaactctcccttgctcgctgtaaaatcttggcacagaaacacaatattttgtggctttctttcttttgcccttcacagtgtttcgacagctgattacacagttgctgtcataagaatgaataataattatccagagtttagaggaaaaaaatgactaaaaatattataacttaaaaaaatgacagatgttgaatgcccacaggcaaatgcatggagggttgttaatggtgcaaatcctactgaatgctctgtgcgagggttactatgcacaatttaatcactttcatccctatgggattcagtgcttcttaaagagttcttaaggattgtgatatttttacttgcattgaatatattataatcttccatacttcttcattcaatacaagtgtggtagggacttaaaaaacttgtaaatgctgtcaactatgatatggtaaaagttacttattctagattaccccctcattgtttattaacaaattatgttacatctgttttaaatttatttcaaaaagggaaactattgtcccctagcaaggcatgatgttaaccagaataaagttctgagtgtttttactacagttgttttttgaaaacatggtagaattggagagtaaaaactgaatggaaggtttgtatattgtcagatattttttcagaaatatgtggtttccacgatgaaaaacttccatgaggccaaacgttttgaactaataaaagcataaatgcaaacacacaaaggtataattttatgaatgtctttgttggaaaagaatacagaaagatggatgtgctttgcattcctacaaagatgtttgtcagatatgatatgtaaacataattcttgtatattatggaagattttaaattcacaatagaaactcaccatgtaaaagagtcatctggtagatttttaacgaatgaagatgtctaatagttattccctatttgttttcttctgtatgttagggtgctctggaagagaggaatgcctgtgtgagcaagcatttatgtttatttataagcagatttaacaattccaaaggaatctccagttttcagttgatcactggcaatgaaaaattctcagtcagtaattgccaaagctgctctagccttgaggagtgtgagaatcaaaactctcctacacttccattaacttagcatgtgttgaaaaaaaagtttcagagaagttctggctgaacactggcaacaacaaagccaacagtcaaaacagagatgtgataaggatcagaacagcagaggttcttttaaaggggcagaaaaactctgggaaataagagagaacaactactgtgatcaggctatgtatggaatacagtgttattttctttgaaattgtttaagtgttgtaaatatttatgtaaactgcattagaaattagctgtgtgaaataccagtgtggtttgtgtttgagttttattgagaattttaaattataacttaaaatattttataatttttaaagtatatatttatttaagcttatgtcagacctatttgacataacactataaaggttgacaataaatgtgcttatgttta

SEQ ID NO: 5 - protein, 338 aa, Homo sapiens
MFQAFPGDYDSGSRCSSSPSAESQYLSSVDSFGSPPTAAASQECAGLGEMPGSFVPTVTAITTSQDLQWLVQPTLISSMAQSQGQPLASQPPVVDPYDMPGTSYSTPGMSGYSSGGASGSGGPSTSGTTSGPGPARPARARPRRPREETLTPEEEEKRRVRRERNKLAAAKCRNRRRELTDRLQAETDQLEEEKAELESEIAELQKEKERLEFVLVAHKPGCKIPYEEGPGPGPLAEVRDLPGSAPAKEDGFSWLLPPPPPPPLPFQTSQDAPPNLTASLFTHSEVQVLGDPFPVVNPSYTSSFVLTCPEVSAFAGAQRTSGSDQPSDPLNSPSLLAL

SEQ ID NO: 6 - DNA, 3775 bp, Homo sapiens
  source 1..3775: mol_type = other DNA
attcataagactcagagctacggccacggcagggacacgcggaaccaagacttggaaacttgattgttgtggttcttcttgggggttatgaaatttcattaatctttttttttccggggagaaagtttttggaaagattcttccagatatttcttcattttcttttggaggaccgacttactttttttggtcttctttattactcccctccccccgtgggacccgccggacgcgtggaggagaccgtagctgaagctgattctgtacagcgggacagcgctttctgcccctgggggagcaacccctccctcgcccctgggtcctacggagcctgcactttcaagaggtacagcggcatcctgtgggggcctgggcaccgcaggaagactgcacagaaactttgccattgttggaacgggacgttgctccttccccgagcttccccggacagcgtactttgaggactcgctcagctcaccggggactcccacggctcaccccggacttgcaccttacttccccaacccggccatagccttggcttcccggcgacctcagcgtggtcacaggggcccccctgtgcccagggaaatgtttcaggctttccccggagactacgactccggctcccggtgcagctcctcaccctctgccgagtctcaatatctgtcttcggtggactccttcggcagtccacccaccgccgccgcctcccaggagtgcgccggtctcggggaaatgcccggttccttcgtgcccacggtcaccgcgatcacaaccagccaggacctccagtggcttgtgcaacccaccctcatctcttccatggcccagtcccaggggcagccactggcctcccagcccccggtcgtcgacccctacgacatgccgggaaccagctactccacaccaggcatgagtggctacagcagtggcggagcgagtggcagtggtgggccttccaccagcggaactaccagtgggcctgggcctgcccgcccagcccgagcccggcctaggagaccccgagaggagacgctcaccccagaggaagaggagaagcgaagggtgcgccgggaacgaaataaactagcagcagctaaatgcaggaaccggcggagggagctgaccgaccgactccaggcggagacagatcagttggaggaagaaaaagcagagctggagtcggagatcgccgagctccaaaaggagaaggaacgtctggagtttgtgctggtggcccacaaaccgggctgcaagatcccctacgaagaggggcccgggccgggcccgctggcggaggtgagagatttgccgggctcagcaccggctaaggaagatggcttcagctggctgctgccgcccccgccaccaccgcccctgcccttccagaccagccaagacgcaccccccaacctgacggcttctctctttacacacagtgaagttcaagtcctcggcgaccccttccccgttgttaacccttcgtacacttcttcgtttgtcctcacctgcccggaggtctccgcgttcgccggcgcccaacgcaccagcggcagtgaccagccttccgatcccctgaactcgccctccctcctcgctctgtgaactctttagacacacaaaacaaacaaacacatgggggagagagacttggaagaggaggaggaggaggagaaggaggagagagaggggaagagacaaagtgggtgtgtggcctccctggctcctccgtctgaccctctgcggccactgcgccactgccatcggacaggaggattccttgtgttttgtcctgcctcttgtttctgtgccccggcgaggccggagagctggtgactttggggacagggggtgggaaggggatggacacccccagctgactgttggctctctgacgtcaacccaagctctggggatgggtggggaggggggcgggtgacgcccaccttcgggcagtcctgtgtgaggattaagggacgggggtgggaggtaggctgtggggtgggctggagtcctctccagagaggctcaacaaggaaaaatgccactccctacccaatgtctcccacacccaccctttttttggggtgcctaggttggtttcccctgcactcccgaccttagcttattgatcccacatttccatggtgtgagatcctctttactctgggcagaagtgagccccccccttaaagggaattcgatgcccccctagaataatctcatccccccacccgacttcttttgaaatgtgaacgtccttccttgactgtctagccactccctcccagaaaaactggctctgattggaatttctggcctcctaaggctccccaccccgaaatcagcccccagccttgtttctgatgacagtgttatcccaagaccctgccccctgccagccgaccctcctggccttcctcgttgggccgctctgatttcaggcagcaggggctgctgtgatgccgtcctgctggagtgatttatactgtgaaatgagttggccagattgtggggtgcagctgggtggggcagcacacctctggggggataatgtccccactcccgaaagcctttcctcggtctcccttccgtccatcccccttcttcctcccctcaacagtgagttagactcaagggggtgacagaaccgagaagggggtgacagtcctccatccacgtggcctctctctctctcctcaggaccctcagccctggcctttttctttaaggtcccccgaccaatccccagcctaggacgccaacttctcccaccccttggcccctcacatcctctccaggaagggagtgaggggctgtgacatttttccggagaagatttcagagctgaggctttggtacccccaaacccccaatatttttggactggcagactcaaggggctggaatctcatgattccatgcccgagtccgcccatccctgaccatggttttggctctcccaccccgccgttccctgcgcttcatctcatgaggatttctttatgaggcaaatttatattttttaatatcggggggtggaccacgccgccctccatccgtgctgcatgaaaaacattccacgtgccccttgtcgcgcgtctcccatcctgatcccagacccattccttagctatttatccctttcctggtttccgaaaggcaattatatctattatgtataagtaaatatattatatatggatgtgtgtgtgtgcgtgcgcgtgagtgtgtgagcgcttctgcagcctcggcctaggtcacgttggccctcaaagcgagccgttgaattggaaactgcttctagaaactctggctcagcctgtctcgggctgacccttttctgatcgtctcggcccctctgattgttcccgatggtctctctccctctgtcttttctcctccgcctgtgtccatctgaccgttttcacttgtctcctttctgactgtccctgccaatgctccagctgtcgtctgactctgggttcgttggggacatgagattttattttttgtgagtgagactgagggatcgtagatttttacaatctgtatctttgacaattctgggtgcgagtgtgagagtgtgagcagggcttgctcctgccaaccacaattcaatgaatccccgacccccctaccccatgctgtacttgtggttctctttttgtattttgcatctgaccccggggggctgggacagattggcaatgggccgtcccctctccccttggttctgcactgttgccaataaaaagctcttaaaaacgca

SEQ ID NO: 7 - protein, 1582 aa, Homo sapiens
MAGPGGWRDREVTDLGHLPDPTGIFSLDKTIGLGTYGRIYLGLHEKTGAFTAVKVMNARKTPLPEIGRRVRVNKYQKSVGWRYSDEEEDLRTELNLLRKYSFHKNIVSFYGAFFKLSPPGQRHQLWMVMELCAAGSVTDVVRMTSNQSLKEDWIAYICREILQGLAHLHAHRVIHRDIKGQNVLLTHNAEVKLVDFGVSAQVSRTNGRRNSFIGTPYWMAPEVIDCDEDPRRSYDYRSDVWSVGITAIEMAEGAPPLCNLQPLEALFVILRESAPTVKSSGWSRKFHNFMEKCTIKNFLFRPTSANMLQHPFVRDIKNERHVVESLTRHLTGIIKKRQKKGIPLIFEREEAIKEQYTVRRFRGPSCTHELLRLPTSSRCRPLRVLHGEPSQPRWLPDREEPQVQALQQLQGAARVFMPLQALDSAPKPLKGQAQAPQRLQGAARVFMPLQAQVKAKASKPLQMQIKAPPRLRRAARVLMPLQAQVRAPRLLQVQSQVSKKQQAQTQTSEPQDLDQVPEEFQGQDQVPEQQRQGQAPEQQQRHNQVPEQELEQNQAPEQPEVQEQAAEPAQAETEAEEPESLRVNAQVFLPLLSQDHHVLLPLHLDTQVLIPVEGQTEGSPQAQAWTLEPPQAIGSVQALIEGLSRDLLRAPNSNNSKPLGPLQTLMENLSSNRFYSQPEQAREKKSKVSTLRQALAKRLSPKRFRAKSSWRPEKLELSDLEARRQRRQRRWEDIFNQHEEELRQVDKDKEDESSDNDEVFHSIQAEVQIEPLKPYISNPKKIEVQERSPSVPNNQDHAHHVKFSSSVPQRSLLEQAQKPIDIRQRSSQNRQNWLAASESSSEEESPVTGRRSQSSPPYSTIDQKLLVDIHVPDGFKVGKISPPVYLTNEWVGYNALSEIFRNDWLTPAPVIQPPEEDGDYVELYDASADTDGDDDDESNDTFEDTYDHANGNDDLDNQVDQANDVCKDHDDDNNKFVDDVNNNYYEAPSCPRASYGRDGSCKQDGYDGSRGKEEAYRGYGSHTANRSHGGSAASEDNAAIGDQEEHAANIGSERRGSEGDGGKGVVRTSEESGALGLNGEENCSETDGPGLKRPASQDFEYLQEEPGGGNEASNAIDSGAAPSAPDHESDNKDISESSTQSDFSANHSSPSKGSGMSADANFASAILYAGFVEVPEESPKQPSEVNVNPLYVSPACKKPLIHMYEKEFTSEICCGSLWGVNLLLGTRSNLYLMDRSGKADITKLIRRRPFRQIQVLEPLNLLITISGHKNRLRVYHLTWLRNKILNNDPESKRRQEEMLKTEEACKAIDKLTGCEHFSVLQHEETTYIAIALKSSIHLYAWAPKSFDESTAIKVCIDQSADSEGDYMSYQAYIRILAKIQAADPVNRFKRPDELLHLLKLKVFPTLDHKPVTVDLAIGSEKRLKIFFSSADGYHLIDAESEVMSDVTLPKNPLEIIIPQNIIILPDCLGIGMMLTFNAEALSVEANEQLFKKILEMWKDIPSSIAFECTQRTTGWGQKAIEVRSLQSRVLESELKRRSIKKLRFLCTRGDKLFFTSTLRNHHSRVYFMTLGKLEELQSNYDV

SEQ ID NO: 8 - DNA, 8066 bp, Homo sapiens
  source 1..8066: mol_type = other DNA
agacacagcgctctcgacacggagcacccttctagcttcttcgtctccaggactgacgctcaggctcctctctcgccttagcccaacttgctttcccgcctcgcaaactccggtttccctccactcccaactcttttcactacacgtttcccctcctctatctcccacgccacgaaccccgatccccagactcctctctcccgccctcctccttcctctctcctcccttcaactcttcatccgcttccacctcagactctgcgcgcacccaattcagtcgcccgctcccgttcggctcctcgaagccatggcgggacctgggggctggagggacagggaggtcacggatctgggccacctgccggatccaactggaatattctcactagataaaaccattggccttggtacttatggcagaatctatttgggacttcatgagaagactggtgcatttacagctgttaaagtgatgaacgctcgtaagacccctttacctgaaataggaaggcgagtgagagtgaataaatatcaaaaatctgttgggtggagatacagtgatgaggaagaggatctcaggactgaactcaaccttctgaggaagtactctttccacaaaaacattgtgtccttctatggagcatttttcaagctgagtccccctggtcagcggcaccaactttggatggtgatggagttatgtgcagcaggttcggtcactgatgtagtgagaatgaccagtaatcagagtttaaaagaagattggattgcttatatctgccgagaaatccttcagggcttagctcaccttcacgcacaccgagtaattcaccgggacatcaaaggtcagaatgtgctgctgactcataatgctgaagtaaaactggttgattttggagtgagtgcccaggtgagcagaactaatggaagaaggaatagtttcattgggacaccatactggatggcacctgaggtgattgactgtgatgaggacccaagacgctcctatgattacagaagtgatgtgtggtctgtgggaattactgccattgaaatggctgaaggagcccctcctctgtgtaaccttcaacccttggaagctctcttcgttattttgcgggaatctgctcccacagtcaaatccagcggatggtcccgtaagttccacaatttcatggaaaagtgtacgataaaaaatttcctgtttcgtcctacttctgcaaacatgcttcaacacccatttgttcgggatataaaaaatgaacgacatgttgttgagtcattaacaaggcatcttactggaatcattaaaaaaagacagaaaaaaggaatacctttgatctttgaaagagaagaagctattaaggaacagtacaccgtgagaagattcagaggaccctcttgcactcacgagcttctgagattgccaaccagcagcagatgcagaccacttagagtcctgcatggggaaccctctcagccaaggtggctacctgatcgagaagagccacaggtccaggcacttcagcagctacagggagcagccagggtattcatgccactgcaggctctggacagtgcacctaagcctctaaaggggcaggctcaggcacctcaacgactacaaggggcagctcgggtgttcatgccactacaggctcaggtgaaggctaaggcctctaaacctctacaaatgcagattaaggcacctccacgactacggagggcagccagggtgctcatgccactacaggcacaggttagggcacctaggcttctgcaggtacagtcccaggtatccaaaaagcagcaggcccagacccagacatcagaaccacaagatttggaccaggtaccagaggaatttcagggtcaagatcaggtacccgaacaacaaaggcagggccaggcccctgaacaacagcagaggcacaaccaggtgcctgaacaagagctggagcagaaccaggcacctgaacagccagaggtacaggaacaggctgccgagcctgcacaggcagagactgaggcagaggaacctgagtcattacgagtaaatgcccaggtatttctgcccctgctatcacaagatcaccatgtgctgttgccactacatttggatactcaggtgctcattccagtagaggggcaaactgaaggatcacctcaggcacaggcttggacactagaacccccacaggcaattggctcagttcaagcactgatagagggactatcaagagacttgcttcgggcaccaaactcaaataactcaaagccacttggtccgttgcaaaccctgatggaaaatctgtcatcaaataggttttactcacaaccagaacaggcacgggagaaaaaatcaaaagtttctactctgaggcaagcactggcaaaaagactatcaccaaagaggttcagggcaaagtcatcatggagacctgaaaagcttgaactctcggatttagaagcccgcaggcaaaggcgccaacgcagatgggaagatatctttaatcagcatgaggaagaattgagacaagttgataaagacaaagaagatgaatcatcagacaatgatgaagtatttcattcgattcaggctgaagtccagatagagccattgaagccatacatttcaaatcctaaaaaaattgaggttcaagagagatctccttctgtgcctaacaaccaggatcatgcacatcatgtcaagttctcttcaagcgttcctcagcggtctcttttggaacaagctcagaagcccattgacatcagacaaaggagttcgcaaaatcgtcaaaattggctggcagcatcagaatcttcttctgaggaagaaagtcctgtgactggaaggaggtctcagtcatcaccaccttattctactattgatcagaagttgctggttgacatccatgttccagatggatttaaagtaggaaaaatatcaccccctgtatacttgacaaacgaatgggtaggctataatgcactctctgaaatcttccggaatgattggttaactccggcacctgtcattcagccacctgaagaggatggtgattatgttgaactctatgatgccagtgctgatactgatggtgatgatgatgatgagtctaatgatacttttgaagatacctatgatcatgccaatggcaatgatgacttggataaccaggttgatcaggctaatgatgtttgtaaagaccatgatgatgacaacaataagtttgttgatgatgtaaataataattattatgaggcgcctagttgtccaagggcaagctatggcagagatggaagctgcaagcaagatggttatgatggaagtcgtggaaaagaggaagcctacagaggctatggaagccatacagccaatagaagccatggaggaagtgcagccagtgaggacaatgcagccattggagatcaggaagaacatgcagccaatataggcagtgaaagaagaggcagtgagggtgatggaggtaagggagtcgttcgaaccagtgaagagagtggagcccttggactcaatggagaagaaaattgctcagagacagatggtccaggattgaagagacctgcgtctcaggactttgaatatctacaggaggagccaggtggtggaaatgaggcctcaaatgccattgactcaggtgctgcaccgtcagcacctgatcatgagagtgacaataaggacatatcagaatcatcaacacaatcagatttttctgccaatcactcatctccttccaaaggttctgggatgtctgctgatgctaactttgccagtgccatcttatacgctggattcgtagaagtacctgaggaatcacctaagcaaccctctgaagtcaatgttaacccactctatgtctctcctgcatgtaaaaaaccactaatccacatgtatgaaaaggagttcacttctgagatctgctgtggttctttgtggggagtcaatttgctgttgggaacccgatctaatctatatctgatggacagaagtggaaaggctgacattactaaacttataaggcgaagaccattccgccagattcaagtcttagagccactcaatttgctgattaccatctcaggtcataagaacagacttcgggtgtatcatctgacctggttgaggaacaagattttgaataatgatccagaaagtaaaagaaggcaagaagaaatgctgaagacagaggaagcctgcaaagctattgataagttaacaggctgtgaacacttcagtgtcctccaacatgaagaaacaacatatattgcaattgctttgaaatcatcaattcacctttatgcatgggcaccaaagtcctttgatgaaagcactgctattaaagtatgcattgatcaatcagcagactctgaaggagactacatgtcctatcaagcctatatacgaatactggcaaaaatacaggcagctgatccagtgaaccggtttaagagaccagatgagctccttcatttgctgaagctcaaggtatttccaacacttgatcataagccagtgacagttgacctggctattggttctgaaaaaagactaaagattttcttcagctcagcagatggatatcacctcatcgatgcagaatctgaggttatgtctgatgtgaccctgccaaagaatcccctggaaatcattataccacagaatatcatcattttacctgattgcttgggaattggcatgatgctcaccttcaatgctgaagccctctctgtggaagcaaatgaacaactcttcaagaagatccttgaaatgtggaaagacataccatcttctatagcttttgaatgtacacagcgaaccacaggatggggccaaaaggccattgaagtgcgctctttgcaatccagggttctggaaagtgagctgaagcgcaggtcaattaagaagctgagattcctgtgcacccggggtgacaagctgttctttacctctaccctgcgcaatcaccacagccgggtttacttcatgacacttggaaaacttgaagagctccaaagcaattatgatgtctaaaagtttccagtgatttattaccacattataaacatcatgtataggcagtctgcatcttcagatttcagagattaaatgagtattcagttttatttttagtaaagattaaatccaaaactttacttttaatgtagcacagaatagttttaatgagaaatgcagctttatgtataaaattaactatagcaagctctaggtactccaatggtgtacaatgtcttttgcacaaactttgtaacttttgttactgtgaattcaaacattactctttggacagtttggacagtatctgtattcagattttacaacatggagtaaagaaacctgttatgaattagattacaagcagccttcaaaagaattggcactgggataagatttttcagaaaaagaaaaacatcggcaaactgtgtgtgatttttccaaagctatataaagaaccaaaggtttagtcaagaaacaaaaatcttaaagattattataacccagactaaggttgaacaacctgcatgcccagagaaaactatggcgacaaaggggaaaaggccaccactcgttttctcactgattcatgccaattaagcctacagttaaagaccagttttgttcttttcacccatttttaagctggttttctcctgataagaagaaaggaagaaagccccagacgcttggtttttctcagaacccccaaaagatgtgcaatagctgttgttacaaaccaccaaataatacagttgtgagcctgaatacaggactgaactcctatacacgtgtactgtagaatgagtattttttaataccttaaggtaggcgtcaaattctactccccaaagcagagatagattgatttatcaaaattattatctggccaacagtgtgactatcagacagcatcaaatatttgcccaatccaagattagactacacaaaagcttccttccagtattaaacaaaaagaattaaacataactatgaaaaaactttgctaatatctgtgtttttcagatttcattttttgtaaaatcagaaattaatctaaacatattcagtgataagttcatgtgtaacgacttaatgttaaaggttaaaaaaaagatttcacaaaatatacaactttcaccatatatataagcctgcaaaattagagtagtgaaagtcatgctagtccatcacccaaatatgttatagacgccatagacaggtgatgtttggtcacctatggtaactgctacctgatgaagagcataatttctgcatatccatcctcaataccatggtaaattctggggcaatagagaagcaacagaactgccacaaagtatacctcaatataattcctctagttctgcttctaaaatctgaggacagtgctagtgggaaaataattttcaaactacctggttaaccaaaatacaaaagcagctgactatgtgtgatttcataatagcacatttcttgacacttagtgctagaaatgaagatttggattttcctaacaacttacatcaagaatgtagtgtagctcattattgagaatttaggaaagcctgaatccattaattaaggaaataaatgtgactcacatttcttttactgtgacacaataatgtgatcctaaaactggcttatccttgagtgtttacaactcaaacaactttttgaatgcagtagttttttttttttaaaaacaaacttttatgtcaaattttttttcttagaagtagtcttcattattataaatttgtacaccaaaaggccatggggaactttgtgcaagtacctcatcgctgagcaaatggagcttgctatgttttaatttcagaaaatttcctcatatacgtagtgtgtagaatcaagtcttttaataattcattttttcttcataatatttactcaaagttaagcttaaaaataagttttatcttaaaatcatatttgaagacagtaagacagtaaactattttaggaagtcaacccccattgcactctgtggcagttattctggtaaaaataggcaaaagtgacctgaatctacaatgatgtcccaaagtaaccaagtaagagagattgtaaatgataaaccgagctttaaaggataaagtgttaataaagaaaggaagctgggcacatgtcaaaaagggagatcgaaatgttaggtaatcatttagaaaggacagaaaatatttaaagtggctcataggtaatgaatatttctgacttagatgtaaatccatctggaatctttacatcctttgccagctgaaacaagaaagtgaagggacaatgatatttcatggtcagtttattttgtaagagacagaagaaattatatctatacattaccttgtagcagcagtacctggaagccccagcccgtcacagaagtgtggaggggggctcctgactagacaatttccctagcccttgtgatttgaagcatgaaagttctggcaggttatgagcagcactagggataaagtatggttttattttggtgtaatttaggtttttcaacaaagcccttgtctaaaataaaaggcattattggaaatatttgaaaactagaaaatgatggataaaagggctgataagaaaatttctgactgtcagtagaagtgagataagatcctcagaggaaacagtaagaagggataatcattaagatagtaaaacaggcaaagcagaatcacatgtgcacacacacatacacatgtaaacattggaatgcataagttttaatattttagcgctatcagtttctaaatgcattaattactaactgccctctcccaagattcatttagttcaaacagtatccgtaaactaggaataatgccacatgcattcaatgggatcttttaagtactcttcagtttgttccaagaaatgtgcctactgaaatcaaattaatttgtattcaatgtgtacttcaagactgctaattgtttcatctgaaagcctacaatgaatcattgttcaaccttgaaaaataaaattttgtaaaaca

SEQ ID NO: 9 - protein, 900 aa, Homo sapiens
MESCYNPGLDGIIEYDDFKLNSSIVEPKEPAPETADGPYLVIVEQPKQRGFRFRYGCEGPSHGGLPGASSEKGRKTYPTVKICNYEGPAKIEVDLVTHSDPPRAHAHSLVGKQCSELGICAVSVGPKDMTAQFNNLGVLHVTKKNMMGTMIQKLQRQRLRSRPQGLTEAEQRELEQEAKELKKVMDLSIVRLRFSAFLRASDGSFSLPLKPVISQPIHDSKSPGASNLKISRMDKTAGSVRGGDEVYLLCDKVQKDDIEVRFYEDDENGWQAFGDFSPTDVHKQYAIVFRTPPYHKMKIERPVTVFLQLKRKRGGDVSDSKQFTYYPLVEDKEEVQRKRRKALPTFSQPFGGGSHMGGGSGGAAGGYGGAGGGGSLGFFPSSLAYSPYQSGAGPMGCYPGGGGGAQMAATVPSRDSGEEAAEPSAPSRTPQCEPQAPEMLQRAREYNARLFGLAQRSARALLDYGVTADARALLAGQRHLLTAQDENGDTPLHLAIIHGQTSVIEQIVYVIHHAQDLGVVNLTNHLHQTPLHLAVITGQTSVVSFLLRVGADPALLDRHGDSAMHLALRAGAGAPELLRALLQSGAPAVPQLLHMPDFEGLYPVHLAVRARSPECLDLLVDSGAEVEATERQGGRTALHLATEMEELGLVTHLVTKLRANVNARTFAGNTPLHLAAGLGYPTLTRLLLKAGADIHAENEEPLCPLPSPPTSDSDSDSEGPEKDTRSSFRGHTPLDLTCSTKVKTLLLNAAQNTMEPPLTPPSPAGPGLSLGDTALQNLEQLLDGPEAQGSWAELAERLGLRSLVDTYRQTTSPSGSLLRSYELAGGDLAGLLEALSDMGLEEGVRLLRGPETRDKLPSTAEVKEDSAYGSQSVEQEAEKLGPPPEPPGGLCHGHPQPQVH

SEQ ID NO: 10 - DNA, 3125 bp, Homo sapiens
  source 1..3125: mol_type = other DNA
ccgcaaccagagccgccgccacggtgagtggctggattcagacccctgggtggccgggacaagagaaaagagggaggagggcctttagcggacagcgcctggggctggagagcagcagctgcacacagccggaaagggcgcgcaggcgacgacactcggatccacgtcgacaccgttgtacaaagatacgcggacccgcgggcgtctaaaattctgggaagcagaacctggccggagccactagacagagccgggcctagcccagagacatggagagttgctacaacccaggtctggatggtattattgaatatgatgatttcaaattgaactcctccattgtggaacccaaggagccagccccagaaacagctgatggcccctacctggtgatcgtggaacagcctaagcagagaggcttccgatttcgatatggctgtgaaggcccctcccatggaggactgcccggtgcctccagtgagaagggccgaaagacctatcccactgtcaagatctgtaactacgagggaccagccaagatcgaggtggacctggtaacacacagtgacccacctcgtgctcatgcccacagtctggtgggcaagcaatgctcggagctggggatctgcgccgtttctgtggggcccaaggacatgactgcccaatttaacaacctgggtgtcctgcatgtgactaagaagaacatgatggggactatgatacaaaaacttcagaggcagcggctccgctctaggccccagggccttacggaggccgagcagcgggagctggagcaagaggccaaagaactgaagaaggtgatggatctgagtatagtgcggctgcgcttctctgccttccttagagccagtgatggctccttctccctgcccctgaagccagtcatctcccagcccatccatgacagcaaatctccgggggcatcaaacctgaagatttctcgaatggacaagacagcaggctctgtgcggggtggagatgaagtttatctgctttgtgacaaggtgcagaaagatgacattgaggttcggttctatgaggatgatgagaatggatggcaggcctttggggacttctctcccacagatgtgcataaacagtatgccattgtgttccggacacccccctatcacaagatgaagattgagcggcctgtaacagtgtttctgcaactgaaacgcaagcgaggaggggacgtgtctgattccaaacagttcacctattaccctctggtggaagacaaggaagaggtgcagcggaagcggaggaaggccttgcccaccttctcccagcccttcgggggtggctcccacatgggtggaggctctgggggtgcagccgggggctacggaggagctggaggaggtggcagcctcggtttcttcccctcctccctggcctacagcccctaccagtccggcgcgggccccatgggctgctacccgggaggcgggggcggggcgcagatggccgccacggtgcccagcagggactccggggaggaagccgcggagccgagcgccccctccaggaccccccagtgcgagccgcaggccccggagatgctgcagcgagctcgagagtacaacgcgcgcctgttcggcctggcgcagcgcagcgcccgagccctactcgactacggcgtcaccgcggacgcgcgcgcgctgctggcgggacagcgccacctgctgacggcgcaggacgagaacggagacacaccactgcacctagccatcatccacgggcagaccagtgtcattgagcagatagtctatgtcatccaccacgcccaggacctcggcgttgtcaacctcaccaaccacctgcaccagacgcccctgcacctggcggtgatcacggggcagacgagtgtggtgagctttctgctgcgggtaggtgcagacccagctctgctggatcggcatggagactcagccatgcatctggcgctgcgggcaggcgctggtgctcctgagctgctgcgtgcactgcttcagagtggagctcctgctgtgccccagctgttgcatatgcctgactttgagggactgtatccagtacacctggcggtccgagcccgaagccctgagtgcctggatctgctggtggacagtggggctgaagtggaggccacagagcggcaggggggacgaacagccttgcatctagccacagagatggaggagctggggttggtcacccatctggtcaccaagctccgggccaacgtgaacgctcgcacctttgcgggaaacacacccctgcacctggcagctggactggggtacccgaccctcacccgcctccttctgaaggctggtgctgacatccatgctgaaaacgaggagcccctgtgcccactgccttcaccccctacctctgatagcgactcggactctgaagggcctgagaaggacacccgaagcagcttccggggccacacgcctcttgacctcacttgcagcaccaaggtgaagaccttgctgctaaatgctgctcagaacaccatggagccacccctgaccccgcccagcccagcagggccgggactgtcacttggtgatacagctctgcagaacctggagcagctgctagacgggccagaagcccagggcagctgggcagagctggcagagcgtctggggctgcgcagcctggtagacacgtaccgacagacaacctcacccagtggcagcctcctgcgcagctacgagctggctggcggggacctggcaggtctactggaggccctgtctgacatgggcctagaggagggagtgaggctgctgaggggtccagaaacccgagacaagctgcccagcacagcagaggtgaaggaagacagtgcgtacgggagccagtcagtggagcaggaggcagagaagctgggcccaccccctgagccaccaggagggctctgccacgggcacccccagcctcaggtgcactgacctgctgcctgcccccagcccccttcccggaccccctgtacagcgtccccacctatttcaaatcttatttaacaccccacacccacccctcagttgggacaaataaaggattctcatgggaaggggaggacccctccttcccaacttatggca

SEQ ID NO: 11 - protein, 233 aa, Homo sapiens
MSTESMIRDVELAEEALPKKTGGPQGSRRCLFLSLFSFLIVAGATTLFCLLHFGVIGPQREEFPRDLSLISPLAQAVRSSSRTPSDKPVAHVVANPQAEGQLQWLNRRANALLANGVELRDNQLVVPSEGLYLIYSQVLFKGQGCPSTHVLLTHTISRIAVSYQTKVNLLSAIKSPCQRETPEGAEAKPWYEPIYLGGVFQLEKGDRLSAEINRPDYLDFAESGQVYFGIIAL

SEQ ID NO: 12 - DNA, 1678 bp, Homo sapiens
  source 1..1678: mol_type = other DNA
agcagacgctccctcagcaaggacagcagaggaccagctaagagggagagaagcaactacagaccccccctgaaaacaaccctcagacgccacatcccctgacaagctgccaggcaggttctcttcctctcacatactgacccacggctccaccctctctcccctggaaaggacaccatgagcactgaaagcatgatccgggacgtggagctggccgaggaggcgctccccaagaagacaggggggccccagggctccaggcggtgcttgttcctcagcctcttctccttcctgatcgtggcaggcgccaccacgctcttctgcctgctgcactttggagtgatcggcccccagagggaagagttccccagggacctctctctaatcagccctctggcccaggcagtcagatcatcttctcgaaccccgagtgacaagcctgtagcccatgttgtagcaaaccctcaagctgaggggcagctccagtggctgaaccgccgggccaatgccctcctggccaatggcgtggagctgagagataaccagctggtggtgccatcagagggcctgtacctcatctactcccaggtcctcttcaagggccaaggctgcccctccacccatgtgctcctcacccacaccatcagccgcatcgccgtctcctaccagaccaaggtcaacctcctctctgccatcaagagcccctgccagagggagaccccagagggggctgaggccaagccctggtatgagcccatctatctgggaggggtcttccagctggagaagggtgaccgactcagcgctgagatcaatcggcccgactatctcgactttgccgagtctgggcaggtctactttgggatcattgccctgtgaggaggacgaacatccaaccttcccaaacgcctcccctgccccaatccctttattaccccctccttcagacaccctcaacctcttctggctcaaaaagagaattgggggcttagggtcggaacccaagcttagaactttaagcaacaagaccaccacttcgaaacctgggattcaggaatgtgtggcctgcacagtgaagtgctggcaaccactaagaattcaaactggggcctccagaactcactggggcctacagctttgatccctgacatctggaatctggagaccagggagcctttggttctggccagaatgctgcaggacttgagaagacctcacctagaaattgacacaagtggaccttaggccttcctctctccagatgtttccagacttccttgagacacggagcccagccctccccatggagccagctccctctatttatgtttgcacttgtgattatttattatttatttattatttatttatttacagatgaatgtatttatttgggagaccggggtatcctgggggacccaatgtaggagctgccttggctcagacatgttttccgtgaaaacggagctgaacaataggctgttcccatgtagccccctggcctctgtgccttcttttgattatgttttttaaaatatttatctgattaagttgtctaaacaatgctgatttggtgaccaactgtcactcattgctgagcctctgctccccaggggagttgtgtctgtaatcgccctactattcagtggcgagaaataaagtttgcttagaaaagaaa

SEQ ID NO: 13 - protein, 837 aa, Homo sapiens
MSQTGSHPGRGLAGRWLWGAQPCLLLPIVPLSWLVWLLLLLLASLLPSARLASPLPREEEIVFPEKLNGSVLPGSGAPARLLCRLQAFGETLLLELEQDSGVQVEGLTVQYLGQAPELLGGAEPGTYLTGTINGDPESVASLHWDGGALLGVLQYRGAELHLQPLEGGTPNSAGGPGAHILRRKSPASGQGPMCNVKAPLGSPSPRPRRAKRFASLSRFVETLVVADDKMAAFHGAGLKRYLLTVMAAAAKAFKHPSIRNPVSLVVTRLVILGSGEEGPQVGPSAAQTLRSFCAWQRGLNTPEDSDPDHFDTAILFTRQDLCGVSTCDTLGMADVGTVCDPARSCAIVEDDGLQSAFTAAHELGHVFNMLHDNSKPCISLNGPLSTSRHVMAPVMAHVDPEEPWSPCSARFITDFLDNGYGHCLLDKPEAPLHLPVTFPGKDYDADRQCQLTFGPDSRHCPQLPPPCAALWCSGHLNGHAMCQTKHSPWADGTPCGPAQACMGGRCLHMDQLQDFNIPQAGGWGPWGPWGDCSRTCGGGVQFSSRDCTRPVPRNGGKYCEGRRTRFRSCNTEDCPTGSALTFREEQCAAYNHRTDLFKSFPGPMDWVPRYTGVAPQDQCKLTCQAQALGYYYVLEPRVVDGTPCSPDSSSVCVQGRCIHAGCDRIIGSKKKFDKCMVCGGDGSGCSKQSGSFRKFRYGYNNVVTIPAGATHILVRQQGNPGHRSIYLALKLPDGSYALNGEYTLMPSPTDVVLPGAVSLRYSGATAASETLSGHGPLAQPLTLQVLVAGNPQDTRLRYSFFVPRPTPSTPRPTPQDWLHRRAQILEILRRRPWAGRK

SEQ ID NO: 14 - DNA, 9777 bp, Homo sapiens
  source 1..9777: mol_type = other DNA
gggagaacccacagggagacccacagacacatatgcacgagagagacagaggaggaaagagacagagacaaaggcacagcggaagaaggcagagacagggcaggcacagaagcggcccagacagagtcctacagagggagaggccagagaagctgcagaagacacaggcagggagagacaaagatccaggaaaggagggctcaggaggagagtttggagaagccagacccctgggcacctctcccaagcccaaggactaagttttctccatttcctttaacggtcctcagcccttctgaaaactttgcctctgaccttggcaggagtccaagcccccaggctacagagaggagctttccaaagctagggtgtggaggacttggtgccctagacggcctcagtccctcccagctgcagtaccagtgccatgtcccagacaggctcgcatcccgggaggggcttggcagggcgctggctgtggggagcccaaccctgcctcctgctccccattgtgccgctctcctggctggtgtggctgcttctgctactgctggcctctctcctgccctcagcccggctggccagccccctcccccgggaggaggagatcgtgtttccagagaagctcaacggcagcgtcctgcctggctcgggcgcccctgccaggctgttgtgccgcttgcaggcctttggggagacgctgctactagagctggagcaggactccggtgtgcaggtcgaggggctgacagtgcagtacctgggccaggcgcctgagctgctgggtggagcagagcctggcacctacctgactggcaccatcaatggagatccggagtcggtggcatctctgcactgggatgggggagccctgttaggcgtgttacaatatcggggggctgaactccacctccagcccctggagggaggcacccctaactctgctgggggacctggggctcacatcctacgccggaagagtcctgccagcggtcaaggtcccatgtgcaacgtcaaggctcctcttggaagccccagccccagaccccgaagagccaagcgctttgcttcactgagtagatttgtggagacactggtggtggcagatgacaagatggccgcattccacggtgcggggctaaagcgctacctgctaacagtgatggcagcagcagccaaggccttcaagcacccaagcatccgcaatcctgtcagcttggtggtgactcggctagtgatcctggggtcaggcgaggaggggccccaagtggggcccagtgctgcccagaccctgcgcagcttctgtgcctggcagcggggcctcaacacccctgaggactcggaccctgaccactttgacacagccattctgtttacccgtcaggacctgtgtggagtctccacttgcgacacgctgggtatggctgatgtgggcaccgtctgtgacccggctcggagctgtgccattgtggaggatgatgggctccagtcagccttcactgctgctcatgaactgggtcatgtcttcaacatgctccatgacaactccaagccatgcatcagtttgaatgggcctttgagcacctctcgccatgtcatggcccctgtgatggctcatgtggatcctgaggagccctggtccccctgcagtgcccgcttcatcactgacttcctggacaatggctatgggcactgtctcttagacaaaccagaggctccattgcatctgcctgtgactttccctggcaaggactatgatgctgaccgccagtgccagctgaccttcgggcccgactcacgccattgtccacagctgccgccgccctgtgctgccctctggtgctctggccacctcaatggccatgccatgtgccagaccaaacactcgccctgggccgatggcacaccctgcgggcccgcacaggcctgcatgggtggtcgctgcctccacatggaccagctccaggacttcaatattccacaggctggtggctggggtccttggggaccatggggtgactgctctcggacctgtgggggtggtgtccagttctcctcccgagactgcacgaggcctgtcccccggaatggtggcaagtactgtgagggccgccgtacccgcttccgctcctgcaacactgaggactgcccaactggctcagccctgaccttccgcgaggagcagtgtgctgcctacaaccaccgcaccgacctcttcaagagcttcccagggcccatggactgggttcctcgctacacaggcgtggccccccaggaccagtgcaaactcacctgccaggcccaggcactgggctactactatgtgctggagccacgggtggtagatgggaccccctgttccccggacagctcctcggtctgtgtccagggccgatgcatccatgctggctgtgatcgcatcattggctccaagaagaagtttgacaagtgcatggtgtgcggaggggacggttctggttgcagcaagcagtcaggctccttcaggaaattcaggtacggatacaacaatgtggtcactatccccgcgggggccacccacattcttgtccggcagcagggaaaccctggccaccggagcatctacttggccctgaagctgccagatggctcctatgccctcaatggtgaatacacgctgatgccctcccccacagatgtggtactgcctggggcagtcagcttgcgctacagcggggccactgcagcctcagagacactgtcaggccatgggccactggcccagcctttgacactgcaagtcctagtggctggcaacccccaggacacacgcctccgatacagcttcttcgtgccccggccgaccccttcaacgccacgccccactccccaggactggctgcaccgaagagcacagattctggagatccttcggcggcgcccctgggcgggcaggaaataacctcactatcccggctgccctttctgggcaccggggcctcggacttagctgggagaaagagagagcttctgttgctgcctcatgctaagactcagtggggaggggctgtgggcgtgagacctgcccctcctctctgccctaatgcgcaggctggccctgccctggtttcctgccctgggaggcagtgatgggttagtggatggaaggggctgacagacagccctccatctaaactgccccctctgccctgcgggtcacaggagggagggggaaggcagggagggcctgggccccagttgtatttatttagtatttattcacttttatttagcaccagggaaggggacaaggactagggtcctggggaacctgacccctgacccctcatagccctcaccctggggctaggaaatccagggtggtggtgataggtataagtggtgtgtgtatgcgtgtgtgtgtgtgtgaaaatgtgtgtgtgcttatgtatgaggtacaacctgttctgctttcctcttcctgaattttattttttgggaaaagaaaagtcaagggtagggtgggccttcagggagtgagggattatctttttttttttttctttctttctttcttttttttttttgagacagaatctcgctctgtcgcccaggctggagtgcaatggcacaatctcggctcactgcatcctccgcctcccgggttcaagtgattctcatgcctcagcctcctgagtagctgggattacaggctcctgccaccacgcccggctaatttttgttttgttttgtttggagacagagtctcgctattgtcaccagggctggaatgatttcagctcactgcaaccttcgccacctgggttccagcaattctcctgcctcagcctcccgagtagctgagattataggcacctaccaccacgcccggctaatttttgtatttttagtagagacggggtttcaccatgttggccaggctggtctcgaactcctgaccttaggtgatccactcgccttcatctcccaaagtgctgggattacaggcgtgagccaccgtgcctggccacgcccaactaatttttgtatttttagtagagacagggtttcaccatgttggccaggctgctcttgaactcctgacctcaggtaatcgacctgcctcggcctcccaaagtgctgggattacaggtgtgagccaccacgcccggtacatattttttaaattgaattctactatttatgtgatccttttggagtcagacagatgtggttgcatcctaactccatgtctctgagcattagatttctcatttgccaataataatacctcccttagaagtttgttgtgaggattaaataatgtaaataaagaactagcataacactcagcatctagtaagtgctcaacaaatagcagctgctgttacttactgttatcaaatttctgtccacatccactctccatatgcacttgaaggtggcaaagatccacaaccatggtgcctgcctttatcctcagggtccgttcctttggttggcagacccctatcctgggttctgagggaccaacagagaaaggaaaattccatccctcacctctggaagttcccaatcacaggaaggaaacatagtaagcacgtggctacaaatacaattgacaagaacatgaaggtgcaggataacaagaacaaataacaagaacaactgcatcaacacaaatgagtgcttagtaataagggtgatagttgaggggtctgggtttcacaacagtagaaagagcactggagtgggagccagcgggtctggattcaatttggggctcggcgtcttattagctgggtggtgttgggtaagtcactgatgctgagccttagattgctcatatgggactaacagtatctactcccacagagttgttctgggaacaaatgctagaatattttcaaaatagtaaaggttatagtcatggccatgtgagaggttacccctatgactacctgaagatggaacggagtctccagaatctgccagtgtaaacccagcagaatgcctagaagatgtgagattagaataaaatttcataaaacaaaaacaatcgggcacggtggctcatgcctgtaatcccagcactttgggaggccgaaatgggcggatcacgaggtcaggagattgagaccgaccatcctggctaccacagtgaaaccccgtgtctactaaaaatacaaaatatatatatatatatatatatatattagccgggcatggtgacaggtgcctatagtcccagctacttgggagactgaggcaggagaatgacttgcacccgggaggcagagcttacagtgagtcgagattgcgccactgcactccagcctgggagacagagtgagactcgtctcgatacaaaaacaaaaacaaaaacaggatatggtttggcaggaaataggcaagaaggcaaaaagaataacctggaaaaggatctgaggtaagggagccaggtgcctcaaaatggcagaatacctgatgcctgaggagagggagggaataaaacatctgcattttcccctctgggcaaggcgcctttgcttgagaaagaattttgggctggatgagatggttcacacctgtaatcccagtactttggcaggaggattgcttgaggccaggaggttgggaccaacctgggcaacatagaaagattccatctctaccaaaaaaaaaaaagattgaaaaattagccgggcgtggtggcacctgtagtcccagctacttgggaggctaaaataggaggactgcttgagcccacgatttcgaggctatggtgagctacaatcatgccactatactccagcctgggtgagagaccaaaacaccaactcaaaaaaaaaaaaaaaaaaaaaaaaagggccaggcatggtggctcacacctgtaatctcagcactttgggaggctgaggttcaaaaccagccaggccaacatggtgaaaccccatctctactaaaaaaaaaatacaaaaaattagccaggtgtggtggcaagcgcctatagtcccagctactcatgaggctgaggcaggaggatcgcttgaacctgggaggtggaggttgcagtgagccgaaattgcgccattgcactccagtctgggcgatagagtgagactccatctcaaaaaaaaaaaaaaaaaaaaaaagaacttgggcagtcctctatgtgtcatggatggaacagggatgggcaagggtggtaggtagaccctgcaagaatttggagttttgagaggcagacgcaggactgctagggattggggaaggtactggatgtggggttgtgggagaaactataggtaaagaagaccctgaggttgaggtggaagagtgaagaatgggggaaccaaaggcacttcactctgccatagcagcccctagctgggatgccaaatactgcttggaatgtgaagctgggactatggggttgaggcaggccatggaggttgtagtggttatatgtgctatgcttactggatctgggctttgctaatcaagttctgtaccaggcagtgccttatacagcaccctgtactctacaccagccagcacagcgcctgcttcctctgcagcaatgagaaaagactgcccacactctcttctctccagtaaacacggttctccctgctaggcttggccccctggcccttcctgtggtcctcctactaaccaggctgaagaagatggagacaagacaatagtgatctttactcgtttcatctagtttgcaaaatgagaccacagatagtatgtttatggaccttgatactatgagttgatggtacaatgagcagagttctgtgataagtagatgtaaggtacaaatacaaacaatatatacgactgtcctaaaggggttcacactgcattctgggcaggggaggttgtatactgctcccactccaccttccccgcaaacagaagcaatgggacagtaaaagggactagaagataagagcctaatgagccctgaactgaatggactatgagggttatggagggctaccttgggctgggccctggagaacaggtcatcatgatagcccttctcactttcctttaatgttcatggagatacacttttttttttgagatagggtctccctttgttgaccaggcaggagtacagtggtgtgatcttggctcactgcagccttgaactctcaggctcaagcaaccctcccacctcaacctcctgagtacctaggactacaggcacgagctaccacacccagctaatttacttttttttttggttgagacgaggtcttatcatattgcccaggctggtcttgaactcctggcctcaagggatcttcccacctcgatctcccaaagtgttgggatgaccagtgtgaaccaccatgcctggccttttcttttgagacagtctcgctctgtggctcaggttggagtgcagtggcaccatcataacttactacagccttgaactcctgggctcgagtaatcctcccacctcagcctcctaagtagcttggactacaggtgtagattcacttttaaatcacttttctgcctgttattttaagagaaagcccttcgctcaccaagtagggttctcactggcataagtaagtttttctgaaaatgagactgagcttaggggccacctctgtgattgggtaagtaagcaggagctagagtgaaagaggggcagagagagctagctcatcgaagagaagtgatgggcatctcagccctcaatcccaggcaaggcctgacccttttctggaatggtctctgccttaccacacctccaagacccagattgtaggtaggctttacctaataggagtccaacaagttcttgctagggatgggattgtgggggggcacatatctccctgtaagagccacagtgagctctgtttcccatctgcctacttattgccctcgtgggcctgagctggggtgtagatatataaggaggaacagaaacagtaataacaggaagtaaggaggcactgaaacaattcaagtatctggtcctggagatgaaaagaggaaatggaaaaagaaaaggcattgaatgacaagggattagaaccctgtccctagagaagttcaagaagagtttggacaaccattttatgagatggcaagtgttgtgcatgagtatgtgtttgaggtgggggaggaagttgaagcaataagttgggccaatttcctaaagtcccttctgcccctgagattctctgcttctgtggttcaaacagaggaaatctggacatttgtccagccactatttttgtaatggaggtgggacagccagggttcctggagtgtgctgtggaatcggcatttacacttctcttccatctctagtctcatttctgtaatcttcatacagtattacagtattttgttttctaaaataccagattggcagtaagtgacaaaactagcatctcattggtcagggcttgtgggggaggaagggagaagaaagtggatttcaatgtattaacattttattggctgagaggatgtttctgaaccaattgagtaccttccccattgactgttgctggggccaagccaaaatcattggcaccacgagaaaccagatgacagctggaggaaaccacgagtgctaaaaatgctcagaaagaggggggtcagggaaggcgggggagcaacaacactgaacaacttccccaggcaggatcttacatggggacagacccagggtacttaagtagcattaggaaggaaagggaggggaggaagtagattaagtatccctcacagtcttggcacaacaaacaggcaccatacacccacaccaacagtgacacattgacataacacacatccaacttcacagagacacactagcacattctctcttttttttaaaatttattttattattattatactttaagttttagggtacatgtgcacaatgtgcaggttagttacatatgtatacatgtgccatgctggtgtgctgcacccattaactcatcatttagcattaggtatatctcctaatgctatccctcccccctccccccctgcctttctttttcttcttttttttcttaagacagattctcattctgtcacccaggctagagtgcaatggcgtgatctcggctcactgcaacctccacctcccaggttcaagtgattctcctgcctcagcctgccaagtagctgggattacaggtatgcaccaccatgcccagctaattttttgtatttttagtatagagacggggttttccatgttgcccaggctggtcttgaactcccgggctcaagtgatctgcctgcctcggcctcccaaagtgctgggattacaggtgtgagccagggtgcctggccaacacattctgttgataatatagatgcaaataagtagtagggagcggaaaatgtaagaattttcacctgaaaatggatctgaagagtaaaagatattaattgacctaatcgtgcattgagaattgactgtttatcccatttatttgttcaataaataagccaatgggttgtaaacattgtaaacattccacagcatgtggaaaggacatggatttttggagtcaaattgaatctaagccttgctacttacttacctgtaatgatgcaaccttaattacttgatctctgtaaacctcagtttccttacctacaaaacgtttgtactattacctgccttatagagaggtccagacttaagaatatgatgatgtaaatgtttggcactgtgtctggcttttagtaagtggtcaataaaggccagttctcctttcctta

SEQ ID NO: 15 - protein, 830 aa, Homo sapiens
MANCQIAILYQRFQRVVFGISQLLCFSALISELTNQKEVAAWTYHYSTKAYSWNISRKYCQNRYTDLVAIQNKNEIDYLNKVLPYYSSYYWIGIRKNNKTWTWVGTKKALTNEAENWADNEPNNKRNNEDCVEIYIKSPSAPGKWNDEHCLKKKHALCYTASCQDMSCSKQGECLETIGNYTCSCYPGFYGPECEYVRECGELELPQHVLMNCSHPLGNFSFNSQCSFHCTDGYQVNGPSKLECLASGIWTNKPPQCLAAQCPPLKIPERGNMTCLHSAKAFQHQSSCSFSCEEGFALVGPEVVQCTASGVWTAPAPVCKAVQCQHLEAPSEGTMDCVHPLTAFAYGSSCKFECQPGYRVRGLDMLRCIDSGHWSAPLPTCEAISCEPLESPVHGSMDCSPSLRAFQYDTNCSFRCAEGFMLRGADIVRCDNLGQWTAPAPVCQALQCQDLPVPNEARVNCSHPFGAFRYQSVCSFTCNEGLLLVGASVLQCLATGNWNSVPPECQAIPCTPLLSPQNGTMTCVQPLGSSSYKSTCQFICDEGYSLSGPERLDCTRSGRWTDSPPMCEAIKCPELFAPEQGSLDCSDTRGEFNVGSTCHFSCDNGFKLEGPNNVECTTSGRWSATPPTCKGIASLPTPGLQCPALTTPGQGTMYCRHHPGTFGFNTTCYFGCNAGFTLIGDSTLSCRPSGQWTAVTPACRAVKCSELHVNKPIAMNCSNLWGNFSYGSICSFHCLEGQLLNGSAQTACQENGHWSTTVPTCQAGPLTIQEALTYFGGAVASTIGLIMGGTLLALLRKRFRQKDDGKCPLNPHSHLGTYGVFTNAAFDPSP

SEQ ID NO: 16 - DNA, 3157 bp, Homo sapiens
  source 1..3157: mol_type = other DNA
agcagtctgggttgggcagaaggcagaaaaccagcagagtcacagaggagatggccaactgccaaatagccatcttgtaccagagattccagagagtggtctttggaatttcccaactcctttgcttcagtgccctgatctctgaactaacaaaccagaaagaagtggcagcatggacttatcattacagcacaaaagcatactcatggaatatttcccgtaaatactgccagaatcgctacacagacttagtggccatccagaataaaaatgaaattgattacctcaataaggtcctaccctactacagctcctactactggattgggatccgaaagaacaataagacatggacatgggtgggaaccaaaaaggctctcaccaacgaggctgagaactgggctgataatgaacctaacaacaaaaggaacaacgaggactgcgtggagatatacatcaagagtccgtcagcccctggcaagtggaatgatgagcactgcttgaagaaaaagcacgcattgtgttacacagcctcctgccaggacatgtcctgcagcaaacaaggagagtgcctcgagaccatcgggaactacacctgctcctgttaccctggattctatgggccagaatgtgaatacgtgagagagtgtggagaacttgagctccctcaacacgtgctcatgaactgcagccaccctctgggaaacttctcttttaactcgcagtgcagcttccactgcactgacgggtaccaagtaaatgggcccagcaagctggaatgcttggcttctggaatctggacaaataagcctccacagtgtttagctgcccagtgcccacccctgaagattcctgaacgaggaaacatgacctgccttcattctgcaaaagcattccagcatcagtctagctgcagcttcagttgtgaagagggatttgcattagttggaccggaagtggtgcaatgcacagcctcgggggtatggacagccccagccccagtgtgtaaagctgtgcagtgtcagcacctggaagcccccagtgaaggaaccatggactgtgttcatccgctcactgcttttgcctatggctccagctgtaaatttgagtgccagcccggctacagagtgaggggcttggacatgctccgctgcattgactctggacactggtctgcacccttgccaacctgtgaggctatttcgtgtgagccgctggagagtcctgtccacggaagcatggattgctctccatccttgagagcgtttcagtatgacaccaactgtagcttccgctgtgctgaaggtttcatgctgagaggagccgatatagttcggtgtgataacttgggacagtggacagcaccagccccagtctgtcaagctttgcagtgccaggatctcccagttccaaatgaggcccgggtgaactgctcccaccccttcggtgcctttaggtaccagtcagtctgcagcttcacctgcaatgaaggcttgctcctggtgggagcaagtgtgctacagtgcttggctactggaaactggaattctgttcctccagaatgccaagccattccctgcacacctttgctaagccctcagaatggaacaatgacctgtgttcaacctcttggaagttccagttataaatccacatgtcaattcatctgtgacgagggatattctttgtctggaccagaaagattggattgtactcgatcgggacgctggacagactccccaccaatgtgtgaagccatcaagtgcccagaactctttgccccagagcagggcagcctggattgttctgacactcgtggagaattcaatgttggctccacctgccatttctcttgtgacaacggctttaagctggaggggcccaataatgtggaatgcacaacttctggaagatggtcagctactccaccaacctgcaaaggcatagcatcacttcctactccaggggtgcaatgtccagccctcaccactcctgggcagggaaccatgtactgtaggcatcatccgggaacctttggttttaataccacttgttactttggctgcaacgctggattcacactcataggagacagcactctcagctgcagaccttcaggacaatggacagcagtaactccagcatgcagagctgtgaaatgctcagaactacatgttaataagccaatagcgatgaactgctccaacctctggggaaacttcagttatggatcaatctgctctttccattgtctagagggccagttacttaatggctctgcacaaacagcatgccaagagaatggccactggtcaactaccgtgccaacctgccaagcaggaccattgactatccaggaagccctgacttactttggtggagcggtggcttctacgataggtctgataatgggtgggacgctcctggctttgctaagaaagcgtttcagacaaaaagatgatgggaaatgccccttgaatcctcacagccacctaggaacatatggagtttttacaaacgctgcatttgacccgagtccttaaggtttccataaacacccatgaatcaaagacatggaattaccttagattagctctggaccagcctgttggacccgctctggaccaaccctgtttcctgagtttgggattgtggtacaatctcaaattctcaacctaccaccccttcctgtcccacctcttctcttcctgtaacacaagccacagaagccaggagcaaatgtttctgcagtagtctctgtgctttgactcacctgttacttgaaataccagtgaaccaaagagactggagcatctgactcacaagaagaccagactgtggagaaataaaaatacctctttattttttgattgaaggaaggttttctccactttgttggaaagcaggtggcatctctaattggaagaaattcctgtagcatcttctggagtctccagtggttgctgttgatgaggcctcttggacctctgctctgaggcttccagagagtcctctggatggcaccagaggctgcagaaggccaagaatcaagctagaaggccacatgtcaccgtggaccttcctgccaccagtcactgtccctcaaatgacccaaagaccaatattcaaatgcgtaattaaaagaattttccccaaa

SEQ ID NO: 17 - protein, 626 aa, Homo sapiens
MPCVQAQYSPSPPGSSYAAQTYSSEYTTEIMNPDYTKLTMDLGSTEITATATTSLPSISTFVEGYSSNYELKPSCVYQMQRPLIKVEEGRAPSYHHHHHHHHHHHHHHQQQHQQPSIPPASSPEDEVLPSTSMYFKQSPPSTPTTPAFPPQAGALWDEALPSAPGCIAPGPLLDPPMKAVPTVAGARFPLFHFKPSPPHPPAPSPAGGHHLGYDPTAAAALSLPLGAAAAAGSQAAALESHPYGLPLAKRAAPLAFPPLGLTPSPTASSLLGESPSLPSPPSRSSSSGEGTCAVCGDNAACQHYGVRTCEGCKGFFKRTVQKNAKYVCLANKNCPVDKRRRNRCQYCRFQKCLSVGMVKEVVRTDSLKGRRGRLPSKPKSPLQQEPSQPSPPSPPICMMNALVRALTDSTPRDLDYSRYCPTDQAAAGTDAEHVQQFYNLLTASIDVSRSWAEKIPGFTDLPKEDQTLLIESAFLELFVLRLSIRSNTAEDKFVFCNGLVLHRLQCLRGFGEWLDSIKDFSLNLQSLNLDIQALACLSALSMITERHGLKEPKRVEELCNKITSSLKDHQSKGQALEPTESKVLGALVELRKICTLGLQRIFYLKLEDLVSPPSIIDKLFLDTLPF

SEQ ID NO: 18 - DNA, 5604 bp, Homo sapiens
  source 1..5604: mol_type = other DNA
gcgcagccgggagagcggagtctcctgcctcccgccccccacccctccagctcctgctcctcctccgctccccatacacagacgcgctcacacccgctccctcactcgcacacacagacacaagcgcgcacacaggctccgcacacacacttcgctctcccgcgcgctcacacccctcttgccctgagcccttgccggtgcagcgcggcgccgcagctggacgcccctcccgggctcactttgcaacgctgacggtgccggcagtggccgtggaggtgggaacagcggcggcatcctcccccctggtcacagcccaagccaggacgcccgcggaacctctcggctgtgctctcccatgagtcgggatcgcagcatcccccaccagccgctcaccgcctccgggagccgctgggcttgtacaccgcagcccttccgggacagcagctgtgactcccccccagtgcagatttcgggacagctctctagaaactcgctctaaagacggaaccgccacagcactcaaagcccactgcggaagagggcagcccggcaagcccgggccctgagcctggacccttagcggtgccgggcagcactgccggcgcttcgcctcgccggacgtccgctcctcctacactctcagcctccgctggagagacccccagccccaccattcagcgcgcaagataccctccagatatgccctgcgtccaagcccaatatagcccttcccctccaggttccagttatgcggcgcagacatacagctcggaatacaccacggagatcatgaaccccgactacaccaagctgaccatggaccttggcagcactgagatcacggctacagccaccacgtccctgcccagcatcagtaccttcgtggagggctactcgagcaactacgaactcaagccttcctgcgtgtaccaaatgcagcggcccttgatcaaagtggaggaggggcgggcgcccagctaccatcaccatcaccaccaccaccaccaccaccaccaccatcaccagcagcagcatcagcagccatccattcctccagcctccagcccggaggacgaggtgctgcccagcacctccatgtacttcaagcagtccccaccgtccacccccaccacgccggccttccccccgcaggcgggggcgttatgggacgaggcactgccctcggcgcccggctgcatcgcacccggcccgctgctggacccgccgatgaaggcggtccccacggtggccggcgcgcgcttcccgctcttccacttcaagccctcgccgccgcatccccccgcgcccagcccggccggcggccaccacctcggctacgacccgacggccgctgccgcgctcagcctgccgctgggagccgcagccgccgcgggcagccaggccgccgcgcttgagagccacccgtacgggctgccgctggccaagagggcggccccgctggccttcccgcctctcggcctcacgccctcccctaccgcgtccagcctgctgggcgagagtcccagcctgccgtcgccgcccagcaggagctcgtcgtctggcgagggcacgtgtgccgtgtgcggggacaacgccgcctgccagcactacggcgtgcgaacctgcgagggctgcaagggctttttcaagagaacagtgcagaaaaatgcaaaatatgtttgcctggcaaataaaaactgcccagtagacaagagacgtcgaaaccgatgtcagtactgtcgatttcagaagtgtctcagtgttggaatggtaaaagaagttgtccgtacagatagtctgaaagggaggagaggtcgtctgccttccaaaccaaagagcccattacaacaggaaccttctcagccctctccaccttctcctccaatctgcatgatgaatgcccttgtccgagctttaacagactcaacacccagagatcttgattattccagatactgtcccactgaccaggctgctgcaggcacagatgctgagcatgtgcaacaattctacaacctcctgacagcctccattgatgtatccagaagctgggcagaaaagattccgggatttactgatctccccaaagaagatcagacattacttattgaatcagcctttttggagctgtttgtcctcagactttccatcaggtcaaacactgctgaagataagtttgtgttctgcaatggacttgtcctgcatcgacttcagtgccttcgtggatttggggagtggctcgactctattaaagacttttccttaaatttgcagagcctgaaccttgatatccaagccttagcctgcctgtcagcactgagcatgatcacagaaagacatgggttaaaagaaccaaagagagtcgaagagctatgcaacaagatcacaagcagtttaaaagaccaccagagtaagggacaggctctggagcccaccgagtccaaggtcctgggtgccctggtagaactgaggaagatctgcaccctgggcctccagcgcatcttctacctgaagctggaagacttggtgtctccaccttccatcattgacaagctcttcctggacaccctacctttctaatcaggagcagtggagcagtgagctgcctcctctcctagcacctgcttgctacgcagcaaagggataggtttggaaacctatcatttcctgtccttccttaagaggaaaagcagctcctgtagaaagcaaagactttcttttttttctggctcttttccttacaacctaaagccagaaaacttgcagagtattgtgttggggttgtgttttatatttaggcattgggggatggggtgggagggggttatagttcatgagggttttctaagaaattgctaacaaagcacttttggacaatgctatcccagcaggaaaaaaaaggataatataactgttttaaaactctttctggggaatccaattatagttgctttgtatttaaaaacaagaacagccaagggttgttcgccagggtaggatgtgtcttaaagattggtcccttgaaaatatgcttcctgtatcaaaggtacgtatgtggtgcaaacaaggcagaaacttccttttaatttccttcttcctttattttaacaaatggtgaaagatggaggattacctacaaatcagacatggcaaaacaataatggctgtttgcttccataaacaagtgcaattttttaaagtgctgtcttactaagtcttgtttattaactctcctttattctatatggaaataaaaaggaggcagtcatgttagcaaatgacacgttaatatccctagcagaggctgtgttcaccttccctgtcgatcccttctgaggtatggcccatccaagacttttaggccattcttgatggaaccagatccctgccctgactgtccagctatcctgaaagtggatcagattataaactggattacatgtaactgttttggttgtgttctatcaaccccaccagagttccctaaacttgcttcagttatagtaactgactggtatattcattcagaagcgccataagtcagttgagtatttgatccctagataagaacatgcaaatcagcaggaactggtcatacagggtaagcaccagggacaataaggatttttatagatataatttaatttttgttattggttaaggagacaattttggagagcaagcaaatctttttaaaaaatagtatgaatgtgaatactagaaaagatttaaaaaatagtatgagtgtgagtactaggaaggattagtgggctgcgtttcaacattccgtgttcgtactcccttttgtatgtttctactgttaatgccatattactatgagataatttgttgcatagtgtccttatttgtataaacatttgtatgcacgttatattgtaatagctttgcctgtatttattgcaagaccaccagctcctggaagctgagttacagagtaattaaatggggtgttcacagtgacttggatacaccaattagaaattaaataagcaaatatatatatatatataaatatagcaggttacatatatatatttataatgtgtctttttattaaccatttgtacaataaatgtcacttcccatgccgttattttatggttcatttgcagtgacttttaaggcagtactgtttagcactttgatattaaaattttgcttatgttttgctaaattcgaataatgtttgaagatttttaggtctaaaagtctttatattatatactctgtatcaagtcaaaatatctttggccattttgctaagaaacaaactttgaatgtcaaactgatgtcacagtagtttttgttagctttaaatcatttttgctttagtctttttaaaggaaaataacaaaactatgctgtttatattgtcattaaattatacaatcaaacaaatgccaaatgaattgcctaattgctgcaaagtataacccagataggaaatcatatgtttttttccaagagtcattctaatatttgattatgttatgtgtgcttttatgaaagattgttatttttatatatcaagatgatagaacctggaatgttaggattttgaaatgttagacttggaaggggcctggtctgtcaactagtccaaccccttaaaattcatagaggagcaaactggggcccattgaagggtgaagagttactcaaggtcaaacagctggtaacagaatcaagactaagacctaatttacctttccatactctttttttttctcaacttcatctatataaaatcaggcttttaaacataaccactaatatttacctgaagataaccatgagtaaagtatacttttgcattaattttttgagcttatatgcaaacataataaatattattaaatatcaggaaagctaacatttcatacaagatagcttcagaccaaattcaaattgaatttgaataaattagaaatactgtgcatacataaccttcttgtgcaccatgagtatttggaaagttaatccttgtttttgtcgtgtctataaaggaagaacaaaacaaaataaaaacagagccctagagaaatgctgttactttttatttttacacccatcagatttaaggaaaagactttttagccattataatctagtggttggaaggaatgaagaagcttttttagtaataggtccagatatgagtgctaaaaataaagatgatagcatgttcttctgtcttccatagttattacaactatgagagcctcccaagtcatcttatcaactcaactcccttttttttgtcttaatgttgcacataagtttatacagagtggatgaccacactagcacagaagagaacaacatgtattaaagcaggtgattcctccccttggcgggagagctctctcagtgtgaacatgccttctgtgggcggaaatcaggaagccaccagctgttaatggagagtgccttgcttttatttcagacagcagagttttccaaagtttctctgctcctctaacagcattgctctttagtgtgtgttaacctgtggtttgaaagaaatgctcttgtacattaacaatgtaaatttaaatgattaaattacattttatcaatggc